SEQUENCE LISTING (78 sequences)

SEQ ID NO: 1 - protein, 114 aa, Artificial Sequence
  Anti-CD47 VH
QVQLVQSGAEVKKPGASVKVSCKASGYTFTSYWMHWVRQAPGQGLEWIGNIDPSDSETHYAQKFQGRVTLTVDKSTSTVYMELSSLRSEDTAVYYCAGTDLAYWGQGTLVTVSS

SEQ ID NO: 2 - protein, 107 aa, Artificial Sequence
  Anti-CD47 VL
EIVLTQSPGTLSLSPGERATLSCHASQNINVWLSWYQQKPGQAPRLLIYKASNLHTGIPDRFSGSGSGTDFTLTISRLEPEDFAVYYCQQGQSYPFTFGQGTKVEIK

SEQ ID NO: 3 - protein, 119 aa, Artificial Sequence
  Anti-CLDN18.2 VH
EVQLVESGGGLVQPGGSLRLSCAASGFIFSSFGMHWVRQAPGKGLEWVAYISSGRSTMYYADSVKGRFTISRDNSKNTLYLQMNSLTAEDTAVYYCARGGFYGNSLDYWGQGTLVTVSS

SEQ ID NO: 4 - protein, 113 aa, Artificial Sequence
  Anti-CLDN18.2 VL
DIQMTQSPSSLSASVGDRVTITCKSSLSLLNSGNQKNYLTWYQQKPGKAPKLLIYWASTRESGVPSRFSGSGSGTDFTLTISSLQPEDFATYSCQNAYSYPLTFGQGTKVEIK

SEQ ID NO: 5 - protein, 119 aa, Artificial Sequence
  Anti-CD3 VH
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYSLDYWGQGTTLTVSS

SEQ ID NO: 6 - protein, 106 aa, Artificial Sequence
  Anti-CD3 VL
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 7 - protein, 118 aa, Artificial Sequence
  Anti-DLL3 VH
EVRLSQSGGQMKKPGESMRLSCRASGYTFTSYVMHWVRQAPGRRPEWIGYINPYNDATKYARKFQGRATLTSDKYSDTAFLELRSLTSDDTAVYYCARGGYDYDGDYWGRGAPVTVSS

SEQ ID NO: 8 - protein, 107 aa, Artificial Sequence; identical to SEQ ID NO: 2
  Anti-DLL3 VL

SEQ ID NO: 9 - protein, 119 aa, Artificial Sequence
  Anti-CD3 VH_33
DIKLQQSGAELARPGASVKMSCKTSGYTFTRYTMHWVKKRPGQGLEWIGYINPSRGYTNYNQKFKDKATLTTDKSSSTAYMQLSSLTSEDSAVYYCARYYDDHYSLDYWGQGTTLTVSS

SEQ ID NO: 10 - protein, 103 aa, Artificial Sequence
  Anti-CD3 CH1_33
ASTKGPSVFPLAPSSKSTSGGTAALGCLVKDYFPEPVTVSWNSGALTSGVHTFPAVLQSSGLYSLSSVVKVPSSSLGTQTYICNVNHKPSNTKVDKKVEPKSC

SEQ ID NO: 11 - protein, 106 aa, Artificial Sequence
  Anti-CD3 VL_33
DIQLTQSPAIMSASPGEKVTMTCRASSSVSYMNWYQEKSGTSPKRWIYDTSKVASGVPYRFSGSGSGTSYSLTISSMEAEDAATYYCQQWSSNPLTFGAGTKLELK

SEQ ID NO: 12 - protein, 107 aa, Artificial Sequence
  Anti-CD3 CL_33
RTVAAPSVFIFPPSDEQLKSGTASVVCLLNNFYPREAKVQWKVDNALQSGNSQESVTEQDSKESTYSLSSTLTLSKADYEKHKVYACEVTHQGLSSPVTKSFNRGEC

SEQ ID NO: 13 - protein, 118 aa, Artificial Sequence
  Anti-DLL3 VH_33
EVRLSQSGGQMKKPGESMRLSCRASGYTFTSYVMHWVREAPGRRPEWIGYINPYNDATKYARKFQGRATLTSDKYSDTAFLELRSLTSDDTAVYYCARGGYDYDGDYWGRGAPVTVSS

SEQ ID NO: 14 - protein, 103 aa, Artificial Sequence
  Anti-DLL3 CH1_33
ASTKGPSVFPLAPSSCSTSGGTAALGCLVKDYFPEPVTVSWNSGALTSGVHTFPAVLQSSGLYSLSSVVEVPSSSLGTQTYICNVNHKPSNTKVDKKVEPKSS

SEQ ID NO: 15 - protein, 107 aa, Artificial Sequence
  Anti-DLL3 VL_33
EIVLTQSPGTLSLSPGERATLSCHASQNINVWLSWYQKKPGQAPRLLIYKASNLHTGIPDRFSGSGSGTDFTLTISRLEPEDFAVYYCQQGQSYPFTFGQGTKVEIK

SEQ ID NO: 16 - protein, 107 aa, Artificial Sequence
  Anti-DLL3 CL_33
RTVAAPSVFIFPPSDEQLKSGTASVVCLLNNFYPREAKVQWKVDNALQSGNSQESVTEQDSKKSTYSLSSTLTLSKADYEKHKVYACEVTHQGLSSPVTKSCNRGES

SEQ ID NO: 17 - protein, 103 aa, Artificial Sequence
  IgG1 CH1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 18 - protein, 103 aa, Artificial Sequence
  IgG2 CH1
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCC

SEQ ID NO: 19 - protein, 105 aa, Artificial Sequence
  IgG3 CH1
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLG

SEQ ID NO: 20 - protein, 103 aa, Artificial Sequence
  IgG4 CH1
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYG

SEQ ID NO: 21 - protein, 107 aa, Artificial Sequence [sequence omitted]

SEQ ID NO: 22 - protein, 104 aa, Artificial Sequence
  Lambda CL
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTEC

SEQ ID NO: 23 - protein, 114 aa, Artificial Sequence
  Anti-CD47 VH Q39E
QVQLVQSGAEVKKPGASVKVSCKASGYTFTSYWMHWVREAPGQGLEWIGNIDPSDSETHYAQKFQGRVTLTVDKSTSTVYMELSSLRSEDTAVYYCAGTDLAYWGQGTLVTVSS

SEQ ID NO: 24 - protein, 107 aa, Artificial Sequence; identical to SEQ ID NO: 15
  Anti-CD47 VL Q38K

SEQ ID NO: 25 - protein, 114 aa, Artificial Sequence
  Anti-CD47 VH Q39K
QVQLVQSGAEVKKPGASVKVSCKASGYTFTSYWMHWVRKAPGQGLEWIGNIDPSDSETHYAQKFQGRVTLTVDKSTSTVYMELSSLRSEDTAVYYCAGTDLAYWGQGTLVTVSS

SEQ ID NO: 26 - protein, 107 aa, Artificial Sequence
  Anti-CD47 VL Q38E
EIVLTQSPGTLSLSPGERATLSCHASQNINVWLSWYQEKPGQAPRLLIYKASNLHTGIPDRFSGSGSGTDFTLTISRLEPEDFAVYYCQQGQSYPFTFGQGTKVEIK

SEQ ID NO: 27 - protein, 103 aa, Artificial Sequence
  Anti-CD47 CH1 G166D
ASTKGPSVFPLAPSSCSTSGGTAALGCLVKDYFPEPVTVSWNSGALTSDVHTFPAVLQSSGLYSLSSVVTVPSSSLGTQTYICNVNHKPSNTKVDKKVEPKSS

SEQ ID NO: 28 - protein, 103 aa, Artificial Sequence
  Anti-CD47 CH1 G166E
ASTKGPSVFPLAPSSCSTSGGTAALGCLVKDYFPEPVTVSWNSGALTSEVHTFPAVLQSSGLYSLSSVVTVPSSSLGTQTYICNVNHKPSNTKVDKKVEPKSS

SEQ ID NO: 29 - protein, 107 aa, Artificial Sequence
  Anti-CD47 CL S114K
RTVAAPKVFIFPPSDEQLKSGTASVVCLLNNFYPREAKVQWKVDNALQSGNSQESVTEQDSKDSTYSLSSTLTLSKADYEKHKVYACEVTHQGLSSPVTKSCNRGES

SEQ ID NO: 30 - protein, 107 aa, Artificial Sequence
  Anti-CD47 CL S114R
RTVAAPRVFIFPPSDEQLKSGTASVVCLLNNFYPREAKVQWKVDNALQSGNSQESVTEQDSKDSTYSLSSTLTLSKADYEKHKVYACEVTHQGLSSPVTKSCNRGES

SEQ ID NO: 31 - protein, 103 aa, Artificial Sequence
  Anti-CD47 CH1 G166K
ASTKGPSVFPLAPSSCSTSGGTAALGCLVKDYFPEPVTVSWNSGALTSKVHTFPAVLQSSGLYSLSSVVTVPSSSLGTQTYICNVNHKPSNTKVDKKVEPKSS

SEQ ID NO: 32 - protein, 103 aa, Artificial Sequence
  Anti-CD47 CH1 G166R
ASTKGPSVFPLAPSSCSTSGGTAALGCLVKDYFPEPVTVSWNSGALTSRVHTFPAVLQSSGLYSLSSVVTVPSSSLGTQTYICNVNHKPSNTKVDKKVEPKSS

SEQ ID NO: 33 - protein, 107 aa, Artificial Sequence
  Anti-CD47 CL S114D
RTVAAPDVFIFPPSDEQLKSGTASVVCLLNNFYPREAKVQWKVDNALQSGNSQESVTEQDSKDSTYSLSSTLTLSKADYEKHKVYACEVTHQGLSSPVTKSCNRGES

SEQ ID NO: 34 - protein, 107 aa, Artificial Sequence
  Anti-CD47 CL S114E
RTVAAPEVFIFPPSDEQLKSGTASVVCLLNNFYPREAKVQWKVDNALQSGNSQESVTEQDSKDSTYSLSSTLTLSKADYEKHKVYACEVTHQGLSSPVTKSCNRGES

SEQ ID NO: 35 - protein, 103 aa, Artificial Sequence
  Anti-CD47 CH1 T187D
ASTKGPSVFPLAPSSCSTSGGTAALGCLVKDYFPEPVTVSWNSGALTSGVHTFPAVLQSSGLYSLSSVVDVPSSSLGTQTYICNVNHKPSNTKVDKKVEPKSS

SEQ ID NO: 36 - protein, 103 aa, Artificial Sequence; identical to SEQ ID NO: 14
  Anti-CD47 CH1 T187E

SEQ ID NO: 37 - protein, 107 aa, Artificial Sequence; identical to SEQ ID NO: 16
  Anti-CD47 CL D170K

SEQ ID NO: 38 - protein, 107 aa, Artificial Sequence
  Anti-CD47 CL D170R
RTVAAPSVFIFPPSDEQLKSGTASVVCLLNNFYPREAKVQWKVDNALQSGNSQESVTEQDSKRSTYSLSSTLTLSKADYEKHKVYACEVTHQGLSSPVTKSCNRGES

SEQ ID NO: 39 - protein, 103 aa, Artificial Sequence
  Anti-CD47 CH1 T187K
ASTKGPSVFPLAPSSCSTSGGTAALGCLVKDYFPEPVTVSWNSGALTSGVHTFPAVLQSSGLYSLSSVVKVPSSSLGTQTYICNVNHKPSNTKVDKKVEPKSS

SEQ ID NO: 40 - protein, 103 aa, Artificial Sequence
  Anti-CD47 CH1 T187R
ASTKGPSVFPLAPSSCSTSGGTAALGCLVKDYFPEPVTVSWNSGALTSGVHTFPAVLQSSGLYSLSSVVRVPSSSLGTQTYICNVNHKPSNTKVDKKVEPKSS

SEQ ID NO: 41 - protein, 107 aa, Artificial Sequence
  Anti-CD47 CL D170D
RTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSCNRGES

SEQ ID NO: 42 - protein, 107 aa, Artificial Sequence
  Anti-CD47 CL D170E
RTVAAPSVFIFPPSDEQLKSGTASVVCLLNNFYPREAKVQWKVDNALQSGNSQESVTEQDSKESTYSLSSTLTLSKADYEKHKVYACEVTHQGLSSPVTKSCNRGES

SEQ ID NO: 43 - protein, 103 aa, Artificial Sequence
  Anti-CD47 CH1 S131D
ASTKGPSVFPLAPDSCSTSGGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 44 - protein, 103 aa, Artificial Sequence
  Anti-CD47 CH1 S131E
ASTKGPSVFPLAPESCSTSGGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 45 - protein, 107 aa, Artificial Sequence
  Anti-CD47 CL P119K
RTVAAPSVFIFKPSDEQLKSGTASVVCLLNNFYPREAKVQWKVDNALQSGNSQESVTEQDSKDSTYSLSSTLTLSKADYEKHKVYACEVTHQGLSSPVTKSCNRGES

SEQ ID NO: 46 - protein, 107 aa, Artificial Sequence
  Anti-CD47 CL P119R
RTVAAPSVFIFRPSDEQLKSGTASVVCLLNNFYPREAKVQWKVDNALQSGNSQESVTEQDSKDSTYSLSSTLTLSKADYEKHKVYACEVTHQGLSSPVTKSCNRGES

SEQ ID NO: 47 - protein, 103 aa, Artificial Sequence
  Anti-CD47 CH1 S131K
ASTKGPSVFPLAPKSCSTSGGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 48 - protein, 103 aa, Artificial Sequence
  Anti-CD47 CH1 S131R
ASTKGPSVFPLAPRSCSTSGGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 49 - protein, 107 aa, Artificial Sequence
  Anti-CD47 CL P119D
RTVAAPSVFIFDPSDEQLKSGTASVVCLLNNFYPREAKVQWKVDNALQSGNSQESVTEQDSKDSTYSLSSTLTLSKADYEKHKVYACEVTHQGLSSPVTKSCNRGES

SEQ ID NO: 50 - protein, 107 aa, Artificial Sequence
  Anti-CD47 CL P119E
RTVAAPSVFIFEPSDEQLKSGTASVVCLLNNFYPREAKVQWKVDNALQSGNSQESVTEQDSKDSTYSLSSTLTLSKADYEKHKVYACEVTHQGLSSPVTKSCNRGES

SEQ ID NO: 51 - protein, 103 aa, Artificial Sequence
  Anti-CD47 CH1 A129D
ASTKGPSVFPLDPSSCSTSGGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 52 - protein, 103 aa, Artificial Sequence
  Anti-CD47 CH1 A129E
ASTKGPSVFPLEPSSCSTSGGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 53 - protein, 107 aa, Artificial Sequence
  Anti-CD47 CL S121K
RTVAAPSVFIFPPKDEQLKSGTASVVCLLNNFYPREAKVQWKVDNALQSGNSQESVTEQDSKDSTYSLSSTLTLSKADYEKHKVYACEVTHQGLSSPVTKSCNRGES

SEQ ID NO: 54 - protein, 107 aa, Artificial Sequence
  Anti-CD47 CL S121R
RTVAAPSVFIFPPRDEQLKSGTASVVCLLNNFYPREAKVQWKVDNALQSGNSQESVTEQDSKDSTYSLSSTLTLSKADYEKHKVYACEVTHQGLSSPVTKSCNRGES

SEQ ID NO: 55 - protein, 103 aa, Artificial Sequence
  Anti-CD47 CH1 A129K
ASTKGPSVFPLKPSSCSTSGGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 56 - protein, 103 aa, Artificial Sequence
  Anti-CD47 CH1 A129R
ASTKGPSVFPLRPSSCSTSGGTAALGCLVKDYFPEPVTVSWNSGALTSGVHTFPAVLQSSGLYSLSSVVTVPSSSLGTQTYICNVNHKPSNTKVDKKVEPKSS

SEQ ID NO: 57 - protein, 107 aa, Artificial Sequence
  Anti-CD47 CL S121D
RTVAAPSVFIFPPDDEQLKSGTASVVCLLNNFYPREAKVQWKVDNALQSGNSQESVTEQDSKDSTYSLSSTLTLSKADYEKHKVYACEVTHQGLSSPVTKSCNRGES

SEQ ID NO: 58 - protein, 107 aa, Artificial Sequence
  Anti-CD47 CL S121E
RTVAAPSVFIFPPEDEQLKSGTASVVCLLNNFYPREAKVQWKVDNALQSGNSQESVTEQDSKDSTYSLSSTLTLSKADYEKHKVYACEVTHQGLSSPVTKSCNRGES

SEQ ID NO: 59 - protein, 119 aa, Artificial Sequence
  Anti-CLDN18.2 VH Q39K
EVQLVESGGGLVQPGGSLRLSCAASGFIFSSFGMHWVRKAPGKGLEWVAYISSGRSTMYYADSVKGRFTISRDNSKNTLYLQMNSLTAEDTAVYYCARGGFYGNSLDYWGQGTLVTVSS

SEQ ID NO: 60 - protein, 113 aa, Artificial Sequence
  Anti-CLDN18.2 VL Q38E
DIQMTQSPSSLSASVGDRVTITCKSSLSLLNSGNQKNYLTWYQEKPGKAPKLLIYWASTRESGVPSRFSGSGSGTDFTLTISSLQPEDFATYSCQNAYSYPLTFGQGTKVEIK

SEQ ID NO: 61 - protein, 119 aa, Artificial Sequence
  Anti-CLDN18.2 VH Q39E
EVQLVESGGGLVQPGGSLRLSCAASGFIFSSFGMHWVREAPGKGLEWVAYISSGRSTMYYADSVKGRFTISRDNSKNTLYLQMNSLTAEDTAVYYCARGGFYGNSLDYWGQGTLVTVSS

SEQ ID NO: 62 - protein, 113 aa, Artificial Sequence
  Anti-CLDN18.2 VL Q38K
DIQMTQSPSSLSASVGDRVTITCKSSLSLLNSGNQKNYLTWYQKKPGKAPKLLIYWASTRESGVPSRFSGSGSGTDFTLTISSLQPEDFATYSCQNAYSYPLTFGQGTKVEIK

SEQ ID NO: 63 - protein, 103 aa, Artificial Sequence
  Anti-CLDN18.2 CH1 G166K
ASTKGPSVFPLAPSSKSTSGGTAALGCLVKDYFPEPVTVSWNSGALTSKVHTFPAVLQSSGLYSLSSVVTVPSSSLGTQTYICNVNHKPSNTKVDKKVEPKSC

SEQ ID NO: 64 - protein, 107 aa, Artificial Sequence
  Anti-CLDN18.2 CL S114E
RTVAAPEVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 103 aa, Artificial Sequence
  Anti-CLDN18.2 CH1 G166E
ASTKGPSVFPLAPSSKSTSGGTAALGCLVKDYFPEPVTVSWNSGALTSEVHTFPAVLQSSGLYSLSSVVTVPSSSLGTQTYICNVNHKPSNTKVDKKVEPKSC

SEQ ID NO: 66 - protein, 107 aa, Artificial Sequence
  Anti-CLDN18.2 CL S114K
RTVAAPKVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 103 aa, Artificial Sequence; identical to SEQ ID NO: 10
  Anti-CLDN18.2 CH1 T187K

SEQ ID NO: 68 - protein, 107 aa, Artificial Sequence; identical to SEQ ID NO: 12
  Anti-CLDN18.2 CL D170E

SEQ ID NO: 69 - protein, 103 aa, Artificial Sequence
  Anti-CLDN18.2 CH1 T187E
ASTKGPSVFPLAPSSKSTSGGTAALGCLVKDYFPEPVTVSWNSGALTSGVHTFPAVLQSSGLYSLSSVVEVPSSSLGTQTYICNVNHKPSNTKVDKKVEPKSC

SEQ ID NO: 70 - protein, 107 aa, Artificial Sequence
  Anti-CLDN18.2 CL D170K
RTVAAPSVFIFPPSDEQLKSGTASVVCLLNNFYPREAKVQWKVDNALQSGNSQESVTEQDSKKSTYSLSSTLTLSKADYEKHKVYACEVTHQGLSSPVTKSFNRGEC

SEQ ID NO: 71 - protein, 103 aa, Artificial Sequence
  Anti-CLDN18.2 CH1 S131K
ASTKGPSVFPLAPKSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 72 - protein, 107 aa, Artificial Sequence
  Anti-CLDN18.2 CL P119E
RTVAAPSVFIFEPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 103 aa, Artificial Sequence
  Anti-CLDN18.2 CH1 A129K
ASTKGPSVFPLKPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 74 - protein, 107 aa, Artificial Sequence
  Anti-CLDN18.2 CL S121E
RTVAAPSVFIFPPEDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - protein, 103 aa, Artificial Sequence
  Anti-CLDN18.2 CH1 S131E
ASTKGPSVFPLAPESKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 76 - protein, 107 aa, Artificial Sequence
  Anti-CLDN18.2 CL P119K
RTVAAPSVFIFKPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 103 aa, Artificial Sequence
  Anti-CLDN18.2 CH1 A129E
ASTKGPSVFPLEPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 78 - protein, 107 aa, Artificial Sequence
  Anti-CLDN18.2 CL S121K
RTVAAPSVFIFPPKDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0124574 A1
(43) Pub. Date: Apr. 18, 2024

(54) Bispecific Antibodies with Charge Pairs and Uses Thereof

(71) Applicant: Phanes Therapeutics, Inc., San Diego, CA (US)

(72) Inventors: Jack Chongyang Li, San Diego, CA (US); Haiqun Jia, San Diego, CA (US); Hui Zou, San Diego, CA (US); Huiwen Wu, San Diego, CA (US); Minghan Wang, San Diego, CA (US)

(21) Appl. No.: 18/264,029

(22) PCT Filed: Jan. 28, 2022
(86) PCT No.: PCT/US2022/070394

§ 371 Date: Aug. 2, 2023

Related U.S. Application Data
(60) Provisional application No. 63/146,334, filed on Feb. 5, 2021.
(60) Provisional application No. 63/260,463, filed on Aug. 20, 2021.

(51) Int. Cl. C07K 16/28; C07K 16/18

(52) U.S. Cl. CPC C07K 16/2803; C07K 16/18; C07K 16/28; C07K 16/2809; C07K 2317/31; C07K 2317/522; C07K 2317/524; C07K 2317/526; C07K 2317/76

(57) ABSTRACT

Engineered bispecific antibodies with charge pairs introduced at the interface of CHI and CL alone or in combination with other charge pairs at the interface of VH and VL are described. Also described are anti-CD47/anti-CLDN18.2 and anti-CD3/anti-DLL3 bispecific antibodies and antigen-binding fragments thereof. Also described are nucleic acids encoding the bispecific antibodies, compositions comprising the bispecific antibodies, and methods of producing the bispecific antibodies and using the bispecific antibodies for treating or preventing diseases, such as cancer and/or associated complications.

### CROSS REFERENCE TO RELATED APPLICATIONS

This application claims priority to U.S. Provisional Application No. 63/146,334, filed on Feb. 5, 2021, and U.S. Provisional Application No. 63/260,463, filed on Aug. 20, 2021. Each disclosure is incorporated herein by reference in its entirety.

### FIELD OF THE INVENTION

This invention relates to engineered bispecific antibodies comprising designed charge residues in the interface between the heavy chain and the light chain. The invention relates to the charge pairs, the bispecific antibodies which contain the charge pairs, nucleic acids and expression vectors encoding the bispecific antibodies, recombinant cells containing the vectors, and compositions comprising the bispecific antibodies. Methods of screening and using charge pair designs that can differentiate the physical properties of the bispecific antibodies and their major impurities during production, making the bispecific antibodies, and methods of using the bispecific antibodies to treat diseases including cancer and/or associated complications are also provided.

### REFERENCE TO SEQUENCE LISTING SUBMITTED ELECTRONICALLY

This application contains a sequence listing, which is submitted electronically via EFS-Web as an ASCII formatted sequence listing with a file name “065799.35WO1 Sequence Listing” and a creation date of Dec. 10, 2021 and having a size of 80 kb. The sequence listing submitted via EFS-Web is part of the specification and is herein incorporated by reference in its entirety.

### BACKGROUND OF THE INVENTION

Antibodies (immunoglobulins) are naturally occurring proteins that play important roles in the immune system's function in defending the body against foreign objects such as bacteria and viruses. Antibodies in their native structure exist as a Y-shaped protein, consisting of two arms with each containing an identical heavy chain (HC) and an identical light chain (LC). The heavy chain contains one variable region (VH) and three constant regions (CH1, CH2 and CH3, respectively) that are arranged in the order of VH, CH1, CH2 and CH3 from the N-terminus to the C-terminus, and the light chain contains one variable region (VL) and one constant region (CL) that are arranged in the order of VL and CL from the N-terminus to the C-terminus. The association of the heavy chain and the light chain in each arm is usually termed “pairing,” which involves VH, CH1, VL, and CL. The VH and VL physically interact with each other to form the binding domain of the antibody against its antigen so that the Y-shaped antibody has two identical binding domains with one on each arm against the same antigen, and therefore is bivalent and mono-specific, a typical characteristic of a monoclonal antibody (mAb).

As part of the antibody structure, CH1 and CL also physically interact with each other, which involves physical contacts as well as an interchain disulfide bond (also called “disulfide bridge”) formed by the free thiol groups of two native cysteines on CH1 and CL, respectively. The interchain disulfide bond helps stabilize the overall structure formed by the heavy chain and light chain on each arm. In addition, inner-chain disulfide bonds are also formed as part of the native antibody structure. The C-terminus of the heavy chains (CH2 and CH3) forms a tight structure, which is important for the bivalency of the native antibody.

Monoclonal antibodies (mAbs) have been an excellent protein therapeutic platform due to their high affinity binding to antigens, the long half-life in vivo, naturally occurring stable structure, the ability to activate the immune system against drug targets, and many other aspects. However, when therapeutic strategies require targeting two separate antigens, such as two tumor-specific antigens on the same cancer cell with one antibody, a mAb cannot serve the purpose. Under such a circumstance, a bispecific antibody is made to target two different antigens on the same cell, with one arm binding to the first antigen and the other arm binding to the second antigen. Although there is mono-valency for each antigen binding, the binding to both antigens on the same cell can compensate for the lost avidity due to loss of the bivalency against each antigen. Bispecific antibodies provide increased selectivity when compared with mAbs because they have higher binding to cells expressing both antigens than cells expressing only one of the antigens. This is especially important in reducing safety concerns when normal cells or tissues express one of the two antigens. Bispecific antibodies can target two pathways simultaneously when it binds to two different cell surface antigens or soluble ligands/proteins, which is another advantage over mAbs. Bispecific antibodies that bind to one antigen on one cell and another antigen on a second cell can be used as cell engagers (such as T cell engagers) to bring two cells together and trigger intended biological effects to achieve a therapeutic goal.

If a bispecific antibody is made from two mAbs, the product would contain two different arms from the two mAbs, respectively, with each arm having a unique heavy chain and a unique light chain. The expression of the bispecific antibody in production cells during the manufacturing process requires the expression of 4 different proteins: the two different heavy chains and the two different light chains. While the goal is to have each HC pair with its corresponding LC on each arm of the bispecific antibody during production, mispairings (the LC of one arm pairs with the HC of the other arm) usually occur and generate unwanted products, making it challenging to produce and isolate the intended bispecific antibody product. Several approaches have been employed to improve the manufacturing aspect of the bispecific antibodies. One example is to identify a common light chain through protein engineering. However, domain swaps and many mutations used in protein engineering could significantly change the native antibody structure, which can increase the risk of aggregation and/or reduce the stability.

T cell engagers are multi-specific antibodies or antigen-binding fragments consisting of at least two binding domains with one domain binding to a tumor-associated antigen (TAA) expressed on the surface of a cancer cell, and the other domain binding to a T cell surface molecule to activate the T cell. Although various T cell binding domains have been used as the activating component, anti-CD3 binding domains have been widely used as T cell engagers. Anti-CD3 bispecific antibodies have been used as T cell-engaging immunotherapeutic agents for recruiting T cells to tumor cells to facilitate cancer killing.

### BRIEF SUMMARY OF THE INVENTION

In one general aspect, the invention relates to isolated bispecific antibodies or antigen-binding fragments thereof comprising:
a. a first heavy chain, H1;
b. a second heavy chain, H2;
c. a first light chain, L1; and
d. a second light chain, L2;
   wherein H1 and L1 form a first arm comprising a first antigen-binding domain that specifically binds a first antigen, preferably a first antigen of human origin, and wherein H2 and L2 form a second arm comprising a second antigen-binding domain that specifically binds a second antigen, preferably a second antigen of human origin, wherein
(a) H1 and H2 each comprises a CH1 region of human IgG1, IgG2, IgG3, or IgG4; and
(b) L1 and L2 each comprises a CL region of a human kappa light chain or a human lambda light chain;
   wherein H1L1 and H2L2 each comprise a charge pair selected from the group consisting of the following amino acid substitutions:
(1) G166D/E in CH1 of H1 and S114K/R in CL of L1, respectively, and G166K/R in CH1 of H2 and S114D/E in CL of L2, respectively;
(2) T187D/E in CH1 of H1 and D/N170K/R in CL of L1, respectively, and T187K/R in CH1 of H2 and DN170D/E in CL of L2, respectively;
(3) S131D/E in CH1 of H1 and P119K/R in CL of L1, respectively, and S131K/R in CH1 of H2 and P119D/E in CL of L2, respectively;
(4) A129D/E in CH1 of H1 and S121K/R in CL of L1, respectively, and A129K/R in CH1 of H2 and S121D/E in CL of L2, respectively;
(5) G166D/E in CH1 of H2 and S114K/R in CL of L2, respectively, and G166K/R in CH1 of H1 and S114D/E in CL of L1, respectively;
(6) T187D/E in CH1 of H2 and D/N170K/R in CL of L2, respectively, and T187K/R in CH1 of H1 and D/N170D/E in CL of L1, respectively;
(7) S131D/E in CH1 of H2 and P119K/R in CL of L2, respectively, and S131K/R in CH1 of H1 and P119D/E in CL of L1, respectively; or
(8) A129D/E in CH1 of H2 and S121K/R in CL of L2, respectively, and A129K/R in CH1 of H1 and S121D/E in CL of L1, respectively.

In certain embodiments, the two heavy chains H1 and H2 each comprise a VH region, a CH1 region, and a Fc region (containing CH2 and CH3 regions), wherein the VH regions have different amino acid sequences. In certain embodiments, the two heavy chains H1 and H2 each comprise a VH region, a CH1 region, and a Fc region (containing CH2 and CH3 regions), wherein the CH1 regions have different amino acid sequences. In certain embodiments, the two heavy chains H1 and H2 each comprise a VH region, a CH1 region, and a Fc region (containing CH2 and CH3 regions), wherein the Fc regions have different amino acid sequences. In certain embodiments, the two light chains L1 and L2 each comprise a VL region and a CL region, wherein the VL regions have different amino acid sequences. In certain embodiments, the two light chains L1 and L2 each comprise a VL region and a CL region, wherein the CL regions have different amino acid sequences.

In certain embodiments, H1 and H2 form a heterodimer.

In certain embodiments, a negatively charged amino acid (D or E) is introduced at G166, T187, S131, or A129 in CH1 of H1 (with a positively charged amino acid introduced at the corresponding position in CL of L1 as described above) and a positively charged amino acid (K or R) is introduced at the corresponding residue in CH1 of H2 (with a negatively charged amino acid introduced at the corresponding position in CL of L2 as described above), the VH region of H1 and the VL region of L1 have a Q39E and a Q38K substitution mutation, respectively, and the VH region of H2 and the VL region of L2 have a Q39K and a Q38E substitution mutation, respectively; or a positively charged amino acid (K or R) is introduced at G166, T187, S131, or A129 in CH1 of H1 (with a negatively charged amino acid introduced at the corresponding position in CL of L1 as described above) and a negatively charged amino acid (D or E) is introduced at the corresponding residue in CH1 of H2 (with a positively charged amino acid introduced at the corresponding position in CL of L2 as described above), the VH region of H1 and the VL region of L1 have a Q39K and a Q38E substitution mutation, respectively, and the VH region of H2 and the VL region of L2 have a Q39E and a Q38K substitution mutation, respectively.

In certain embodiments, the isolated bispecific antibody or antigen-binding fragment thereof comprises the CH1 and CL regions of one of the two arms comprising amino acid substitutions at an amino acid residue corresponding to the amino acid position of SEQ ID NO:17, 18, 19, or 20 for CH1 and SEQ ID NO:21 or 22 for CL, wherein the amino acid substitutions in the CH1 and CL regions are selected from:
(1) K133C and C220X in CH1, and F209C and C214X in CL;
(2) R133C and C131X in CHL and F209C and C214X in CL;
(3) R133C and C131X in CHL and V209C and C214X in CL; or
(4) K133C and C220X in CHL and V209C and C214X in CL;
   wherein X is selected from S, A or G.

In certain embodiments, the isolated bispecific antibody or antigen-binding fragment thereof comprises an anti-CD47 antibody or antigen-binding fragment arm and an anti-TAA antibody or antigen-binding fragment arm thereof, wherein CD47 and the TAA are expressed on the same cell, and is capable of specific binding to both CD47 and the TAA, preferably human CD47 and TAA. The isolated anti-CD47 antibody or antigen-binding fragment thereof can, for example, comprise the VH, CHL VL, and CL comprising the amino acid sequences of SEQ ID: 23, 28, 24 and 29, respectively.

In certain embodiments, the isolated bispecific antibody or antigen-binding fragment comprises a first antigen-binding domain that specifically binds CD47, preferably human CD47, and a second antigen-binding domain that specifically binds claudin 18.2 (CLDN18.2), preferably human CLDN18.2.

In certain embodiments, the anti-CD47 antigen-binding domain comprises the VH, CHL VL, and CL comprising the amino acid sequences of:
(1) SEQ ID NOs: 1, 27, 2 and 29, respectively;
(2) SEQ ID NOs: 1, 28, 2 and 29, respectively;
(3) SEQ ID NOs: 1, 27, 2 and 30, respectively;
(4) SEQ ID NOs: 1, 28, 2 and 30, respectively;
(5) SEQ ID NOs: 1, 31, 2 and 33, respectively;
(6) SEQ ID NOs: 1, 32, 2 and 33, respectively;
(7) SEQ ID NOs: 1, 31, 2 and 34, respectively;
(8) SEQ ID NOs: 1, 32, 2 and 34, respectively;
(9) SEQ ID NOs: 1, 35, 2 and 37, respectively;
(10) SEQ ID NOs: 1, 36, 2 and 37, respectively;
(11) SEQ ID NOs: 1, 35, 2 and 38, respectively;
(12) SEQ ID NOs: 1, 36, 2 and 38, respectively;
(13) SEQ ID NOs: 1, 39, 2 and 41, respectively;
(14) SEQ ID NOs: 1, 40, 2 and 41, respectively;
(15) SEQ ID NOs: 1, 39, 2 and 42, respectively;
(16) SEQ ID NOs: 1, 40, 2 and 42, respectively;
(17) SEQ ID NOs: 1, 43, 2 and 45, respectively;
(18) SEQ ID NOs: 1, 44, 2 and 45, respectively;
(19) SEQ ID NOs: 1, 43, 2 and 46 respectively;
(20) SEQ ID NOs: 1, 44, 2 and 46, respectively;
(21) SEQ ID NOs: 1, 47, 2 and 49, respectively;
(22) SEQ ID NOs: 1, 48, 2 and 49, respectively;
(23) SEQ ID NOs: 1, 47, 2 and 50, respectively;
(24) SEQ ID NOs: 1, 48, 2 and 50, respectively;
(25) SEQ ID NOs: 1, 51, 2 and 53, respectively;
(26) SEQ ID NOs: 1, 52, 2 and 53, respectively;
(27) SEQ ID NOs: 1, 51, 2 and 54, respectively;
(28) SEQ ID NOs: 1, 52, 2 and 54, respectively;
(29) SEQ ID NOs: 1, 55, 2 and 57, respectively;
(30) SEQ ID NOs: 1, 56, 2 and 57, respectively;
(31) SEQ ID NOs: 1, 55, 2 and 58, respectively;
(32) SEQ ID NOs: 1, 56, 2 and 58, respectively;
(33) SEQ ID NOs: 23, 27, 24 and 29, respectively;
(34) SEQ ID NOs: 23, 28, 24 and 29, respectively;
(35) SEQ ID NOs: 23, 27, 24 and 30, respectively;
(36) SEQ ID NOs: 23, 28, 24 and 30, respectively;
(37) SEQ ID NOs: 25, 31, 26 and 33, respectively;
(38) SEQ ID NOs: 25, 32, 26 and 33, respectively;
(39) SEQ ID NOs: 25, 31, 26 and 34, respectively;
(40) SEQ ID NOs: 25, 32, 26 and 34, respectively;
(41) SEQ ID NOs: 23, 35, 24 and 37, respectively;
(42) SEQ ID NOs: 23, 36, 24 and 37, respectively;
(43) SEQ ID NOs: 23, 35, 24 and 38, respectively;
(44) SEQ ID NOs: 23, 36, 24 and 38, respectively;
(45) SEQ ID NOs: 25, 39, 26 and 41, respectively;
(46) SEQ ID NOs: 25, 40, 26 and 41, respectively;
(47) SEQ ID NOs: 25, 39, 26 and 42, respectively;
(48) SEQ ID NOs: 25, 40, 26 and 42, respectively;
(49) SEQ ID NOs: 23, 43, 24 and 45, respectively;
(50) SEQ ID NOs: 23, 44, 24 and 45, respectively;
(51) SEQ ID NOs: 23, 43, 24 and 46 respectively;
(52) SEQ ID NOs: 23, 44, 24 and 46, respectively;
(53) SEQ ID NOs: 25, 47, 26 and 49, respectively;
(54) SEQ ID NOs: 25, 48, 26 and 49, respectively;
(55) SEQ ID NOs: 25, 47, 26 and 50, respectively;
(56) SEQ ID NOs: 25, 48, 26 and 50, respectively;
(57) SEQ ID NOs: 23, 51, 24 and 53, respectively;
(58) SEQ ID NOs: 23, 52, 24 and 53, respectively;
(59) SEQ ID NOs: 23, 51, 24 and 54, respectively;
(60) SEQ ID NOs: 23, 52, 24 and 54, respectively;
(61) SEQ ID NOs: 25, 55, 26 and 57, respectively;
(62) SEQ ID NOs: 25, 56, 26 and 57, respectively;
(63) SEQ ID NOs: 25, 55, 26 and 58, respectively; or
(64) SEQ ID NOs: 25, 56, 26 and 58, respectively.

In certain embodiments, the isolated bispecific antibody or antigen-binding fragment is an anti-CD47/anti-CLDN18.2 bispecific antibody, wherein the first antigen-binding domain comprises the VH, CH1, VL, and CL, and the second antigen-binding domain comprises the VH, CH1, VL, and CL, comprising the amino acid sequences of:
(1) SEQ ID: 1, 28, 2, 29, 3, 63, 4 and 64, respectively;
(2) SEQ ID: 1, 36, 2, 37, 3, 67, 4 and 68, respectively;
(3) SEQ ID: 1, 44, 2, 45, 3, 71, 4 and 72, respectively;
(4) SEQ ID: 1, 52, 2, 53, 3, 73, 4 and 74, respectively;
(5) SEQ ID: 1, 31, 2, 34, 3, 65, 4 and 66, respectively;
(6) SEQ ID: 1, 39, 2, 42, 3, 69, 4 and 70, respectively;
(7) SEQ ID: 1, 47, 2, 50, 3, 75, 4 and 76, respectively;
(8) SEQ ID: 1, 55, 2, 58, 3, 77, 4 and 78, respectively;
(9) SEQ ID: 23, 28, 24, 29, 59, 63, 60 and 64, respectively;
(10) SEQ ID: 23, 36, 24, 37, 59, 67, 60 and 68, respectively;
(11) SEQ ID: 23, 44, 24, 45, 59, 71, 60 and 72, respectively;
(12) SEQ ID: 23, 52, 24, 53, 59, 73, 60 and 74, respectively;
(13) SEQ ID: 25, 31, 26, 34, 61, 65, 62 and 66, respectively;
(14) SEQ ID: 25, 39, 26, 42, 61, 69, 62 and 70, respectively;
(15) SEQ ID: 25, 47, 26, 50, 61, 75, 62 and 76, respectively; or
(16) SEQ ID: 25, 55, 26, 58, 61, 77, 62 and 78, respectively.

In certain embodiments, the isolated bispecific antibody or antigen-binding fragment thereof comprises an anti-immune cell modulator (ICM) antibody or antigen-binding fragment arm thereof and is capable of specific binding to ICM, preferably a human ICM. The ICM can, for example, be selected from the group consisting of CD3, CD16, CD27, CD28, CD40, CD122, NKp46, OX40, 4-1BB, GITR, ICOS, CTLA-4, PD-1, LAG-3, TIM-3, VISTA, SIGLEC7, SIGLEC9, KIR, BTLA, B7-H3, and other cell surface immune regulatory molecules.

In certain embodiments, the anti-ICM antibody or antigen-binding fragment thereof is an anti-CD3 antibody or antigen-binding fragment thereof and is capable of specific binding to CD3, preferably human CD3. The isolated anti-CD3 antibody or antigen-binding fragment thereof can, for example, comprise the VH, CH1, VL, and CL comprising the amino acid sequences of SEQ ID: 9, 10, 11 and 12, respectively.

In certain embodiments, the isolated bispecific antibody or antigen-binding fragment is an anti-CD3/anti-DLL3 bispecific antibody, wherein the first antigen-binding domain specifically binds CD3, preferably human CD3, and the second antigen-binding domain specifically binds DLL3, preferably human DLL3.

In certain embodiments, the isolated bispecific antibody or antigen-binding fragment is an anti-CD3/anti-DLL3 bispecific antibody, wherein the first antigen-binding domain comprises the VH, CH1, VL, and CL comprising the amino acid sequences of SEQ ID: 9, 10, 11 and 12, respectively, and a second antigen-binding domain comprises the VH, CHL VL, and CL comprising the amino acid sequences of SEQ ID: 13, 14, 15 and 16, respectively.

Also provided are isolated nucleic acids encoding the isolated bispecific antibodies or antigen-binding fragments thereof of the invention disclosed herein.

Also provided are vectors comprising the isolated nucleic acids encoding the bispecific antibodies or antigen-binding fragments thereof of the invention disclosed herein.

Also provided are host cells comprising the vectors comprising the isolated nucleic acids encoding the bispecific antibodies or antigen-binding fragments thereof of the invention disclosed herein.

In certain embodiments, provided is a pharmaceutical composition comprising the isolated bispecific antibodies or antigen-binding fragments thereof of the invention and a pharmaceutically acceptable carrier.

Also provided are methods of targeting CD47 and a TAA (such as, for example, CLDN18.2) that are both expressed on a cancer cell surface in a subject in need thereof by activating macrophage-mediated phagocytosis of the cancer cell, comprising administering to the subject a pharmaceutical composition of the invention.

Also provided are methods of targeting DLL3 that is expressed on a cancer cell surface in a subject in need thereof, by engaging T cells, comprising administering to the subject a pharmaceutical composition of the invention.

Also provided are methods of targeting one or two antigens expressed on a cancer cell surface in a subject in need thereof including using CD47 blockade induced activation of macrophage-mediated phagocytosis or CD3-mediated T cell activation, comprising administering to the subject a pharmaceutical composition of the invention.

Also provided are methods of treating cancer in a subject in need thereof, comprising administering to the subject the pharmaceutical compositions of the invention. The cancer can be any liquid or solid cancer, for example, it can be selected from, but not limited to, a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Also provided are methods of producing the isolated bispecific antibody or antigen-binding fragment thereof of the invention, comprising culturing a cell comprising a nucleic acid encoding the bispecific antibody or antigen-binding fragment thereof under conditions to produce the bispecific antibody or antigen-binding fragment thereof, and recovering the bispecific antibody or antigen-binding fragment thereof from the cell or culture.

Also provided are methods of producing a pharmaceutical composition comprising the isolated bispecific antibody or antigen-binding fragment thereof of the invention, comprising combining the bispecific antibody or antigen-binding fragment thereof with a pharmaceutically acceptable carrier to obtain the pharmaceutical composition.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows the schematic structure of a bispecific antibody (bsAb) with the mAb 1 arm and the mAb 2 arm. Both arms have different heavy chain VH and light chain VL regions; the heavy chains (HCs) and light chains (LCs) of the bispecific antibody are on the backbone of IgG1 and kappa, respectively, for the purpose of presenting an example. Knob in the hole (KiH) mutations are introduced in the CH3 regions of both HCs to promote heterodimer formation. In addition, a cysteine residue was introduced to each of the two CH3 regions, respectively, to promote the formation of an interchain disulfide bond to stabilize the heterodimer. H1 and L1 are the heavy chain and light chain of the mAb 1 arm, respectively, and H2 and L2 are the heavy chain and light chain of the mAb 2 arm, respectively.

FIGS. 2A-2F show sequences of various antibody components. FIG. 2A shows the alignment of CH1 regions of human IgG1 (SEQ ID NO:17), IgG2 (SEQ ID NO:18), IgG3 (SEQ ID NO:19), and IgG4 (SEQ ID NO:20). FIG. 2B shows the alignment of CL regions of human kappa (SEQ ID NO:21) and lambda (SEQ ID NO:22) light chains. FIGS. 2C-2D show the VH (FIG. 2C) and VL (FIG. 2D) sequences of an anti-CD47 mAb (VH: SEQ ID NO:1; VL: SEQ ID NO:2) and an anti-CLDN18.2 mAb (VH: SEQ ID NO:3; VL: SEQ ID NO:4). FIGS. 2E-2F show the VH (FIG. 2E) and VL (FIG. 2F) sequences of an anti-CD3 mAb (VH: SEQ ID NO:5; VL: SEQ ID NO:6) and an anti-DLL3 mAb (VH: SEQ ID NO:7; VL: SEQ ID NO:8). The CDR regions determined by Kabat method are highlighted. * represents sites of known allelic variations.

FIGS. 3A-3Q show the analyses of bispecific antibodies constructed with various charge pairs with standard transfection ratio. FIGS. 3A-3B show the result of a bridging ELISA assay with Protein A purified samples from the media of ExpiCHO-S cells transfected with the indicated bispecific antibody constructs. FIGS. 3C-3N show the CEX-HPLC (cation exchange chromatography on high performance liquid chromatography) analyses of Protein A purified samples from the media of ExpiCHO-S cells transfected with the indicated bispecific antibody constructs or transfected with major impurity standards for the bispecific antibody constructs. FIGS. 3O-3Q show the liquid chromatography/mass spectrometry (LC/MS) analyses of Protein A purified samples from the media of ExpiCHO-S cells transfected with the indicated bispecific antibody constructs. WT, bispecific antibody with anti-CD47 (mAb 1 arm) and anti-CLDN18.2 (mAb 2 arm) arms as depicted in FIG. 1; the other bispecific antibodies contain modifications to the WT construct as described in Table 3. The major impurity standards for each bispecific construct were generated and analyzed on CEX-HPLC along with that bispecific antibody: anti-CD47 knob homodimer/half mol., Protein A purified sample from the media of ExpiCHO-S cells transfected with the anti-CD47 HC and LC of the bispecific antibody design; anti-CLDN18.2 hole homodimer/half mol., Protein A purified sample from the media of ExpiCHO-S cells transfected with the anti-CLDN18.2 HC and LC of the bispecific antibody design; 2×anti-CD47 LC mismatch (also called 2×anti-CD47 LC), Protein A purified sample from the media of ExpiCHO-S cells transfected with anti-CD47 HC and LC and anti-CLDN18.2 HC of the bispecific antibody design; 2×anti-CLDN18.2 LC mismatch (also called 2×anti-CLDN18.2 LC), Protein A purified sample from the media of the ExpiCHO-S cells transfected with anti-CLDN18.2 HC and LC and anti-CD47 HC of the bispecific antibody design.

FIGS. 4A-4L show the analyses of bispecific antibodies constructed with various “EK” charge pairs with biased DNA ratio for transfection. FIG. 4A shows the result of a bridging ELISA assay with Protein A purified samples from the media of ExpiCHO-S cells transfected at a biased DNA ratio with the indicated bispecific antibody constructs. FIG. 4B shows the CEX-HPLC analyses of the Protein A purified samples. FIGS. 4C-4D show the LC/MS analyses of Protein A purified samples from the media of ExpiCHO-S cells transfected with the indicated bispecific antibody constructs with biased DNA ratio. FIGS. 4E-4F show the CEX-FPLC (cation exchange chromatography on fast protein liquid chromatography) analyses of the indicated Protein A purified bispecific antibody samples using a linear pH gradient. FIG. 4G shows the result of a bridging ELISA assay of the samples from the different peaks in FIGS. 4E-4F. FIGS. 4H-4L show the LC/MS analyses of the peaks from the CEX-FPLC chromatography in FIGS. 4E-4F.

FIGS. 5A-5F show the analyses of bispecific antibodies constructed with various “KE” charge pairs with standard DNA ratio for transfection. FIG. 5A shows the CEX-FPLC analysis of the indicated Protein A purified bispecific antibody samples using a linear pH gradient. FIG. 5B shows the result of a bridging ELISA assay of the samples from the different peaks in FIGS. 5A. FIGS. 5C-5F show the LC/MS analyses of the peaks from the CEX-FPLC chromatography in FIG. 5A.

FIGS. 6A-6F show the stepwise CEX-FPLC purification and analyses of bispecific antibodies with the “EK” charge pairs. The Protein A purified samples from the media of ExpiCHO-S cells transfected at biased DNA ratio with the different bispecific antibody constructs were analyzed on CEX-FPLC, and an optimized stepwise purification method was used to purify each bispecific antibody (FIG. 6A). The sample from each peak of the stepwise CEX-FPLC in FIG. 6A was analyzed in a bridging ELISA assay for bispecific activity (FIG. 6B) and the purity of each peak was also analyzed using LC/MS (FIGS. 6C-6F).

FIGS. 7A-7K show the analyses of bispecific antibodies constructed with various “ekEK” or “keKE” charge pairs. FIG. 7A shows the result of a bridging ELISA assay with Protein A purified samples from the media of ExpiCHO-S cells transfected with the indicated bispecific antibody constructs. FIGS. 7B-7I show the CEX-HPLC analyses of Protein A purified samples from the media of ExpiCHO-S cells transfected with the indicated bispecific antibody constructs or their respective major impurity standards. FIGS. 7J-7K show the LC/MS analyses of Protein A purified samples from the media of ExpiCHO-S cells transfected with the indicated bispecific antibody constructs.

FIGS. 8A-8L show the CEX-FPLC analyses of the indicated Protein A purified bispecific antibodies with the “ekEK” or “keKE” charge pairs and the results of bridging ELISA and LC/MS assays of each peak. FIGS. 8A and 8G show the CEX-FPLC analyses of the “ekEK” and “keKE” bispecific antibodies, respectively, using a linear pH gradient. FIGS. 8B and 8H show the results of a bridging ELISA assay of the samples from the different peaks in FIGS. 8A and 8G, respectively. FIGS. 8C-8F show the LC/MS analyses of the peaks from the CEX-FPLC chromatography in FIG. 8A. FIGS. 8I-8L show the LC/MS analyses of the peaks from the CEX-FPLC chromatography in FIG. 8G.

FIGS. 9A-9J show the stepwise CEX-FPLC purification of the indicated Protein A purified bispecific antibodies with the charge pairs and the results of bridging ELISA and LC/MS assays of each peak. The Protein A purified samples from the media of ExpiCHO-S cells transfected with the different bispecific antibody constructs were analyzed on CEX-FPLC, and an optimized stepwise purification method was used to purify each bispecific antibody (FIGS. 9A and 9E). The sample from each collectable peak of the stepwise CEX-FPLC in FIGS. 9A and 9E was analyzed in a bridging ELISA assay for bispecific activity (FIGS. 9B and 9F) and the purity of each peak was also analyzed using LC/MS (FIGS. 9C-9D and 9G-9J).

FIGS. 10A-10C show the purity of the anti-CD3/anti-DLL3 bispecific antibody bsAb_33 using different analytical methods. FIG. 10A is the HIC (hydrophobic interaction chromatography) HPLC analysis of purified bsAb_33 with several impurity standards for comparison; FIG. 10B is the strong cation exchange (SCX) HPLC analysis of purified bsAb_33 with several impurity standards for comparison; FIG. 10C is the SEC (size exclusion chromatography) HPLC analysis of purified bsAb_33.

FIGS. 11A-11B show the assay results using the anti-CD3/anti-DLL3 bispecific antibody bsAb_33. FIG. 11A shows the cross-linking of SHP-77 cells (expressing DLL3) and Jurkat cells (expressing CD3) by the anti-CD3/anti-DLL3 bispecific antibody bsAb_33. FIG. 11B shows the activation of reporter Jurkat cells (expressing CD3) in the presence of SHP-77 cells (expressing DLL3) mediated by the anti-CD3/anti-DLL3 bispecific antibody bsAb_33. The anti-DLL3 blocking mAb is the mAb version of the anti-DLL3 arm of bsAb_33; the anti-CD3 blocking mAb is the mAb version of the anti-CD3 arm of bsAb_33.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The words “right,” “left,” “lower,” and “upper” designate directions in the drawings to which reference is made.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

As used herein, the terms “different heavy chains” or “different light chains” as used throughout the specification and claims, indicate that the heavy chains or the light chains have sequences that are not identical to each other.

The terms “identical” or percent “identity,” in the context of two or more nucleic acids or polypeptide sequences (e.g., bispecific antibodies, anti-CD3 antibodies, anti-DLL3 antibodies, anti-CD47 antibodies, anti-CLDN18.2 antibodies, anti-CD3/anti-DLL3 bispecific antibodies, anti-CD47/anti-CLDN18.2 bispecific antibodies, DLL3 polypeptides and polynucleotides that encode them, CD3 polypeptides and polynucleotides that encode them, CD47 polypeptides and polynucleotides that encode them, and CLDN18.2 polypeptides and polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected with a nucleic acid molecule of the invention. In another embodiment, a “host cell” is a progeny or potential progeny of such a transfected cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed bispecific antibody can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

As used herein, the term “CD47” refers to a multi-spanning transmembrane receptor belonging to the immunoglobulin superfamily, which has been indicated to be involved in multiple cellular process, including cell migration, adhesion, and T cell function. CD47, also known as integrin-associated protein (TAP), ovarian cancer antigen (OA3), Rh-related antigen, and MER6, was originally identified as a tumor antigen on human ovarian cancer and was subsequently shown to be expressed on multiple human tumor types, including both hematologic and solid tumors. The interaction between CD47 and signal regulatory protein alpha (SIRPa), an inhibitory protein expressed on macrophages, prevents phagocytosis of CD47-expressing cells. CD47 is additionally expressed at low levels on virtually all non-alignant cells. The term “human CD47” refers to a CD47 originated from a human. An exemplary amino acid sequence of a human CD47 is represented in GenBank Accession No. NP_001768.1.

As used herein, the term “CLDN18.2” refers to claudin 18 variant 2, claudin 18.2, claudin-18.2 or claudin-18a2.1, which belongs to the claudin family of transmembrane proteins. CLDN18.2 is specifically expressed on the surface of epithelial cells in stomach (Niimi et al., Mol Cell Biol. 2001; 21:7380-7390) and becomes one of the major structural components of the tight junction between the epithelial cells (Sahin et al., Physiol Rev. 2013; 93:525-569). The term “human CLDN18.2” refers to a CLDN18.2 originated from a human. An exemplary amino acid sequence of a human CLDN18.2 is represented in GenBank Accession No. AAL15637.1.

As described herein, the term “DLL3” refers to Delta like canonical Notch ligand 3 (DLL3), also known as delta like 3 or delta like protein 3, which is required for somite segmentation during early development (Dunwoodie et al., Development 129:1795-806 (2002)). Unlike the mammalian Notch family ligands DLL1, DLL4, JAG1, and JAG2 which all activate Notch receptor signaling in trans (Ntziachristos et al., Cancer Cell 25(3):318-34 (2014)), DLL3 is predominantly localized in the Golgi apparatus and is unable to activate Notch signaling (Chapman et al., Hum Mol Genet 20(5):905-16 (2011) and Geffers et al., J Cell Biol 178(3):465-76 (2007)). During normal development, DLL3 inhibits both cis- and trans-acting Notch pathway activation by interacting with Notch and DLL1 (Chapman et al., Hum Mol Genet 20(5):905-16(2011)). DLL3 is normally either absent or present at very low levels in adult normal tissues except brain, but is overexpressed in lung cancer, testicular cancer, glioma and melanoma samples (Uhlen et al., Science 357(6352): eaan2507 (2017)). Furthermore, DLL3 is detectable on the surface of small cell lung cancer (SCLC) and large cell neuroendocrine carcinoma (LCNEC) tumor cells (Saunders et al., Sci Trans! Med 7(302):302ra136 (2015) and Sharma et al., Cancer Res 77(14):3931-41 (2017)), making it a potential target of monoclonal antibodies for cancer therapy. Therefore, an anti-DLL3 monoclonal antibody could be used to specifically target DLL3-expressing tumor cells and serve as a potential anti-cancer therapeutic. The term “human DLL3” refers to a DLL3 originated from a human. An exemplary amino acid sequence of a human DLL3 is represented in GenBank Accession No. NP_058637.1.

As described herein, the term “CD3” refers to Cluster of Differentiation 3, which is a multi-subunit protein complex that functions as the co-receptor to T cell receptor (TCR) (Dong et al., Nature 573(7775):546-552 (2019)). Binding of TCR to peptide-MHC (pMHC) on the surface of the target cells induces the clustering of the TCR-CD3 complex and activates the intracellular signaling mediated by the chain of CD3 (Annu Rev Immunol. 27:591-619 (2009)). CD3 is required for the activation of T-cells and its pMHC-independent activation by therapeutics, such as in CAR-T-cells and by CD3-based T cell engagers, is highly effective in mobilizing T cells to kill tumor cells (Brown and Mackall, Nat Rev Immunol 19(2):73-74 (2019) and Clynes and Desjarlais, Annu Rev Med 70:437-450 (2019)). An exemplary amino acid sequence of a human CD3 epsilon subunit is represented in GenBank Accession No. NP_000724.1.

A “tumor-associated antigen (TAA),” as described herein, refers to any cell surface peptide and/or antigen or a combination of a cell surface peptide and/or antigen and its post-translational modifying moiety (such as glycosylation) that are present at a higher level in tumor than in normal tissues. Some of the tumor-associated antigens present specifically in tumors are also known as tumor-specific antigens (TSAs). Examples of tumor-associated antigens are viral proteins encoded by oncogenic viruses; mutated oncoproteins or tumor suppressors; normal proteins overexpressed on and/or in tumor cells; post-translational modifications of cell surface proteins; oncofetal proteins, whose expression are normally restricted in development stages but not in adult tissues; and cell-type specific proteins, whose expression are limited to unessential tissues.

An “immune cell modulator (ICM),” as described herein, refers to any cell surface molecule such as a protein that is expressed on the surface of immune cells and regulate the function of the immune cells. The ICMs include stimulatory molecules and inhibitory molecules. A stimulatory ICM can mediate the activation of the immune cells when a specific antibody or antigen-binding fragment with certain characteristics specifically binds to the stimulatory ICM. An inhibitory ICM suppresses the activity of the immune cell upon binding by a ligand/interacting partner, which can be blocked by a specific antibody or antigen-binding fragment with certain characteristics leading to the activation of the immune cells. These immune cells can be T cells, NK cells, macrophages or other types of cells of the immune system. Examples of ICMs include, but are not limited to, CD3, CD16, CD27, CD28, CD40, CD122, NKp46, OX40, 4-1BB, GITR, ICOS, CTLA-4, PD-1, LAG-3, TIM-3, VISTA, SIGLEC7, SIGLEC9, KIR, BTLA, and B7-H3.

As used herein the term “complete block” or “complete blockade” refers to the complete inhibition of a target antigen (e.g., an ICM, such as CD3) binding to the target antigen-binding domain (e.g., a monoclonal or bispecific antibody or antigen-binding fragment thereof). The complete inhibition of target antigen-binding means that there is no binding (e.g., 0% binding) of the target antigen to the target antigen-binding domain.

As used herein the term “partial block” or “partial blockade” refers to an incomplete inhibition of a target antigen (e.g., an ICM, such as CD3) binding to the target antigen-binding domain (e.g., a monoclonal or bispecific antibody or antigen-binding fragment thereof). The incomplete inhibition of target antigen-binding means that there is at least some binding (e.g., 1% to 99% binding) of the target antigen to the target antigen-binding domain.

### Antibodies and Bispecific Antibodies

The invention generally relates to isolated bispecific antibodies comprising charge mutations at the CH1 and CL interface in each arm to improve cognate chain pairing preference and/or modify and differentiate the physical properties of the bispecific antibody itself and the mis-paired impurities to facilitate purification using cation exchange chromatography.

Bispecific antibodies formed with two different heavy chains (HCs) and light chains (LCs) are difficult to produce due to the propensity of incorrect pairing of the two heavy chains and the two light chains, which results in the production of unwanted products that are difficult to eliminate in the manufacturing process; even when the unwanted products from mispairing can be eliminated during purification, the mispairing reduces the production efficiency for the intended bispecific antibody product. By combining the “shifted interchain disulfide bond” in one arm strategy (PCT/US2020/063066, filed on Dec. 3, 2020, which is incorporated by reference herein in its entirety) with charge pairs, and concurrently introducing knob in the hole and disulfide bond-forming cysteine mutations in the Fc regions, the heterodimeric bispecific antibody can be produced with improved efficiency and ease of purification using conventional methods such as ion exchange chromatography and hydrophobic interaction chromatography. Production of such bispecific antibodies, including, but not limited to anti-CD47/anti-CLDN18.2 bispecific antibodies and anti-CD3/anti-DLL3 bispecific antibodies, can be carried out by co-expressing the two heavy chains and the two light chains.

The invention generally relates to isolated bispecific antibodies with charge pairs, or a combination of charge pairs, or combination of charge pairs with a shifted interchain disulfide bond on one arm while maintaining the native interchain disulfide bond on the second arm. In particular, the invention generally relates to bispecific antibodies against two antigens that are expressed on the same cancer cell, two antigens with one on the cancer cell and the other as a soluble ligand, or two antigens with one on the cancer cell and the other on a different cell such as an immune cell; nucleic acids and expression vectors encoding the bispecific antibodies; recombinant cells containing the vectors; and compositions comprising the bispecific antibodies. Methods of making the bispecific antibodies, and methods of using the bispecific antibodies to treat diseases, including cancer, are also provided. The bispecific antibodies of the invention possess one or more desirable functional properties, including but not limited to high-affinity binding to both antigens, high specificity to both antigens, the ability to induce effector-mediated tumor cell lysis, the ability to stimulate complement-dependent cytotoxicity (CDC), antibody-dependent phagocytosis (ADPC), and/or antibody-dependent cell-mediated cytotoxicity (ADCC) against cells expressing one or two antigens, the ability to mediate the recruitment of conjugated drugs, and the ability to inhibit tumor growth in subjects and animal models when administered alone or in combination with other anti-cancer therapies. The bispecific antibodies of the invention can be one with both arms binding to two different antigens on the same cell and mediate multiple biological effects. One of the two arms can be CD47 which can be blocked to induce macrophage-mediated phagocytosis. The bispecific antibodies of the invention can also be immune cell engagers, such as T cell engagers, with one arm binding to an antigen on cancer cells and the other arm binding to T cells to mediate T-cell dependent cancer cell killing. The oppositely charged amino acids introduced in the CH1 and CL regions of each arm of the bispecific antibody can enhance the correct cognate pairing of each heavy chain with its corresponding light chain (H1L1 and H2L2). The oppositely charged pair(s) can better differentiate the physical properties of the bispecific antibody and potential impurities expressed by cells in the production process, facilitating the purification and production of the intended bispecific antibody.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

Several systems are used for the numbering of amino acid residues in antibodies. The Kabat numbering method is a scheme based on variable regions of antibodies (Elvin A. Kabat et al., Sequences of Proteins of Immunological Interest 5th ed. (1991). The EU numbering system is widely used for the constant domains (including portions of the CH1, hinge, and the Fc) (Elvin A. Kabat et al., Sequences of Proteins of Immunological Interest 5th ed. (1991).

As used herein, the term an “isolated antibody” refers to an antibody which is substantially free of other antibodies having different antigenic specificities (e.g., an isolated antibody that specifically binds to DLL3 is substantially free of antibodies that do not bind to DLL3, an isolated antibody that specifically binds to CD3 is substantially free of antibodies that do not bind to CD3, a bispecific antibody that specifically binds to CD3 and DLL3 is substantially free of antibodies that do not bind to CD3 and DLL3). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that may be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)2, a Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdab), a scFv dimer (bivalent diabody), a multi-specific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and a Fd segment of the heavy chain. According to other particular embodiments, the antigen-binding fragment comprises Fab and F(ab′).

As used herein, the term “single-chain antibody” refers to a conventional single-chain antibody in the field, which comprises a heavy chain variable region and a light chain variable region connected by a short peptide of about 15 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often correspond to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multi-specific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., the different proteins (or different subunits of a multimeric protein). In an embodiment, a multi-specific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope.

As used herein, the term “charge pair” refers to a pair of amino acids with one having positive charge and the other having negative charge, which can be introduced by replacing native amino acid residues in the heavy chain CH1 region and the light chain CL region of the first arm of a bispecific antibody, respectively, and concurrently, the same pair of positive charge and negative charge amino acids can be introduced by replacing native amino acid residues in the light chain CL region and the heavy chain CH1 region of the second arm of the bispecific antibody, respectively. Alternatively, the positive charge and negative charge amino acids can be introduced by amino acid substitution to the VH region of the heavy chain and the VL region of the light chain of the first arm of a bispecific antibody, respectively, and concurrently, the same pair of positive charge and the negative charge amino acids can be introduced by amino acid substitution to the VL region of the light chain and the VH region of the heavy chain of the second arm, respectively. Amino acids used to form charge pairs usually include D/E (negative charge) and K/R (positive charge). Once introduced to the CH1/CL regions or VH/VL regions, the charge pair amino acids are in close proximity structurally and are expected to enhance the heavy chain/light chain interaction of the same arm through opposite charges and expel the mismatched heavy chain/light chain interaction (the mismatched heavy and light chains are from the two different arms) through the same charges. The resulting charge distribution of the introduced charge pair is as follows: H1 (CH1 positive charge)/L1 (CL negative charge)/H2 (CH1 negative charge)/L2 (CL positive charge) or H1 (CH1 negative charge)/L1 (CL positive charge)/H2 (CH1 positive charge)/L2 (CL negative charge). Multiple charge pairs can be combined and introduced to the CH1 and CL interface, with all positive charge amino acids introduced to CH1 and all negative charge amino acids to CL of the same arm, or vice versa, to satisfy the above distribution pattern. A similar approach can be applied to the VH/VL interface. Further, one or multiple charge pairs can also be introduced to the interface of VH and VL in combination with one or multiple charge pairs introduced to the CH1/CL interface — amino acids introduced to the same chain (either H1, L1, H2 or L2) usually have the same charge, and the resulting distribution of the introduced charge pairs is as follows: H1 (CH1 and VH positive charge)/L1 (CL and VL negative charge)/H2 (CH1 and VH negative charge)/L2 (CL and VL positive charge) or H1 (CH1 and VH negative charge)/L1 (CL and VL positive charge)/H2 (CH1 and VH positive charge)/L2 (CL and VL negative charge). The charge pair substitutions can also be combined with other modifications to further improve the cognate chain pairing preference (H1L1 and H2L2, respectively) and/or facilitate purification of the bispecific antibody using ion exchange chromatography and/or HIC. For example, in addition to the charge pair substitutions, the native interchain disulfide bond on one arm of the bispecific antibody can be shifted while the other arm has the native interchain disulfide bond (see, e.g., WO2021/126538, which is incorporated by reference herein in its entirety).

In describing the charge pairs, G166D/E represents substitution of G at position 166 (EU numbering) with D or E, in which case G166 is the knock-in site; D170D/E represents keeping D at position 170 or substitution of D at position 170 with E; all the other substitutions follow the same naming rule.

As used herein, an antibody that “specifically binds to CD3, DLL3, and combinations thereof” or that “specifically binds to CD47, CLDN18.2, and combinations thereof” refers to an antibody that binds to CD3, DLL3, and combinations thereof or an antibody that binds to CD47, CLDN18.2, and combinations thereof, preferably human CD3, human DLL3, human CD47, human CLDN18.2, and combinations thereof with a KD of 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system.

The smaller the value of the KD of an antibody, the higher affinity that the antibody binds to a target antigen.

As used herein the term “specific binding” refers to the significant binding of the target antigen to an antibody or antigen-binding fragment thereof as compared to a control antigen, and/or the significant binding of the target antigen to an antibody or antigen-binding fragment thereof as compared to a control antibody or antigen-binding fragment, wherein the control antigen is different from the target antigen by sequence and/or structure comparison, and the control antibody or antigen-binding fragment significantly and selectively binds only to its corresponding antigen that is different from the target antigen by sequence and/or structure comparison.

As used herein the term “EC₅₀” refers to the half maximal effective concentration of a monoclonal or bispecific antibody or antigen-binding fragment thereof of the invention. EC₅₀ refers to the concentration of a monoclonal or bispecific antibody or antigen-binding fragment thereof for inducing a biological response (i.e., cell death) halfway between the baseline and maximum over a specified exposure time. In certain embodiments, the monoclonal antibody or antigen-binding fragment thereof or the bispecific antibody or antigen-binding fragment thereof has an EC₅₀ of less than about 1 μM, about 1000 nM to about 100 nM, about 100 nM to about 10 nM, about 10 nM to about 1 nM, about 1000 pM to about 500 pM, about 500 pM to about 200 pM, less than about 200 pM, about 200 pM to about 150 pM, about 200 pM to about 100 pM, about 100 pM to about 10 pM, or about 10 pM to about 1 pM.

According to another particular aspect, the invention relates to isolated bispecific antibodies or antigen-binding fragments thereof comprising:
a. a first heavy chain, H1;
b. a second heavy chain, H2;
c. a first light chain, L1; and
d. a second light chain, L2;
   wherein H1 and L1 form a first arm comprising a first antigen-binding domain that specifically binds a first antigen, preferably a first antigen of human origin, and wherein H2 and L2 form a second arm comprising a second antigen-binding domain that specifically binds a second antigen, preferably a second antigen of human origin, wherein
(a) H1 and H2 each comprises a CH1 region of human IgG1, IgG2, IgG3, or IgG4; and
(b) L1 and L2 each comprises a CL region of a human kappa light chain or a human lambda light chain;
   wherein H1L1 and H2L2 each comprise a charge pair selected from the group consisting of the following amino acid substitutions:
(1) G166D/E in CH1 of H1 and S114K/R in CL of L1, respectively, and G166K/R in CH1 of H2 and S114D/E in CL of L2, respectively;
(2) T187D/E in CH1 of H1 and D/N170K/R in CL of L1, respectively, and T187K/R in CH1 of H2 and D/N170D/E in CL of L2, respectively;
(3) S131D/E in CH1 of H1 and P119K/R in CL of L1, respectively, and S131K/R in CH1 of H2 and P119D/E in CL of L2, respectively;
(4) A129D/E in CH1 of H1 and S121K/R in CL of L1, respectively, and A129K/R in CH1 of H2 and S121D/E in CL of L2, respectively;
(5) G166D/E in CH1 of H2 and S114K/R in CL of L2, respectively, and G166K/R in CH1 of H1 and S114D/E in CL of L1, respectively;
(6) T187D/E in CH1 of H2 and D/N170K/R in CL of L2, respectively, and T187K/R in CH1 of H1 and D/N170D/E in CL of L1, respectively;
(7) S131D/E in CH1 of H2 and P119K/R in CL of L2, respectively, and S131K/R in CH1 of H1 and P119D/E in CL of L1, respectively; or
(8) A129D/E in CH1 of H2 and S121K/R in CL of L2, respectively, and A129K/R in CH1 of H1 and S121D/E in CL of L1, respectively.

In certain embodiments, the two heavy chains H1 and H2 each comprise a VH region, a CH1 region, and a Fc region (containing CH2 and CH3 regions), wherein the VH regions have different amino acid sequences. In certain embodiments, the two heavy chains H1 and H2 each comprise a VH region, a CH1 region, and a Fc region (containing CH2 and CH3 regions), wherein the CH1 regions have different amino acid sequences. In certain embodiments, the two heavy chains H1 and H2 each comprise a VH region, a CH1 region, and a Fc region (containing CH2 and CH3 regions), wherein the Fc regions have different amino acid sequences. In certain embodiments, the two light chains L1 and L2 each comprise a VL region and a CL region, wherein the VL regions have different amino acid sequences. In certain embodiments, the two light chains L1 and L2 each comprise a VL region and a CL region, wherein the CL regions have different amino acid sequences.

In another particular aspect, H1 and H2 form a heterodimer.

In another particular aspect, (a) a negatively charged amino acid (D or E) is introduced at G166, T187, S131, or A129 in CH1 of H1 (with a positively charged amino acid introduced at the corresponding position in CL of L1 as described above) and a positively charged amino acid (K or R) is introduced at the corresponding residue in CH1 of H2 (with a negatively charged amino acid introduced at the corresponding position in CL of L2 as described above), the VH region of H1 and the VL region of L1 have a Q39E and a Q38K substitution mutation, respectively, and the VH region of H2 and the VL region of L2 have a Q39K and a Q38E substitution mutation, respectively; or (b) a positively charged amino acid (K or R) is introduced at G166, T187, S131, or A129 in CH1 of H1 (with a negatively charged amino acid introduced at the corresponding position in CL of L1 as described above) and a negatively charged amino acid (D or E) is introduced at the corresponding residue in CH1 of H2 (with a positively charged amino acid introduced at the corresponding position in CL of L2 as described above), the VH region of H1 and the VL region of L1 have a Q39K and a Q38E substitution mutation, respectively, and the VH region of H2 and the VL region of L2 have a Q39E and a Q38K substitution mutation, respectively.

In another particular aspect, the isolated bispecific antibody or antigen-binding fragment thereof comprises the CH1 and CL regions of one of the two arms comprising amino acid substitutions at an amino acid residue corresponding to the amino acid position of SEQ ID NO:17, 18, 19, or 20 for CH1 and SEQ ID NO:21 or 22 for CL, wherein the amino acid substitutions in the CH1 and CL regions are selected from:
(1) K133C and C220X in CH1, and F209C and C214X in CL;
(2) R133C and C131X in CH1, and F209C and C214X in CL;
(3) R133C and C131X in CH1, and V209C and C214X in CL; or
(4) K133C and C220X in CH1, and V209C and C214X in CL;
   wherein X is selected from S, A or G.

In another particular aspect, the isolated bispecific antibody or antigen-binding fragment thereof comprises an anti-CD47 antibody or antigen-binding fragment arm and an anti-TAA antibody or antigen-binding fragment arm thereof, wherein CD47 and the TAA are expressed on the same cell, and is capable of specific binding to both CD47 and the TAA, preferably human CD47 and TAA. The isolated anti-CD47 antibody or antigen-binding fragment thereof can, for example, comprise the VH, CH1, VL, and CL comprising the amino acid sequences of SEQ ID: 23, 28, 24 and 29, respectively.

In another particular aspect, the isolated bispecific antibody or antigen-binding fragment comprises a first antigen-binding domain that specifically binds CD47, preferably human CD47, and a second antigen-binding domain that specifically binds CLDN18.2, preferably human CLDN18.2.

In another particular aspect, the anti-CD47 antigen-binding domain comprises the VH, CH1, VL, and CL comprising the amino acid sequences of:
(1) SEQ ID NOs: 1, 27, 2 and 29, respectively;
(2) SEQ ID NOs: 1, 28, 2 and 29, respectively;
(3) SEQ ID NOs: 1, 27, 2 and 30, respectively;
(4) SEQ ID NOs: 1, 28, 2 and 30, respectively;
(5) SEQ ID NOs: 1, 31, 2 and 33, respectively;
(6) SEQ ID NOs: 1, 32, 2 and 33, respectively;
(7) SEQ ID NOs: 1, 31, 2 and 34, respectively;
(8) SEQ ID NOs: 1, 32, 2 and 34, respectively;
(9) SEQ ID NOs: 1, 35, 2 and 37, respectively;
(10) SEQ ID NOs: 1, 36, 2 and 37, respectively;
(11) SEQ ID NOs: 1, 35, 2 and 38, respectively;
(12) SEQ ID NOs: 1, 36, 2 and 38, respectively;
(13) SEQ ID NOs: 1, 39, 2 and 41, respectively;
(14) SEQ ID NOs: 1, 40, 2 and 41, respectively;
(15) SEQ ID NOs: 1, 39, 2 and 42, respectively;
(16) SEQ ID NOs: 1, 40, 2 and 42, respectively;
(17) SEQ ID NOs: 1, 43, 2 and 45, respectively;
(18) SEQ ID NOs: 1, 44, 2 and 45, respectively;
(19) SEQ ID NOs: 1, 43, 2 and 46 respectively;
(20) SEQ ID NOs: 1, 44, 2 and 46, respectively;
(21) SEQ ID NOs: 1, 47, 2 and 49, respectively;
(22) SEQ ID NOs: 1, 48, 2 and 49, respectively;
(23) SEQ ID NOs: 1, 47, 2 and 50, respectively;
(24) SEQ ID NOs: 1, 48, 2 and 50, respectively;
(25) SEQ ID NOs: 1, 51, 2 and 53, respectively;
(26) SEQ ID NOs: 1, 52, 2 and 53, respectively;
(27) SEQ ID NOs: 1, 51, 2 and 54, respectively;
(28) SEQ ID NOs: 1, 52, 2 and 54, respectively;
(29) SEQ ID NOs: 1, 55, 2 and 57, respectively;
(30) SEQ ID NOs: 1, 56, 2 and 57, respectively;
(31) SEQ ID NOs: 1, 55, 2 and 58, respectively;
(32) SEQ ID NOs: 1, 56, 2 and 58, respectively;
(33) SEQ ID NOs: 23, 27, 24 and 29, respectively;
(34) SEQ ID NOs: 23, 28, 24 and 29, respectively;
(35) SEQ ID NOs: 23, 27, 24 and 30, respectively;
(36) SEQ ID NOs: 23, 28, 24 and 30, respectively;
(37) SEQ ID NOs: 25, 31, 26 and 33, respectively;
(38) SEQ ID NOs: 25, 32, 26 and 33, respectively;
(39) SEQ ID NOs: 25, 31, 26 and 34, respectively;
(40) SEQ ID NOs: 25, 32, 26 and 34, respectively;
(41) SEQ ID NOs: 23, 35, 24 and 37, respectively;
(42) SEQ ID NOs: 23, 36, 24 and 37, respectively;
(43) SEQ ID NOs: 23, 35, 24 and 38, respectively;
(44) SEQ ID NOs: 23, 36, 24 and 38, respectively;
(45) SEQ ID NOs: 25, 39, 26 and 41, respectively;
(46) SEQ ID NOs: 25, 40, 26 and 41, respectively;
(47) SEQ ID NOs: 25, 39, 26 and 42, respectively;
(48) SEQ ID NOs: 25, 40, 26 and 42, respectively;
(49) SEQ ID NOs: 23, 43, 24 and 45, respectively;
(50) SEQ ID NOs: 23, 44, 24 and 45, respectively;
(51) SEQ ID NOs: 23, 43, 24 and 46 respectively;
(52) SEQ ID NOs: 23, 44, 24 and 46, respectively;
(53) SEQ ID NOs: 25, 47, 26 and 49, respectively;
(54) SEQ ID NOs: 25, 48, 26 and 49, respectively;
(55) SEQ ID NOs: 25, 47, 26 and 50, respectively;
(56) SEQ ID NOs: 25, 48, 26 and 50, respectively;
(57) SEQ ID NOs: 23, 51, 24 and 53, respectively;
(58) SEQ ID NOs: 23, 52, 24 and 53, respectively;
(59) SEQ ID NOs: 23, 51, 24 and 54, respectively;
(60) SEQ ID NOs: 23, 52, 24 and 54, respectively;
(61) SEQ ID NOs: 25, 55, 26 and 57, respectively;
(62) SEQ ID NOs: 25, 56, 26 and 57, respectively;
(63) SEQ ID NOs: 25, 55, 26 and 58, respectively; or
(64) SEQ ID NOs: 25, 56, 26 and 58, respectively.

In another particular aspect, the isolated bispecific antibody or antigen-binding fragment is an anti-CD47/anti-CLDN18.2 bispecific antibody, wherein the first antigen-binding domain comprises the VH, CH1, VL, and CL, the second antigen-binding domain comprises the VH, CH1, VL, and CL, comprising the amino acid sequences of:
(1) SEQ ID: 1, 28, 2, 29, 3, 63, 4 and 64, respectively;
(2) SEQ ID: 1, 36, 2, 37, 3, 67, 4 and 68, respectively;
(3) SEQ ID: 1, 44, 2, 45, 3, 71, 4 and 72, respectively;
(4) SEQ ID: 1, 52, 2, 53, 3, 73, 4 and 74, respectively;
(5) SEQ ID: 1, 31, 2, 34, 3, 65, 4 and 66, respectively;
(6) SEQ ID: 1, 39, 2, 42, 3, 69, 4 and 70, respectively;
(7) SEQ ID: 1, 47, 2, 50, 3, 75, 4 and 76, respectively;
(8) SEQ ID: 1, 55, 2, 58, 3, 77, 4 and 78, respectively;
(9) SEQ ID: 23, 28, 24, 29, 59, 63, 60 and 64, respectively;
(10) SEQ ID: 23, 36, 24, 37, 59, 67, 60 and 68, respectively;
(11) SEQ ID: 23, 44, 24, 45, 59, 71, 60 and 72, respectively;
(12) SEQ ID: 23, 52, 24, 53, 59, 73, 60 and 74, respectively;
(13) SEQ ID: 25, 31, 26, 34, 61, 65, 62 and 66, respectively;
(14) SEQ ID: 25, 39, 26, 42, 61, 69, 62 and 70, respectively;
(15) SEQ ID: 25, 47, 26, 50, 61, 75, 62 and 76, respectively; or
(16) SEQ ID: 25, 55, 26, 58, 61, 77, 62 and 78, respectively.

In another particular aspect, the isolated bispecific antibody or antigen-binding fragment thereof comprises an anti-immune cell modulator (ICM) antibody or antigen-binding fragment arm thereof and is capable of specific binding to ICM, preferably a human ICM. The ICM can, for example, be selected from the group consisting of CD3, CD16, CD27, CD28, CD40, CD122, NKp46, OX40, 4-1BB, GITR, ICOS, CTLA-4, PD-1, LAG-3, TIM-3, VISTA, SIGLEC7, SIGLEC9, KIR, BTLA, B7-H3, and other cell surface immune regulatory molecules.

In another particular aspect, the anti-ICM antibody or antigen-binding fragment thereof is an anti-CD3 antibody or antigen-binding fragment thereof and is capable of specific binding to CD3, preferably human CD3. The isolated anti-CD3 antibody or antigen-binding fragment thereof can, for example, comprise the VH, CH1, VL, and CL comprising the amino acid sequences of SEQ ID: 9, 10, 11 and 12, respectively.

In another particular aspect, the isolated bispecific antibody or antigen-binding fragment thereof is an anti-CD3/anti-DLL3 bispecific antibody or antigen-binding fragment thereof. In certain embodiments, the anti-CD3/anti-DLL3 bispecific antibody or antigen-binding fragment thereof is capable of engaging DLL3-expressing cancer cells with T cells and activate T cell-mediated killing of cancer cells. In another particular aspect, the first antigen-binding domain comprises the VH, CH1, VL, and CL comprising the amino acid sequences of SEQ ID: 9, 10, 11 and 12, respectively, and the second antigen-binding domain comprises the VH, CH1, VL, and CL comprising the amino acid sequences of SEQ ID:13, 14, 15 and 16, respectively.

Full length bispecific antibodies of the invention can be generated for example using Fab arm exchange (or half molecule exchange) between two mono specific bivalent antibodies by introducing substitutions at the heavy chain CH3 interface in each half molecule to favor heterodimer formation of two antibody half molecules having distinct specificity either in vitro in cell-free environment or using co-expression. The Fab arm exchange reaction is the result of a disulfide-bond isomerization reaction and dissociation-association of CH3 domains. The heavy-chain disulfide bonds in the hinge regions of the parent mono specific antibodies are reduced. The resulting free cysteines of one of the parent monospecific antibodies form an inter heavy-chain disulfide bond with cysteine residues of a second parent monospecific antibody molecule and simultaneously CH3 domains of the parent antibodies release and reform by dissociation-association. The CH3 domains of the Fab arms can be engineered to favor heterodimerization over homodimerization. The resulting product is a bispecific antibody having two Fab arms or half molecules which each bind a distinct epitope, i.e., an epitope on CD3, an epitope on DLL3, and combinations thereof, or an epitope on CD47, an epitope on CLDN18.2, and combinations thereof

“Homodimerization” as used herein refers to an interaction of two heavy chains having identical CH3 amino acid sequences. “Homodimer” as used herein refers to an antibody having two heavy chains with identical CH3 amino acid sequences.

“Heterodimerization” as used herein refers to an interaction of two heavy chains having non-identical CH3 amino acid sequences. “Heterodimer” as used herein refers to an antibody having two heavy chains with non-identical CH3 amino acid sequences.

The “knob-in-hole” strategy (see, e.g., PCT Publ. No. WO2006/028936) can be used to generate full length bispecific antibodies. Briefly, selected amino acids forming the interface of the CH3 domains in human IgG can be mutated at positions affecting CH3 domain interactions to promote heterodimer formation. An amino acid with a small side chain (hole) is introduced into a heavy chain of an antibody specifically binding a first antigen and an amino acid with a large side chain (knob) is introduced into a heavy chain of an antibody specifically binding a second antigen. After co-expression of the two antibodies, a heterodimer is formed as a result of the preferential interaction of the heavy chain with a “hole” with the heavy chain with a “knob.” Exemplary CH3 substitution pairs forming a knob and a hole are (expressed as modified positions in the first CH3 domain of the first heavy chain/modified position in the second CH3 domain of the second heavy chain): T366Y/F405A, T366W/ F405W, F405W/Y407A, T394W/Y407T, T394S/Y407A, T366W/T394S, F405W/T394S and T366W/T366S_L368A_Y407V.

Other strategies such as promoting heavy chain heterodimerization using electrostatic interactions by substituting positively charged residues at one CH3 surface and negatively charged residues at a second CH3 surface can be used, as described in U.S. Pat. Publ. No. 2010/0015133; U.S. Pat. Publ. No. 2009/0182127; U.S. Pat. Publ. No. 2010/028637; or U.S. Pat. Publ. No. 2011/0123532. In other strategies, heterodimerization can be promoted by the following substitutions (expressed as modified position in the first CH3 domain of the first heavy chain/modified position in the second CH3 domain of the second heavy chain): L351Y_F405AY407V/T394W, T366I_K392M_T394W/F405A_Y407V, T366L_K392M_T394W/F405A_Y407V, L351Y_Y407A/T366A_K409F, L351Y_Y407A/T366V K409F Y407A/T366A_K409F, or T350V_L351Y_F405A Y407V/T350V_T366L_K392L_T394W as described in U.S. Pat. Publ. No. 2012/0149876 or U.S. Pat. Publ. No. 2013/0195849.

In addition to methods described above, bispecific antibodies of the invention can be generated in vitro in a cell-free environment by introducing asymmetrical mutations in the CH3 regions of two mono specific homodimeric antibodies and forming the bispecific heterodimeric antibody from two parent monospecific homodimeric antibodies in reducing conditions to allow disulfide bond isomerization according to methods described in PCT Pat. Publ. No. WO2011/131746. In the methods, the first monospecific bivalent antibody and the second monospecific bivalent antibody are engineered to have certain substitutions at the CH3 domain that promotes heterodimer stability; the antibodies are incubated together under reducing conditions sufficient to allow the cysteines in the hinge region to undergo disulfide bond isomerization; thereby generating the bispecific antibody by Fab arm exchange. The incubation conditions can optionally be restored to non-reducing conditions. Exemplary reducing agents that can be used are 2-mercaptoethylamine (2-MEA), dithiothreitol (DTT), dithioerythritol (DTE), glutathione, tris (2-carboxyethyl) phosphine (TCEP), L-cysteine and beta-mercaptoethanol, preferably a reducing agent selected from the group consisting of: 2-mercaptoethylamine, dithiothreitol and tris (2-carboxyethyl) phosphine. For example, incubation for at least 90 minutes at a temperature of at least 20° C. in the presence of at least 25 mM 2-MEA or in the presence of at least 0.5 mM dithiothreitol at a pH from 5-8, for example at pH of 7.0 or at pH of 7.4 can be used.

Full length bispecific antibodies of the invention can be generated using a combination of the heterodimerization approaches above and several approaches as follows: (a) shifting the HC/LC interchain disulfide bond on one arm of the bispecific antibody (see, e.g., WO2021/126538, which is incorporated by reference herein in its entirety); (b) introducing charge pairs to the VH/VL interface; (c) introducing charge pairs to the CH1/CL interface; or (d) a combination of some or all the approaches described in (a)-(c).

In another general aspect, the invention relates to an isolated nucleic acid encoding a bispecific antibody or antigen-binding fragment thereof of the invention. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding bispecific antibodies or antigen-binding fragments thereof of the invention can be altered without changing the amino acid sequences of the proteins.

In another general aspect, the invention relates to a vector comprising an isolated nucleic acid encoding a bispecific antibody or antigen-binding fragment thereof of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments of the invention. Such techniques are well known to those skilled in the art in view of the present disclosure.

In another general aspect, the invention relates to a host cell comprising a vector comprising an isolated nucleic acid encoding a bispecific antibody or antigen-binding fragment thereof of the invention. Any host cell known to those skilled in the art in view of the present disclosure can be used for recombinant expression of antibodies or antigen-binding fragments thereof of the invention. In some embodiments, the host cells are E. coli TG1 or BL21 cells (for expression of, e.g., a scFv or Fab antibody), CHO-DG44 or CHO-Kl cells or HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another general aspect, the invention relates to a method of producing a bispecific antibody or antigen-binding fragment thereof of the invention, comprising culturing a cell comprising a nucleic acid encoding the bispecific antibody or antigen-binding fragment thereof under conditions to produce a bispecific antibody or antigen-binding fragment thereof of the invention, and recovering the bispecific antibody or antigen-binding fragment thereof from the cell or cell culture (e.g., from the supernatant). Expressed bispecific antibodies or antigen-binding fragments thereof can be harvested from the cells and purified according to conventional techniques known in the art and as described herein.

### Pharmaceutical Compositions

In another general aspect, the invention relates to a pharmaceutical composition, comprising an isolated bispecific antibody or antigen-binding fragment thereof of the invention and a pharmaceutically acceptable carrier. The term “pharmaceutical composition” as used herein means a product comprising an antibody of the invention together with a pharmaceutically acceptable carrier. Antibodies of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in an antibody pharmaceutical composition can be used in the invention.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include buffers, diluents, solvents, tonicity regulating agents, preservatives, stabilizers, and chelating agents. One or more pharmaceutically acceptable carriers can be used in formulating the pharmaceutical compositions of the invention.

In one embodiment of the invention, the pharmaceutical composition is a liquid formulation. A preferred example of a liquid formulation is an aqueous formulation, i.e., a formulation comprising water. The liquid formulation can comprise a solution, a suspension, an emulsion, a microemulsion, a gel, and the like. An aqueous formulation typically comprises at least 50% w/w water, or at least 60%, 70%, 75%, 80%, 85%, 90%, or at least 95% w/w of water.

In one embodiment, the pharmaceutical composition can be formulated as an injectable which can be injected, for example, via an injection device (e.g., a syringe or an infusion pump). The injection can be delivered subcutaneously, intramuscularly, intraperitoneally, intravitreally, or intravenously, for example.

In another embodiment, the pharmaceutical composition is a solid formulation, e.g., a freeze-dried or spray-dried composition, which can be used as is, or whereto the physician or the patient adds solvents, and/or diluents prior to use. Solid dosage forms can include tablets, such as compressed tablets, and/or coated tablets, and capsules (e.g., hard or soft gelatin capsules). The pharmaceutical composition can also be in the form of sachets, dragees, powders, granules, lozenges, or powders for reconstitution, for example.

The dosage forms can be immediate release, in which case they can comprise a water-soluble or dispersible carrier, or they can be delayed release, sustained release, or modified release, in which case they can comprise water-insoluble polymers that regulate the rate of dissolution of the dosage form in the gastrointestinal tract or under the skin.

In other embodiments, the pharmaceutical composition can be delivered intranasally, intrabuccally, or sublingually.

The pH in an aqueous formulation can be between pH 3 and pH 10. In one embodiment of the invention, the pH of the formulation is from about 7.0 to about 9.5. In another embodiment of the invention, the pH of the formulation is from about 3.0 to about 7.0.

In another embodiment of the invention, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer can be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments of the invention.

In another embodiment of the invention, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative can be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments of the invention.

In another embodiment of the invention, the pharmaceutical composition comprises an isotonic agent. Non-limiting examples of the isotonic agents include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethylene glycol (e.g. PEG400), and mixtures thereof Another example of an isotonic agent includes a sugar. Non-limiting examples of sugars may be mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethylcellulose. Another example of an isotonic agent is a sugar alcohol, wherein the term “sugar alcohol” is defined as a C(4-8) hydrocarbon having at least one —OH group. Non-limiting examples of sugar alcohols include mannitol, sorbitol, inositol, galactitol, dulcitol, xylitol, and arabitol. Pharmaceutical compositions comprising each isotonic agent listed in this paragraph constitute alternative embodiments of the invention. The isotonic agent can be present individually or in the aggregate, in a concentration from about 0.01 mg/ml to about 50 mg/ml, for example from about 0.1 mg/ml to about 20 mg/ml. Pharmaceutical compositions comprising each one of these specific isotonic agents constitute alternative embodiments of the invention.

In another embodiment of the invention, the pharmaceutical composition comprises a chelating agent. Non-limiting examples of chelating agents include citric acid, aspartic acid, salts of ethylenediaminetetraacetic acid (EDTA), and mixtures thereof The chelating agent can be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments of the invention.

In another embodiment of the invention, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment of the invention, the pharmaceutical composition comprises a stabilizer, wherein said stabilizer is carboxy-/hydroxycellulose and derivates thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulfur-containing substances such as monothioglycerol), or thioglycolic acid. The stabilizer can be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments of the invention.

In further embodiments of the invention, the pharmaceutical composition comprises one or more surfactants, preferably a surfactant, at least one surfactant, or two different surfactants. The term “surfactant” refers to any molecules or ions that are comprised of a water-soluble (hydrophilic) part, and a fat-soluble (lipophilic) part. The surfactant can, for example, be selected from the group consisting of anionic surfactants, cationic surfactants, nonionic surfactants, and/or zwitterionic surfactants. The surfactant can be present individually or in the aggregate, in a concentration from about 0.1 mg/ml to about 20 mg/ml. Pharmaceutical compositions comprising each one of these specific surfactants constitute alternative embodiments of the invention.

In a further embodiment of the invention, the pharmaceutical composition comprises one or more protease inhibitors, such as, e.g., EDTA, and/or benzamidine hydrochloric acid (HC1). The protease inhibitor can be present individually or in the aggregate, in a concentration from about 0.1 mg/ml to about 20 mg/ml. Pharmaceutical compositions comprising each one of these specific protease inhibitors constitute alternative embodiments of the invention.

In another general aspect, the invention relates to a method of producing a pharmaceutical composition comprising a bispecific antibody or antigen-binding fragment thereof of the invention, comprising combining a bispecific antibody or antigen-binding fragment thereof with a pharmaceutically acceptable carrier to obtain the pharmaceutical composition.

### Methods of Use

In a general aspect, the invention relates to a method of targeting CD47 and a TAA (such as CLDN18.2) that are both expressed on a cancer cell surface in a subject in need thereof by activating macrophage-mediated phagocytosis of the cancer cell, comprising administering to the subject in need thereof an anti-TAA/anti-CD47 bispecific antibody or antigen-binding fragment thereof or a pharmaceutical composition of the invention.

In a general aspect, the invention relates to a method of targeting DLL3 that is expressed on a cancer cell surface in a subject in need thereof by engaging T cells; the method comprises administering to the subject in need thereof an anti-CD3/anti-DLL3 bispecific antibody or antigen-binding fragment thereof or a pharmaceutical composition of the invention.

The functional activity of monoclonal antibodies or antigen-binding fragments thereof that bind a target antigen (e.g., an ICM, such as CD3), or bispecific antibodies and antigen-binding fragments thereof that bind both a TAA (e.g., DLL3) and a T cell target antigen (e.g., an ICM, such as CD3) or bind both a TAA and CD47 on the same cell can be characterized by methods known in the art and as described herein. Methods for characterizing bispecific antibodies and antigen-binding fragments thereof that bind both a TAA (e.g., DLL3) and a T cell target antigen (e.g., CD3) include, but are not limited to, cell-engaging based cross-linking assay or T cell activation assay, and affinity and specificity assays including Biacore, ELISA, FACS and OctetRed analysis. Methods for characterizing bispecific antibodies and antigen-binding fragments thereof that bind both a TAA (e.g., CLDN18.2) and CD47 on the same cell include, but are not limited to, affinity and specificity assays including Biacore, ELISA, FACS and OctetRed analysis. According to particular embodiments, the methods for characterizing bispecific antibodies and antigen-binding fragments thereof that bind both DLL3 and CD3 include those described below. The functional activity of monoclonal antibodies or antigen-binding fragments thereof that bind an ICM, or bispecific antibodies and antigen-binding fragments thereof that bind both a TAA (e.g., DLL3) and an ICM other than CD3 can be characterized by methods similar to those above.

In another general aspect, the invention relates to a method of targeting one or two antigens expressed on a cancer cell surface in a subject in need thereof including using CD3-mediated T cell activation, and/or treating cancer in a subject in need thereof, comprising administering to the subject a pharmaceutical composition comprising the isolated bispecific antibody or antigen-binding fragment thereof of and a pharmaceutically acceptable carrier. optionally the cancer is selected from the group consisting of a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

In another general aspect, the invention relates to a method of producing the bispecific antibody or antigen-binding fragment thereof, comprising culturing a cell comprising a nucleic acid encoding the bispecific antibody or antigen-binding fragment thereof under conditions to produce the bispecific antibody or antigen-binding fragment thereof, and recovering the bispecific antibody or antigen-binding fragment thereof from the cell or culture.

In another general aspect, the invention relates to a method of producing a pharmaceutical composition comprising the bispecific antibody or antigen-binding fragment thereof, comprising combining the bispecific antibody or antigen-binding fragment thereof with a pharmaceutically acceptable carrier to obtain the pharmaceutical composition.

In another general aspect, the invention relates to a method of treating a cancer in a subject in need thereof, comprising administering to the subject in need thereof an isolated humanized bispecific antibody or antigen-binding fragment thereof such as anti-CD47/anti-CLDN18.2 bispecific antibody, or anti-CD3/anti-DLL3 bispecific antibody or a pharmaceutical composition of the invention. The cancer can be any liquid or solid cancer, for example, it can be selected from, but not limited to, a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

According to embodiments of the invention, the pharmaceutical composition comprises a therapeutically effective amount of an anti-CD47/anti-CLDN18.2 or an anti-CD3/anti-DLL3 bispecific antibody or antigen-binding fragment thereof of the invention. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to anti-CD47/anti-CLDN18.2 or anti-CD3/anti-DLL3 bispecific antibodies or antigen-binding fragments thereof, a therapeutically effective amount means an amount of the anti-CD47/anti-CLDN18.2 or anti-CD3/anti-DLL3 bispecific antibody or antigen-binding fragment thereof that modulates an immune response in a subject in need thereof. Also as used herein with reference to anti-CD47/anti-CLDN18.2 or anti-CD3/anti-DLL3 bispecific antibodies or antigen-binding fragments thereof, a therapeutically effective amount means an amount of the anti-CD47/anti-CLDN18.2 or anti-CD3/anti-DLL3 bispecific antibody or antigen-binding fragment thereof that results in treatment of a disease, disorder, or condition; prevents or slows the progression of the disease, disorder, or condition; or reduces or completely alleviates symptoms associated with the disease, disorder, or condition.

According to particular embodiments, the disease, disorder or condition to be treated is cancer, preferably a cancer selected from the group consisting of a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors. According to other particular embodiments, the disease, disorder or condition to be treated is an inflammatory disease, a metabolic disease, or any other disease where a bispecific antibody can be used as a therapy.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subj ect.

According to particular embodiments, provided is a composition used in the treatment of a cancer. For cancer therapy, the composition can be used in combination with another treatment including, but not limited to, a chemotherapy, an anti-TIM-3 mAb, an anti-LAG-3 mAb, an anti-CD73 mAb, an anti-apelin mAb, an anti-CTLA-4 antibody, an anti-EGFR mAb, an anti-HER-2 mAb, an anti-CD19 mAb, an anti-CD20 mAb, an anti-CD33 mAb, an anti-TIP-1 mAb, an anti-CLDN18.2 mAb, an anti-PD-L1 antibody, an anti-PD-1 antibody, a PD-1/PD-L1 therapy, other immuno-oncology drugs, an antiangiogenic agent, a radiation therapy, an antibody-drug conjugate (ADC), a targeted therapy, or other anticancer drugs.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

### EMBODIMENTS

The invention provides also the following non-limiting embodiments.

Embodiment 1 is an isolated bispecific antibody or antigen-binding fragment thereof comprising:
a. a first heavy chain, H1;
b. a second heavy chain, H2;
c. a first light chain, L1; and
d. a second light chain, L2;
   wherein H1 and L1 form a first arm comprising a first antigen-binding domain that specifically binds a first antigen, preferably a first antigen of human origin, and
   wherein H2 and L2 form a second arm comprising a second antigen-binding domain that specifically binds a second antigen, preferably a second antigen of human origin, wherein
(a) H1 and H2 each comprises a CH1 region of human IgG1, IgG2, IgG3, or IgG4; and
(b) L1 and L2 each comprises a CL region of a human kappa light chain or a human lambda light chain;
   wherein H1L1 and H2L2 each comprise a charge pair selected from the group consisting of the following amino acid substitutions:
(1) G166D/E in CH1 of H1 and S114K/R in CL of L1, respectively, and G166K/R in CH1 of H2 and S114D/E in CL of L2, respectively;
(2) T187D/E in CH1 of H1 and D/N170K/R in CL of L1, respectively, and T187K/R in CH1 of H2 and D/N170D/E in CL of L2, respectively;
(3) S131D/E in CH1 of H1 and P119K/R in CL of L1, respectively, and S131K/R in CH1 of H2 and P119D/E in CL of L2, respectively;
(4) A129D/E in CH1 of H1 and S121K/R in CL of L1, respectively, and A129K/R in CH1 of H2 and S121D/E in CL of L2, respectively;
(5) G166D/E in CH1 of H2 and S114K/R in CL of L2, respectively, and G166K/R in CH1 of H1 and S114D/E in CL of L1, respectively;
(6) T187D/E in CH1 of H2 and D/N170K/R in CL of L2, respectively, and T187K/R in CH1 of H1 and D/N170D/E in CL of L1, respectively;
(7) S131D/E in CH1 of H2 and P119K/R in CL of L2, respectively, and S131K/R in CH1 of H1 and P119D/E in CL of L1, respectively; or
(8) A129D/E in CH1 of H2 and S121K/R in CL of L2, respectively, and A129K/R in CH1 of H1 and S121D/E in CL of L1, respectively.

Embodiment 2 is the isolated bispecific antibody or antigen-binding fragment thereof of embodiment 1, wherein
(a) the two heavy chains H1 and H2 each comprise a VH region, a CH1 region, and a Fc region (containing CH2 and CH3 regions), wherein the VH regions have different amino acid sequences;
(b) the two heavy chains H1 and H2 each comprise a VH region, a CH1 region, and a Fc region (containing CH2 and CH3 regions), wherein the CH1 regions have different amino acid sequences;
(c) the two heavy chains H1 and H2 each comprise a VH region, a CH1 region, and a Fc region (containing CH2 and CH3 regions), wherein the Fc regions have different amino acid sequences;
(d) the two light chains L1 and L2 each comprise a VL region and a CL region, wherein the VL regions have different amino acid sequences; and/or
(e) the two light chains L1 and L2 each comprise a VL region and a CL region, wherein the CL regions have different amino acid sequences.

Embodiment 3 is the isolated bispecific antibody or antigen-binding fragment thereof of embodiment 2, wherein H1 and H2 form a heterodimer.

Embodiment 4 is the isolated bispecific antibody or antigen-binding fragment thereof of any one of embodiments 1 to 3, wherein the isolated antibody or antigen-binding fragment comprises:
(a) a negatively charged amino acid (D or E) at G166, T187, S131, or A129 in CH1 of H1, the VH region of H1 and the VL region of L1 have a Q39E and a Q38K substitution mutation, respectively, and the VH region of H2 and the VL region of L2 have a Q39K and a Q38E substitution mutation, respectively; or
(b) a positively charged amino acid (K or R) at G166, T187, S131, or A129 in CH1 of H1, the VH region of H1 and the VL region of L1 have a Q39K and a Q38E substitution mutation, respectively, and the VH region of H2 and the VL region of L2 have a Q39E and a Q38K substitution mutation, respectively.

Embodiment 5 is the isolated bispecific antibody or antigen-binding fragment thereof of any one of embodiments 1 to 4, wherein the CH1 and CL regions of one of the two arms comprise amino acid substitutions at an amino acid residue corresponding to the amino acid position of SEQ ID NO:17, 18, 19, or 20 for CH1 and SEQ ID NO:21 or 22 for CL;

wherein the amino acid substitutions in the CH1 and CL regions are selected from:
(1) K133C and C220X in CH1, and F209C and C214X in CL;
(2) R133C and C131X in CH1, and F209C and C214X in CL;
(3) R133C and C131X in CH1, and V209C and C214X in CL; or
(4) K133C and C220X in CH1, and V209C and C214X in CL;
   wherein X is selected from S, A or G.

Embodiment 6 is the isolated bispecific antibody or antigen-binding fragment thereof of any one of embodiments 1 to 5, wherein the first antigen-binding domain specifically binds CD47, preferably human CD47, and the second antigen-binding domain specifically binds a TAA expressed on the same cell as CD47, preferably human TAA.

Embodiment 7 is the isolated bispecific antibody or antigen-binding fragment thereof of embodiment 6, wherein the second antigen-binding domain specifically binds CLDN18.2 expressed on the same cell as CD47, preferably human CLDN18.2.

Embodiment 8 is the isolated bispecific antibody or antigen-binding fragment thereof of embodiment 6 or 7, wherein the anti-CD47 antigen-binding domain comprises the VH, CH1, VL, and CL comprising the amino acid sequences of:
(1) SEQ ID NOs: 1, 27, 2 and 29, respectively;
(2) SEQ ID NOs: 1, 28, 2 and 29, respectively;
(3) SEQ ID NOs: 1, 27, 2 and 30, respectively;
(4) SEQ ID NOs: 1, 28, 2 and 30, respectively;
(5) SEQ ID NOs: 1, 31, 2 and 33, respectively;
(6) SEQ ID NOs: 1, 32, 2 and 33, respectively;
(7) SEQ ID NOs: 1, 31, 2 and 34, respectively;
(8) SEQ ID NOs: 1, 32, 2 and 34, respectively;
(9) SEQ ID NOs: 1, 35, 2 and 37, respectively;
(10) SEQ ID NOs: 1, 36, 2 and 37, respectively;
(11) SEQ ID NOs: 1, 35, 2 and 38, respectively;
(12) SEQ ID NOs: 1, 36, 2 and 38, respectively;
(13) SEQ ID NOs: 1, 39, 2 and 41, respectively;
(14) SEQ ID NOs: 1, 40, 2 and 41, respectively;
(15) SEQ ID NOs: 1, 39, 2 and 42, respectively;
(16) SEQ ID NOs: 1, 40, 2 and 42, respectively;
(17) SEQ ID NOs: 1, 43, 2 and 45, respectively;
(18) SEQ ID NOs: 1, 44, 2 and 45, respectively;
(19) SEQ ID NOs: 1, 43, 2 and 46 respectively;
(20) SEQ ID NOs: 1, 44, 2 and 46, respectively;
(21) SEQ ID NOs: 1, 47, 2 and 49, respectively;
(22) SEQ ID NOs: 1, 48, 2 and 49, respectively;
(23) SEQ ID NOs: 1, 47, 2 and 50, respectively;
(24) SEQ ID NOs: 1, 48, 2 and 50, respectively;
(25) SEQ ID NOs: 1, 51, 2 and 53, respectively;
(26) SEQ ID NOs: 1, 52, 2 and 53, respectively;
(27) SEQ ID NOs: 1, 51, 2 and 54, respectively;
(28) SEQ ID NOs: 1, 52, 2 and 54, respectively;
(29) SEQ ID NOs: 1, 55, 2 and 57, respectively;
(30) SEQ ID NOs: 1, 56, 2 and 57, respectively;
(31) SEQ ID NOs: 1, 55, 2 and 58, respectively;
(32) SEQ ID NOs: 1, 56, 2 and 58, respectively;
(33) SEQ ID NOs: 23, 27, 24 and 29, respectively;
(34) SEQ ID NOs: 23, 28, 24 and 29, respectively;
(35) SEQ ID NOs: 23, 27, 24 and 30, respectively;
(36) SEQ ID NOs: 23, 28, 24 and 30, respectively;
(37) SEQ ID NOs: 25, 31, 26 and 33, respectively;
(38) SEQ ID NOs: 25, 32, 26 and 33, respectively;
(39) SEQ ID NOs: 25, 31, 26 and 34, respectively;
(40) SEQ ID NOs: 25, 32, 26 and 34, respectively;
(41) SEQ ID NOs: 23, 35, 24 and 37, respectively;
(42) SEQ ID NOs: 23, 36, 24 and 37, respectively;
(43) SEQ ID NOs: 23, 35, 24 and 38, respectively;
(44) SEQ ID NOs: 23, 36, 24 and 38, respectively;
(45) SEQ ID NOs: 25, 39, 26 and 41, respectively;
(46) SEQ ID NOs: 25, 40, 26 and 41, respectively;
(47) SEQ ID NOs: 25, 39, 26 and 42, respectively;
(48) SEQ ID NOs: 25, 40, 26 and 42, respectively;
(49) SEQ ID NOs: 23, 43, 24 and 45, respectively;
(50) SEQ ID NOs: 23, 44, 24 and 45, respectively;
(51) SEQ ID NOs: 23, 43, 24 and 46 respectively;
(52) SEQ ID NOs: 23, 44, 24 and 46, respectively;
(53) SEQ ID NOs: 25, 47, 26 and 49, respectively;
(54) SEQ ID NOs: 25, 48, 26 and 49, respectively;
(55) SEQ ID NOs: 25, 47, 26 and 50, respectively;
(56) SEQ ID NOs: 25, 48, 26 and 50, respectively;
(57) SEQ ID NOs: 23, 51, 24 and 53, respectively;
(58) SEQ ID NOs: 23, 52, 24 and 53, respectively;
(59) SEQ ID NOs: 23, 51, 24 and 54, respectively;
(60) SEQ ID NOs: 23, 52, 24 and 54, respectively;
(61) SEQ ID NOs: 25, 55, 26 and 57, respectively;
(62) SEQ ID NOs: 25, 56, 26 and 57, respectively;
(63) SEQ ID NOs: 25, 55, 26 and 58, respectively; or
(64) SEQ ID NOs: 25, 56, 26 and 58, respectively.

Embodiment 9 is the isolated bispecific antibody or antigen-binding fragment thereof of embodiment 8, wherein the first antigen-binding domain comprises the VH, CH1, VL, and CL, and the second antigen-binding domain comprises the VH, CH1, VL, and CL, comprising the amino acid sequences of:
(1) SEQ ID: 1, 28, 2, 29, 3, 63, 4 and 64, respectively;
(2) SEQ ID: 1, 36, 2, 37, 3, 67, 4 and 68, respectively;
(3) SEQ ID: 1, 44, 2, 45, 3, 71, 4 and 72, respectively;
(4) SEQ ID: 1, 52, 2, 53, 3, 73, 4 and 74, respectively;
(5) SEQ ID: 1, 31, 2, 34, 3, 65, 4 and 66, respectively;
(6) SEQ ID: 1, 39, 2, 42, 3, 69, 4 and 70, respectively;
(7) SEQ ID: 1, 47, 2, 50, 3, 75, 4 and 76, respectively;
(8) SEQ ID: 1, 55, 2, 58, 3, 77, 4 and 78, respectively;
(9) SEQ ID: 23, 28, 24, 29, 59, 63, 60 and 64, respectively;
(10) SEQ ID: 23, 36, 24, 37, 59, 67, 60 and 68, respectively;
(11) SEQ ID: 23, 44, 24, 45, 59, 71, 60 and 72, respectively;
(12) SEQ ID: 23, 52, 24, 53, 59, 73, 60 and 74, respectively;
(13) SEQ ID: 25, 31, 26, 34, 61, 65, 62 and 66, respectively;
(14) SEQ ID: 25, 39, 26, 42, 61, 69, 62 and 70, respectively;
(15) SEQ ID: 25, 47, 26, 50, 61, 75, 62 and 76, respectively; or
(16) SEQ ID: 25, 55, 26, 58, 61, 77, 62 and 78, respectively.

Embodiment 10 is the isolated bispecific antibody or antigen-binding fragment thereof of any one of embodiments 1 to 5, wherein the isolated bispecific antibody or antigen-binding fragment thereof comprises an anti-immune cell modulator (ICM) antibody or antigen-binding fragment arm thereof and is capable of specific binding to ICM.

Embodiment 11 is the isolated bispecific antibody or antigen-binding fragment thereof of embodiment 10, wherein the ICM is selected from the group consisting of CD3, CD16, CD27, CD28, CD40, CD122, NKp46, OX40, 4-1BB, GITR, ICOS, CTLA-4, PD-1, LAG-3, TIM-3, VISTA, SIGLEC7, SIGLEC9, KIR, BTLA, B7-H3, and other cell surface immune regulatory molecules.

Embodiment 12 is the isolated bispecific antibody or antigen-binding fragment thereof of any one of embodiments 1 to 5 and 10 to 11, wherein the first antigen-binding domain specifically binds CD3, preferably human CD3, and comprises the VH, CH1, VL, and CL comprising the amino acid sequences of SEQ ID: 9, 10, 11 and 12, respectively.

Embodiment 13 is the isolated bispecific antibody or antigen-binding fragment thereof of any one of embodiments 1 to 5 and 10 to 12, wherein the first antigen-binding domain specifically binds CD3, preferably human CD3, and the second antigen-binding domain specifically binds DLL3, preferably human DLL3.

Embodiment 14 is the isolated bispecific antibody or antigen-binding fragment thereof of embodiment 13, wherein the first antigen-binding domain comprises the VH, CH1, VL, and CL comprising the amino acid sequences of SEQ ID:9, 10, 11 and 12, respectively, and the second antigen-binding domain comprises the VH, CH1, VL, and CL comprising the amino acid sequences of SEQ ID:13, 14, 15 and 16.

Embodiment 15 is an isolated nucleic acid encoding the bispecific antibody or antigen-binding fragment of any one of embodiments 1 to 14.

Embodiment 16 is a vector comprising the isolated nucleic acid of embodiment 15.

Embodiment 17 is a host cell comprising the vector of embodiment 16.

Embodiment 18 is a pharmaceutical composition, comprising the isolated bispecific antibody or antigen-binding fragment thereof of any one of embodiments 1 to 14 and a pharmaceutically acceptable carrier.

Embodiment 19 is a method of targeting DLL3 that is expressed on a cancer cell surface in a subject in need thereof by engaging T cells, comprising administering to the subject a pharmaceutical composition thereof of embodiment 18.

Embodiment 20 is a method of targeting one or two antigens expressed on a cancer cell surface in a subject in need thereof including using CD47 blockade induced activation of macrophage-mediated phagocytosis or CD3-mediated T cell activation, and/or treating cancer in a subject in need thereof, comprising administering to the subject a pharmaceutical composition comprising the isolated bispecific antibody or antigen-binding fragment thereof of any one of embodiments 1 to 14 and a pharmaceutically acceptable carrier, optionally, the cancer is selected from the group consisting of a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Embodiment 21 is a method of producing the bispecific antibody or antigen-binding fragment thereof of any one of embodiments 1 to 14, comprising culturing a cell comprising a nucleic acid encoding the bispecific antibody or antigen-binding fragment thereof under conditions to produce the bispecific antibody or antigen-binding fragment thereof, and recovering the bispecific antibody or antigen-binding fragment thereof from the cell or culture.

Embodiment 22 is a method of producing a pharmaceutical composition comprising the bispecific antibody or antigen-binding fragment thereof of any one of embodiments 1 to 14, comprising combining the bispecific antibody or antigen-binding fragment thereof with a pharmaceutically acceptable carrier to obtain the pharmaceutical composition.

### EXAMPLES

### Example 1: Construction of Bispecific Antibodies With Charge Pairs

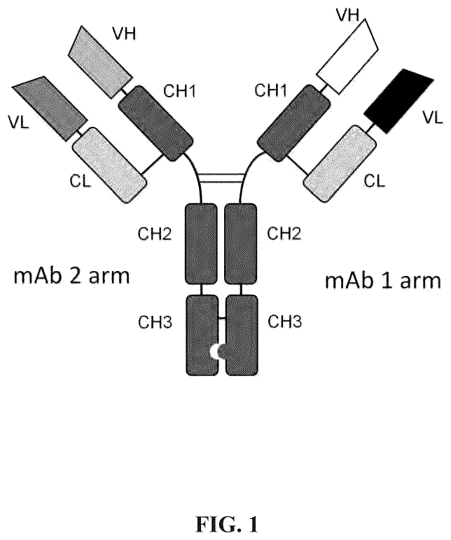

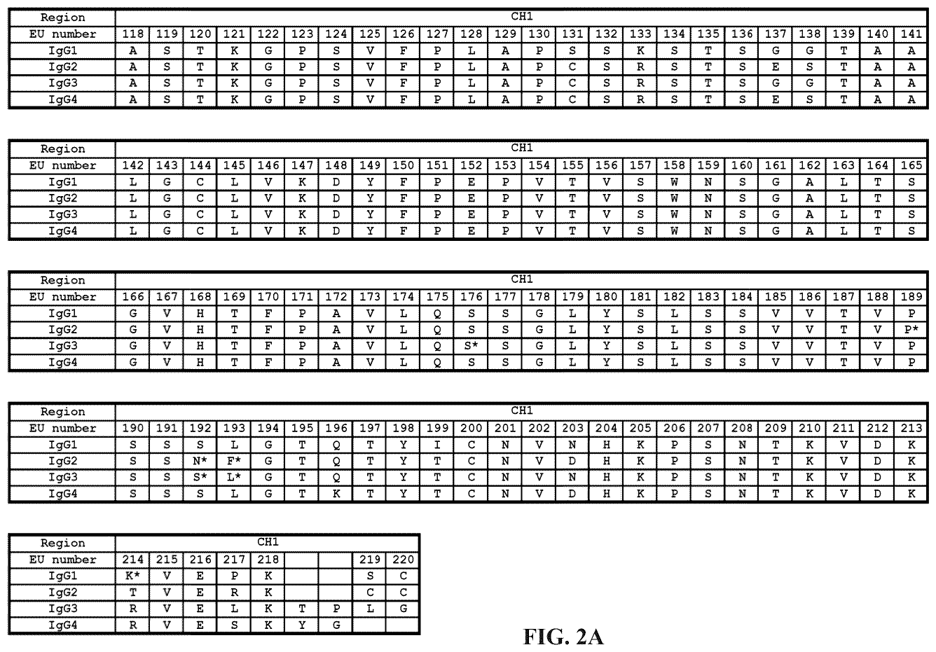

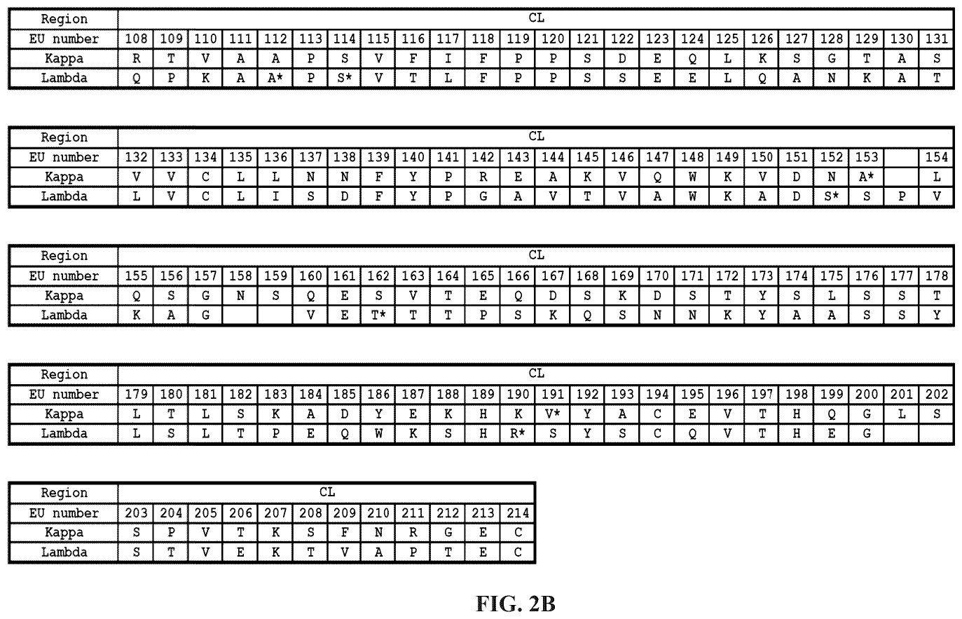

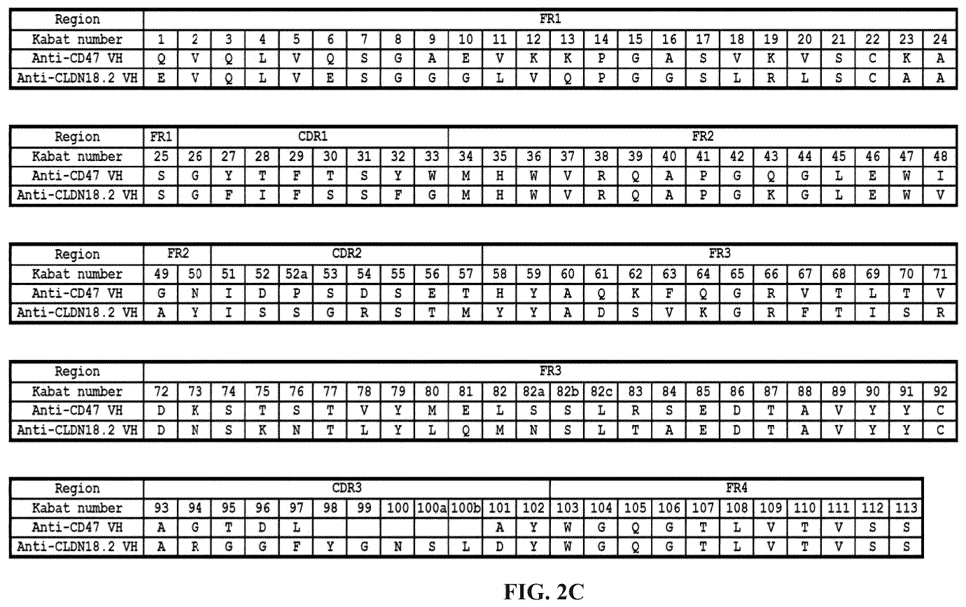

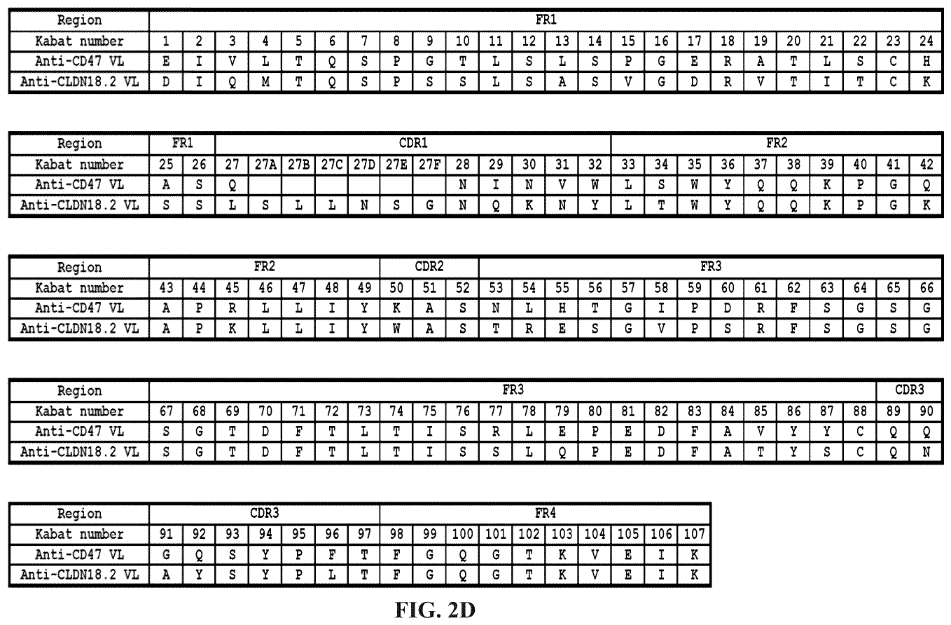

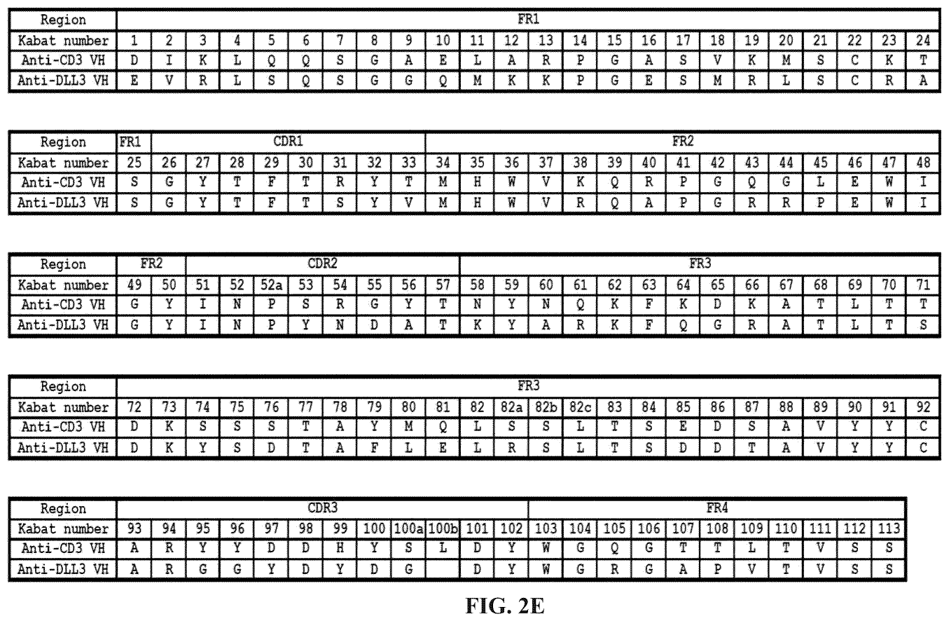

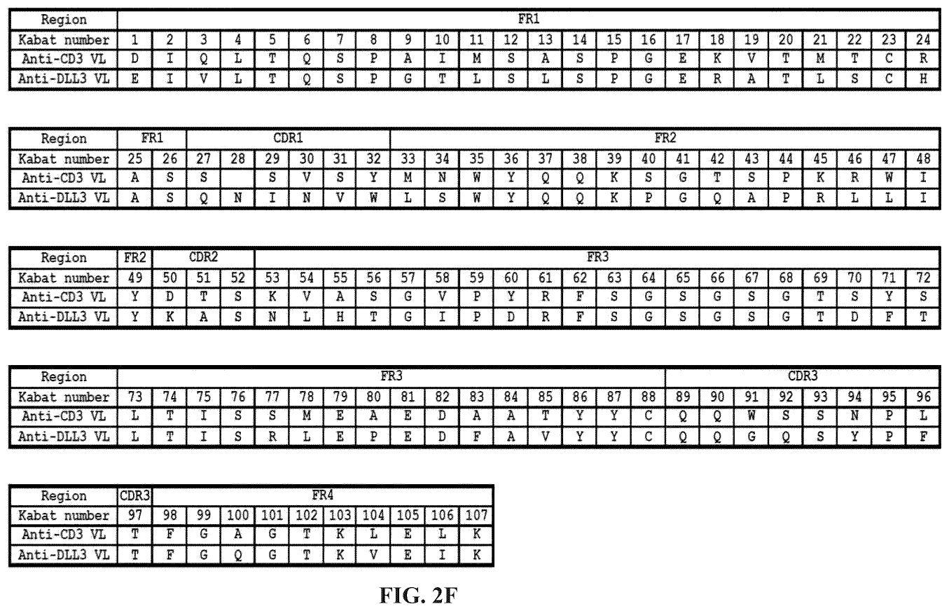

FIG. 1 illustrates a bispecific antibody (bsAb) with two different heavy chains (H1 and H2) and two different light chains (L1 and L2) in a heterodimer of H1H2, which can be facilitated with common approaches such as knob-in-hole and charge pairs. Oppositely charged amino acids can be introduced in the CH1 and CL regions of each arm of the bispecific antibody to enhance the correct cognate pairing of each heavy chain with its corresponding light chain (HIL1 and H2L2). The oppositely charged pair(s) can better differentiate the physical properties of the bsAb and potential impurities expressed by cells in the production process, facilitating the separation of the intended bsAb from its impurities on chromatography and hence purification and production of the intended bsAb, and making it possible to use the common mAb manufacturing platform (typically involving Protein A affinity, anion exchange and cation exchange chromatography steps) to manufacture bispecific antibodies. The examples below are shown with bispecific antibodies in the human IgG1 heavy chain CH1 and kappa light chain CL sequences (FIGS. 2A-2B and Table 5). This concept can also be applied to constructing bispecific antibodies using CH1 of human IgG2, IgG3, or IgG4 heavy chain (FIG. 2A and Table 5), and CL of human lambda light chain whenever conserved knock-in sites exist (FIG. 2B and Table 5). Whenever a knock-in site happens to be a native charge residue identical to the intended charge amino acid, it can be directly used to form the intended charge pairs. Designed charge pairs are listed in Table 1 (as first described in WO2021/126538, which is incorporated by reference herein in its entirety). In describing the charge pairs, G166D/E represents substitution of G at position 166 (EU numbering) with D or E, in which case G166 is the knock-in site; D170D/E represents keeping D at position 170 or substitution of D at position 170 with E; all the other substitutions follow the same naming rule. The VH and VL sequences of several mAbs used for constructing bispecific antibodies are shown in FIGS. 2C-2F and Table 2.

The anti-CD47 and the anti-CLDN18.2 mAbs (FIGS. 2C-2D and Table 2) were used to construct bispecific antibodies, to which various charge pair designs were introduced. The charge pair designs used in the anti-CD47/anti-CLDN18.2 bispecific antibodies are listed in Table 3. The VH and VL regions of the bsAb were fused to the constant regions of human IgG1 heavy chain (HC) and kappa light chain (LC), respectively. A strategy of shifting the interchain disulfide bond between the HC and the LC on the mAb 1 (anti-CD47) arm to K133 on HC and F209 on LC was employed. These steps were carried out as described in WO2021/126538, which is incorporated by reference herein in its entirety. On the backbone of human IgG1 heavy chain and kappa light chain (Kabat numbering for the VH and VL regions; EU numbering for the CH1 and CL regions) of the bsAb, the mAb 1 (anti-CD47) HC has the T366W (EU numbering for CH2 and CH3 regions) mutation to form a “knob” and the mAb 2 (anti-CDN18.2) HC has the mutations T366S, L368A, and Y407V to form a “hole,” so that the two heavy chains were favored to form a bsAb with heterodimeric HCs (mAb 1 HC/mAb 2 HC). In addition, a S354C cysteine mutation was introduced on the mAb 1 HC and a Y349C cysteine mutation was introduced on the mAb 2 HC to stabilize the heterodimeric pairing of the heavy chains of the heterodimer (Merchant et al. Nat. Biotechnol. 16(7):677-81 (1998)). The anti-CD47/anti-CLDN18.2 bispecific antibodies with different charge pair designs (Table 3) were transfected in ExpiCHO-S cells and the simultaneous expression of the two heavy chains and the two light chains in the same cell resulted in the expression and assembly of a desired bsAb and certain impurities. The bispecific antibodies were purified using Protein A affinity chromatography.

An anti-CD3 mAb and a humanized anti-DLL3 mAb (described in International Pat. Pub. No. WO2019217145, which is incorporated by reference herein in its entirety) were used to construct an anti-CD3/anti-DLL3 bsAb (FIGS. 2E-2F and Table 2). The VH and VL regions of the bsAb were fused to the constant regions of human IgG1 heavy chain (HC) and kappa light chain (LC), respectively. The charge pairs HC (Q39E, T187E)/LC (Q38K, D170K) were introduced to the anti-DLL3 arm of the bsAb; the charge pairs HC (Q39K, T187K)/LC (Q38E, D170E) were introduced to the anti-CD3 arm of the bsAb. A strategy of shifting the interchain disulfide bond between the HC and the LC on the mAb 2 (anti-DLL3) arm to K133 on HC and F209 on LC was employed to favor the expression, purification, and/or production as well as increase the stability of the intended bsAb when the two HCs and two LCs were co-transfected in transfected cells. These steps were carried out as described in WO2021/126538, which is incorporated by reference herein in its entirety. The VH, CH1, VL and CL sequences of the final bsAb (named bsAb_33) are listed in Table 4.

As described above, bsAb_33 is on the backbone of human IgG1 heavy chain and kappa light chain (Kabat numbering for the VH and VL regions; EU numbering for the CH1 and CL regions). The mAb 1 (anti-CD3) HC has the T366W (EU numbering for CH2 and CH3 regions) mutation to form a “knob” and the mAb 2 (anti-DLL3) HC has the mutations T366S, L368A, and Y407V to form a “hole,” so that the two heavy chains were favored to form a bsAb with heterodimeric HCs (mAb 1 HC/mAb 2 HC) rather than homodimeric HCs (mAb 1 HC/mAb 1 HC or mAb 2 HC/mAb 2 HC). In addition, a S354C cysteine mutation was introduced on the mAb 1 HC and a Y349C cysteine mutation was introduced on the mAb 2 HC to stabilize the heterodimeric pairing of the heavy chains of the heterodimer (Merchant et al. Nat. Biotechnol. 16(7):677-81 (1998)). Further, L234A and L235A mutations were introduced in the CH2 regions of both H1 and H2.

The bsAb bsAb_33 was transfected in ExpiCHO-S cells and the simultaneous expression of the two heavy chains and the two light chains in the same cell resulted in the expression and assembly of a desired bsAb and certain impurities. The bsAb was purified using Protein A chromatography.

TABLE 1
Amino acid residues in the CH1 and CL regions
for the formation of charge pairs

| Charge pair Name | CH1 | CL |
|---|---|---|
| G166/S114 | G166 | S114 |
| T187/D170 | T187 | D170 |
| S131/P119 | S131 | P119 |
| A129/S121 | A129 | S121 |

Note
EU numbering is used for the CH1 and CL regions.

TABLE 2
Sequences of various regions in the anti-CD47/anti-CLDN18.2 and
anti-CD3/anti-DLL3 bispecific antibodies

| | | SEQ |
|---|---|---|
| | | ID |
| Name | Sequence | NO: |
| Anti-CD47 VH | QVQLVQSGAEVKKPGASVKVSCKASGYTFTSYWMHWVRQAPGQGLEWIGN | 1 |
| | IDPSDSETHYAQKFQGRVTLTVDKSTSTVYMELSSLRSEDTAVYYCAGTD | |
| | LAYWGQGTLVTVSS | |
| Anti-CD47 VL | EIVLTQSPGTLSLSPGERATLSCHASQNINVWLSWYQQKPGQAPRLLIYK | 2 |
| | ASNLHTGIPDRFSGSGSGTDFTLTISRLEPEDFAVYYCQQGQSYPFTFGQ | |
| | GTKVEIK | |
| Anti-CLDN18.2 | EVQLVESGGGLVQPGGSLRLSCAASGFIFSSFGMHWVRQAPGKGLEWVAY | 3 |
| VH | ISSGRSTMYYADSVKGRFTISRDNSKNTLYLQMNSLTAEDTAVYYCARGG | |
| | FYGNSLDYWGQGTLVTVSS | |
| Anti-CLDN18.2 | DIQMTQSPSSLSASVGDRVTITCKSSLSLLNSGNQKNYLTWYQQKPGKAP | 4 |
| VL | KLLIYWASTRESGVPSRFSGSGSGTDFTLTISSLQPEDFATYSCQNAYSY | |
| | PLTFGQGTKVEIK | |
| Anti-CD3 VH | DIKLQQSGAELARPGASVKMSCKTSGYTFTRYTMHWVKQRPGQGLEWIGY | 5 |
| | INPSRGYTNYNQKFKDKATLTTDKSSSTAYMQLSSLTSEDSAVYYCARYY | |
| | DDHYSLDYWGQGTTLTVSS | |
| Anti-CD3 VL | DIQLTQSPAIMSASPGEKVTMTCRASSSVSYMNWYQQKSGTSPKRWIYDT | 6 |
| | SKVASGVPYRFSGSGSGTSYSLTISSMEAEDAATYYCQQWSSNPLTFGAG | |
| | TKLELK | |
| Anti-DLL3 VH | EVRLSQSGGQMKKPGESMRLSCRASGYTFTSYVMHWVRQAPGRRPEWIGY | 7 |
| | INPYNDATKYARKFQGRATLTSDKYSDTAFLELRSLTSDDTAVYYCARGG | |
| | YDYDGDYWGRGAPVTVSS | |
| Anti-DLL3 VL | EIVLTQSPGTLSLSPGERATLSCHASQNINVWLSWYQQKPGQAPRLLIYK | 8 |
| | ASNLHTGIPDRFSGSGSGTDFTLTISRLEPEDFAVYYCQQGQSYPFTFGQ | |
| | GTKVEIK | |

TABLE 3
Charge pair designs in anti-CD47/anti-
CLDN18.2 bispecific antibodies

| | | Muta- | Muta- | Muta- | Muta- |
|---|---|---|---|---|---|
| | | tions | tions | tions | tions |
| BsAb | Arm | in VH | in VL | in CH1 | in CL |
| WT | Anti-CD47 | | | | |
| | Anti-CLDN18.2 | | | | |
| PAC12 | Anti-CD47 | | | C220S; | C214S; |
| | | | | K133C | F209C |
| | Anti-CLDN18.2 | | | | |
| ek | Anti-CD47 | Q39E | Q38K | C220S; | C214S; |
| | | | | K133C | F209C |
| | Anti-CLDN18.2 | Q39K | Q38E | | |
| ke | Anti-CD47 | Q39K | Q38E | C220S; | C214S; |
| | | | | K133C | F209C |
| | Anti-CLDN18.2 | Q39E | Q38K | | |
| EK (gs) | Anti-CD47 | | | C220S; | C214S; |
| | | | | K133C; | F209C; |
| | | | | G166E | S114K |
| | Anti-CLDN18.2 | | | G166K | S114E |
| EK (td) | Anti-CD47 | | | C220S; | C214S; |
| | | | | K133C; | F209C; |
| | | | | T187E | D170K |
| | Anti-CLDN18.2 | | | T187K | D170E |
| EK (5) | Anti-CD47 | | | C220S; | C214S; |
| | | | | K133C; | F209C; |
| | | | | S131E | P119K |
| | Anti-CLDN18.2 | | | S131K | P119E |
| EK (9) | Anti-CD47 | | | C220S; | C214S; |
| | | | | K133C; | F209C; |
| | | | | A129E | S121K |
| | Anti-CLDN18.2 | | | A129K | S121E |
| KE (gs) | Anti-CD47 | | | C220S; | C214S; |
| | | | | K133C; | F209C; |
| | | | | G166K | S114E |
| | Anti-CLDN18.2 | | | G166E | S114K |
| KE (td) | Anti-CD47 | | | C220S; | C214S; |
| | | | | K133C; | F209C; |
| | | | | T187K | D170E |
| | Anti-CLDN18.2 | | | T187E | D170K |
| KE (5) | Anti-CD47 | | | C220S; | C214S; |
| | | | | K133C; | F209C; |
| | | | | S131K | P119E |
| | Anti-CLDN18.2 | | | S131E | P119K |
| KE (9) | Anti-CD47 | | | C220S; | C214S; |
| | | | | K133C; | F209C; |
| | | | | A129K | S121E |
| | Anti-CLDN18.2 | | | A129E | S121K |
| ekEK (gs) | Anti-CD47 | Q39E | Q38K | C220S; | C214S; |
| | | | | K133C; | F209C; |
| | | | | G166E | S114K |
| | Anti-CLDN18.2 | Q39K | Q38E | G166K | S114E |
| ekEK (td) | Anti-CD47 | Q39E | Q38K | C220S; | C214S; |
| | | | | K133C; | F209C; |
| | | | | T187E | D170K |
| | Anti-CLDN18.2 | Q39K | Q38E | T187K | D170E |
| ekEK (5) | Anti-CD47 | Q39E | Q38K | C220S; | C214S; |
| | | | | K133C; | F209C; |
| | | | | S131E | P119K |
| | Anti-CLDN18.2 | Q39K | Q38E | S131K | P119E |
| ekEK (9) | Anti-CD47 | Q39E | Q38K | C220S; | C214S; |
| | | | | K133C; | F209C; |
| | | | | A129E | S121K |
| | Anti-CLDN18.2 | Q39K | Q38E | A129K | S121E |
| keKE (gs) | Anti-CD47 | Q39K | Q38E | C220S; | C214S; |
| | | | | K133C; | F209C; |
| | | | | G166K | S114E |
| | Anti-CLDN18.2 | Q39E | Q38K | G166E | S114K |
| keKE (td) | Anti-CD47 | Q39K | Q38E | C220S; | C214S; |
| | | | | K133C; | F209C; |
| | | | | T187K | D170E |
| | Anti-CLDN18.2 | Q39E | Q38K | T187E | D170K |
| keKE (5) | Anti-CD47 | Q39K | Q38E | C220S; | C214S; |
| | | | | K133C; | F209C; |
| | | | | S131K | P119E |
| | Anti-CLDN18.2 | Q39E | Q38K | S131E | P119K |
| keKE (9) | Anti-CD47 | Q39K | Q38E | C220S; | C214S; |
| | | | | K133C; | F209C; |
| | | | | A129K | S121E |
| | Anti-CLDN18.2 | Q39E | Q38K | A129E | S121K |

Note:
bsAb, bispecific antibody.
Kabat numbering is used for VH and VL regions; EU numbering is used for the CH1 and CL regions; blank, no mutation was introduced.

TABLE 4
Sequences of various regions in the anti-CD3/anti-DLL3
bispecific antibody bsAb_33

| | | SEQ |
|---|---|---|
| | | ID |
| Name | Sequence | NO: |
| Anti-CD3 | DIKLQQSGAELARPGASVKMSCKTSGYTFTRYTMHWVKKRPGQGLEW | 9 |
| VH_33 | IGYINPSRGYTNYNQKFKDKATLTTDKSSSTAYMQLSSLTSEDSAVY | |
| | YCARYYDDHYSLDYWGQGTTLTVSS | |
| Anti-CD3 | ASTKGPSVFPLAPSSKSTSGGTAALGCLVKDYFPEPVTVSWNSGALT | 10 |
| CH1_33 | SGVHTFPAVLQSSGLYSLSSVVKVPSSSLGTQTYICNVNHKPSNTKV | |
| | DKKVEPKSC | |
| Anti-CD3 | DIQLTQSPAIMSASPGEKVTMTCRASSSVSYMNWYQEKSGTSPKRWI | 11 |
| VL_33 | YDTSKVASGVPYRFSGSGSGTSYSLTISSMEAEDAATYYCQQWSSNP | |
| | LTFGAGTKLELK | |
| Anti-CD3 | RTVAAPSVFIFPPSDEQLKSGTASVVCLLNNFYPREAKVQWKVDNAL | 12 |
| CL_33 | QSGNSQESVTEQDSKESTYSLSSTLTLSKADYEKHKVYACEVTHQGL | |
| | SSPVTKSFNRGEC | |
| Anti-DLL3 | EVRLSQSGGQMKKPGESMRLSCRASGYTFTSYVMHWVREAPGRRPEW | 13 |
| VH_33 | IGYINPYNDATKYARKFQGRATLTSDKYSDTAFLELRSLTSDDTAVY | |
| | YCARGGYDYDGDYWGRGAPVTVSS | |
| Anti-DLL3 | ASTKGPSVFPLAPSSCSTSGGTAALGCLVKDYFPEPVTVSWNSGALT | 14 |
| CH1_33 | SGVHTFPAVLQSSGLYSLSSVVEVPSSSLGTQTYICNVNHKPSNTKV | |
| | DKKVEPKSS | |
| Anti-DLL3 | EIVLTQSPGTLSLSPGERATLSCHASQNINVWLSWYQKKPGQAPRLL | 15 |
| VL_33 | IYKASNLHTGIPDRFSGSGSGTDFTLTISRLEPEDFAVYYCQQGQSY | |
| | PFTFGQGTKVEIK | |
| Anti-DLL3 | RTVAAPSVFIFPPSDEQLKSGTASVVCLLNNFYPREAKVQWKVDNAL | 16 |
| CL_33 | QSGNSQESVTEQDSKKSTYSLSSTLTLSKADYEKHKVYACEVTHQGL | |
| | SSPVTKSCNRGES | |

Note:
The residues resulting from amino acid substitutions are in bold and underlined form.

TABLE 5
Sequences of IgG1 CH1, IgG2 CH1, IgG3 CH1, IgG4 CH1,
Kappa CL and Lambda CL regions

| | | SEQ |
|---|---|---|
| Region | | ID |
| name | Sequence | NO: |
| IgG1 CH1 | ASTKGPSVFPLAPSSKSTSGGTAALGCLVKDYFPEPVTVSWNSGALTSGV | 17 |
| | HTFPAVLQSSGLYSLSSVVTVPSSSLGTQTYICNVNHKPSNTKVDKKVEP | |
| | KSC | |
| IgG2 CH1 | ASTKGPSVFPLAPCSRSTSESTAALGCLVKDYFPEPVTVSWNSGALTSGV | 18 |
| | HTFPAVLQSSGLYSLSSVVTVPSSNFGTQTYTCNVDHKPSNTKVDKTVER | |
| | KCC | |
| IgG3 CH1 | ASTKGPSVFPLAPCSRSTSGGTAALGCLVKDYFPEPVTVSWNSGALTSGV | 19 |
| | HTFPAVLQSSGLYSLSSVVTVPSSSLGTQTYTCNVNHKPSNTKVDKRVEL | |
| | KTPLG | |
| IgG4 CH1 | ASTKGPSVFPLAPCSRSTSESTAALGCLVKDYFPEPVTVSWNSGALTSGV | 20 |
| | HTFPAVLQSSGLYSLSSVVTVPSSSLGTKTYTCNVDHKPSNTKVDKRVES | |
| | KYG | |
| Kappa CL | RTVAAPSVFIFPPSDEQLKSGTASVVCLLNNFYPREAKVQWKVDNALQSG | 21 |
| | NSQESVTEQDSKDSTYSLSSTLTLSKADYEKHKVYACEVTHQGLSSPVTK | |
| | SFNRGEC | |
| Lambda | QPKAAPSVTLFPPSSEELQANKATLVCLISDFYPGAVTVAWKADSSPVKA | 22 |
| CL | GVETTTPSKQSNNKYAASSYLSLTPEQWKSHRSYSCQVTHEGSTVEKTVA | |
| | PTEC | |

### Example 2: Properties of the Anti-CD47/Anti-CLDN18.2 Bispecific Antibodies With Charge Pairs

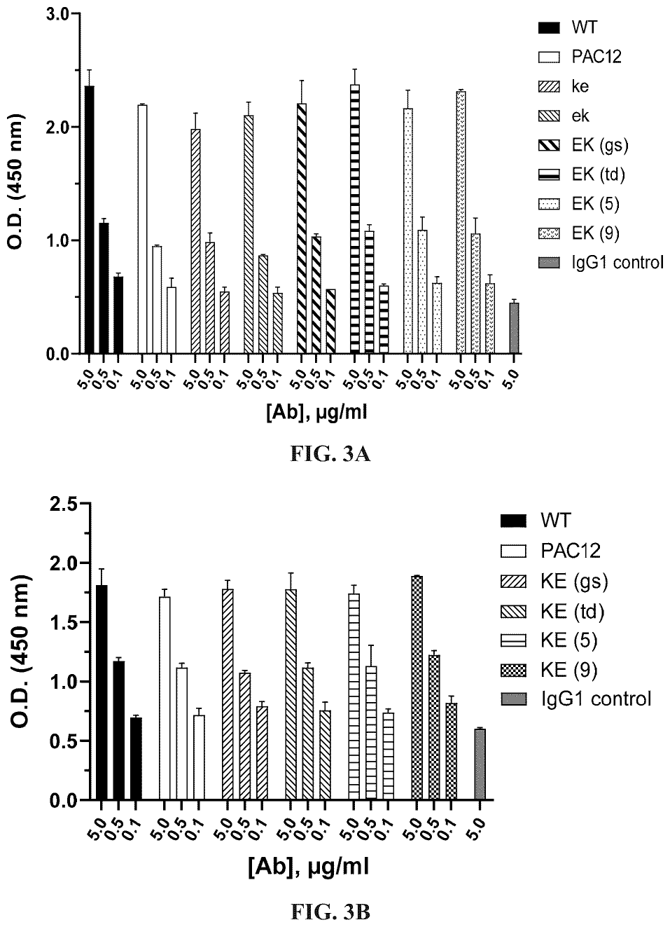

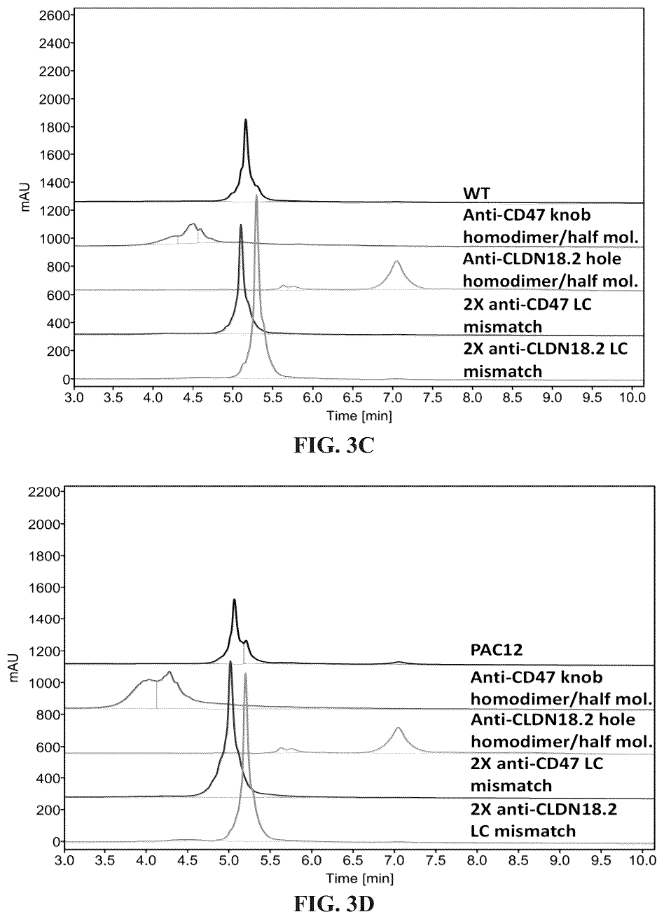

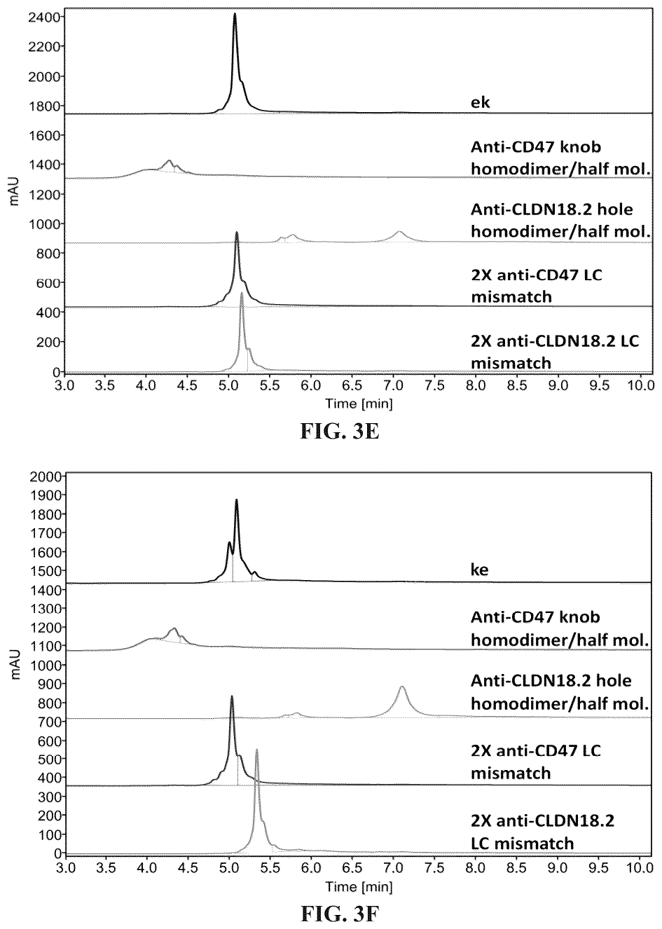

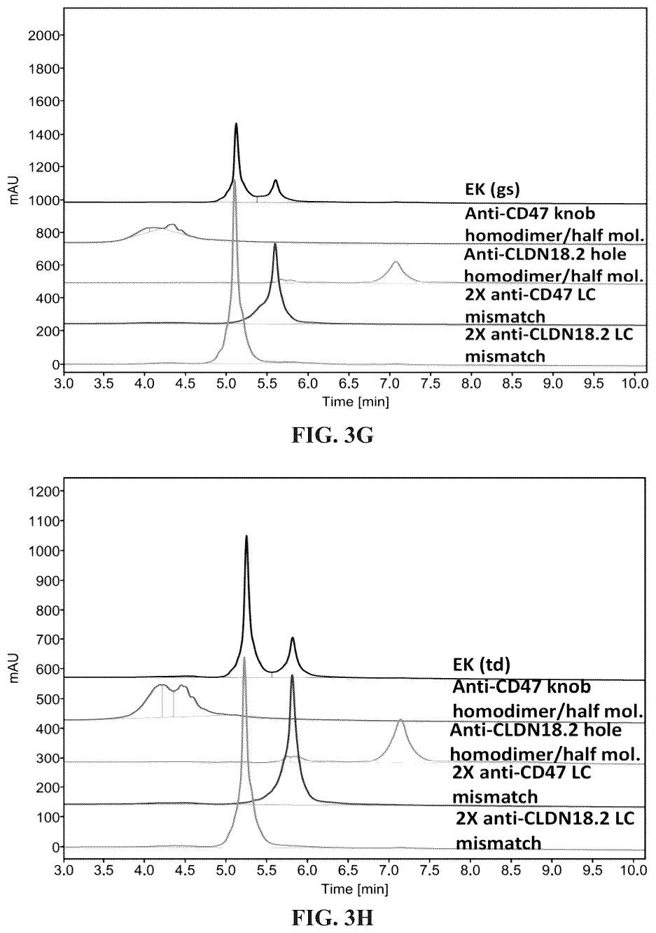

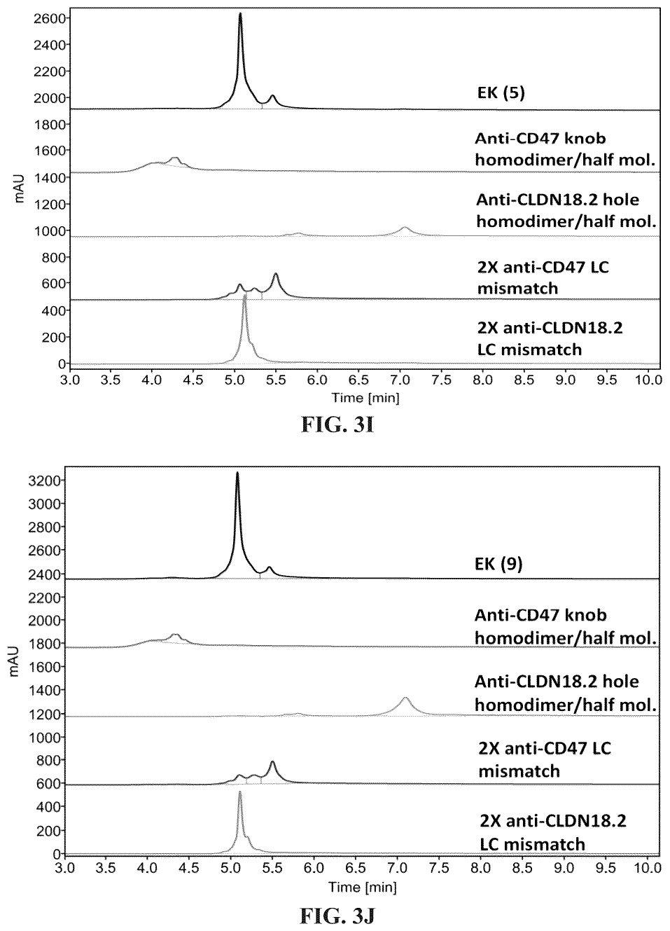

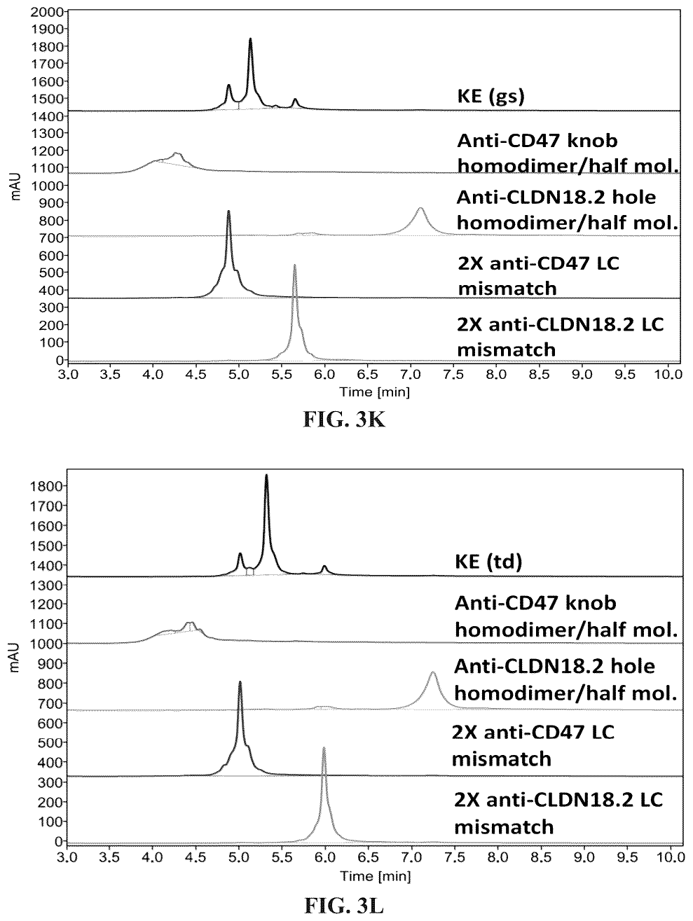

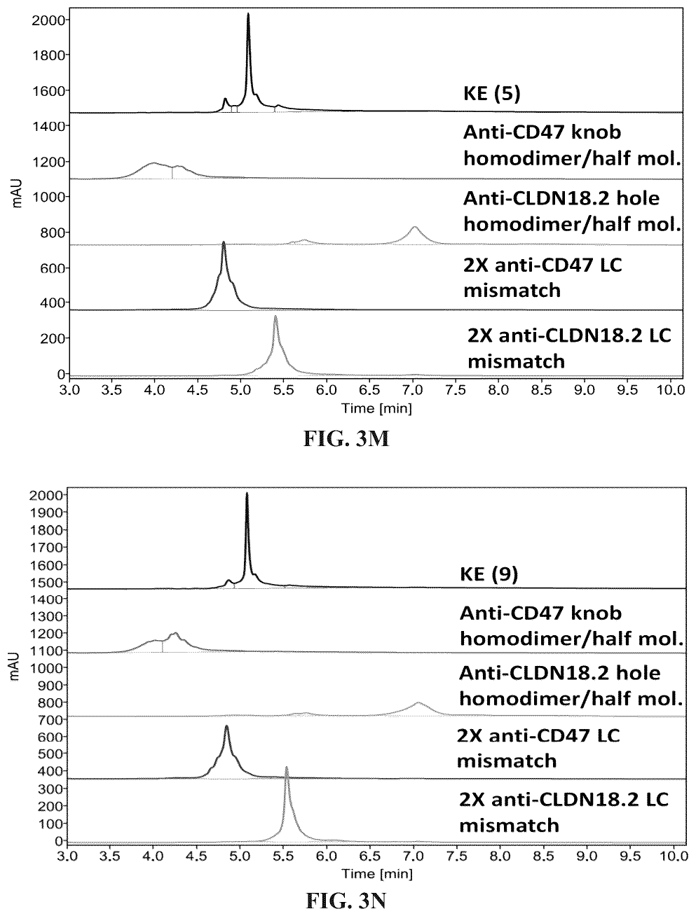

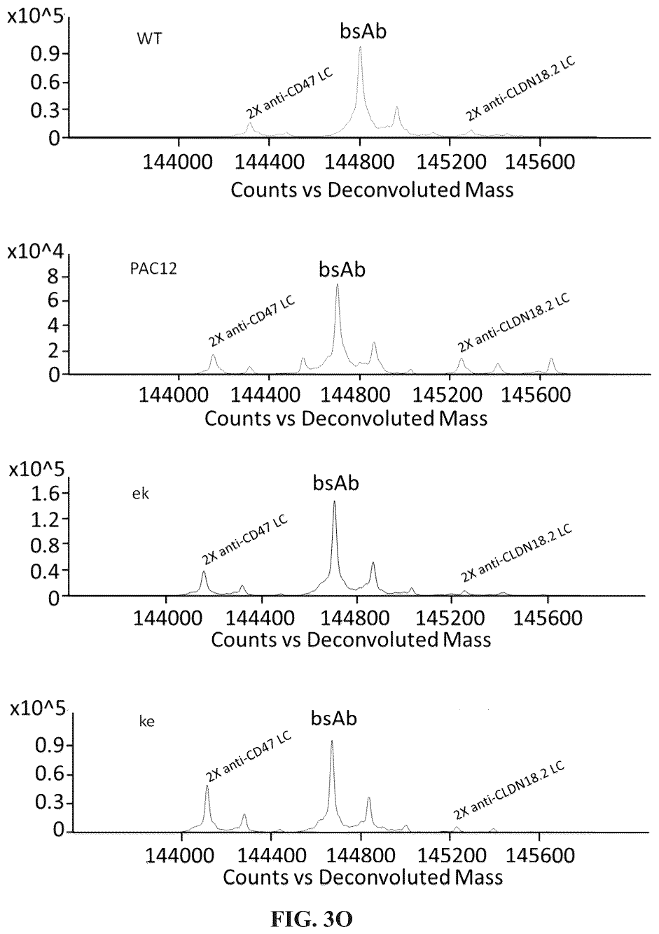

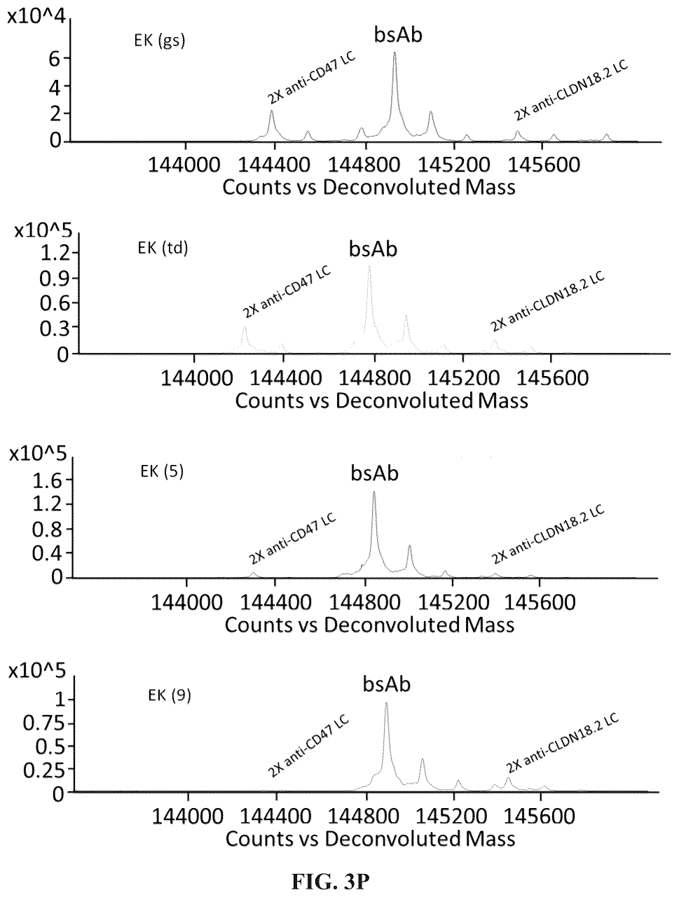

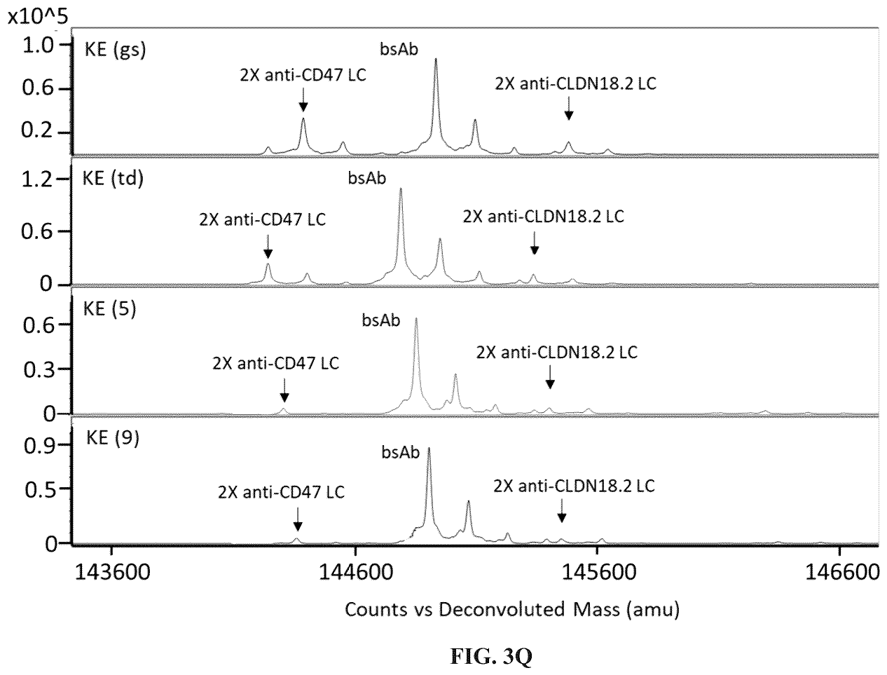

The anti-CD47/anti-CLDN18.2 bispecific antibodies with one pair of charge pair in each of them were produced in ExpiCHO-S cells by transfecting the DNAs of both arms at 1:1 ratio (standard ratio). WT and PAC12 (Table 3) were used as controls. The Protein A purified samples from these transfections were analyzed in a bridging ELISA assay to confirm the formation of the intended bispecific antibodies (FIGS. 3A-3B). The bridging ELISA assay was carried out using ExpiCHO-S cells stably expressing human CLDN18.2 on a plate to capture the bsAb. Then biotinylated human CD47 protein was added to bind to the captured bsAb and the detection was carried out using HRP-conjugated streptavidin. The Protein A purified samples were also analyzed on CEX-HPLC along with their major impurity standards (FIGS. 3C-3N). The CEX-HPLC was run using a linear salt gradient [buffer A: 25 mM sodium phosphate (pH 6.0) and 2% acetonitrile; buffer B: 25 mM sodium phosphate (pH 6.0), 1 M NaCl and 2% acetonitrile]. Compared with WT, PAC12, ek or ke samples (FIGS. 3C-3F), the 2×anti-CD47 LC mismatch impurity was separated from the main peak in the “EK” [(including EK (gs), EK (td), EK (5), and EK (9); Table 3] bsAb samples (FIGS. 3G-3J). Further, both the 2×anti-CD47 and 2×anti-CLDN18.2 LC mismatch impurities were separated from the main peak in the “KE” [(including KE (gs), KE (td), KE (5), and KE (9); Table 3] bsAb samples (FIG. 3K-3N). These data indicate that the “EK” and “KE” charge pairs can differentiate the physical properties of one or both of the LC mismatch impurities from that of the bsAb on cation exchange (CEX) chromatography using a linear salt gradient and can facilitate the separation of the bsAb from one or both of the LC mismatch impurities on CEX chromatography. This property can facilitate the purification and production of the bsAb in the manufacturing setting. The Protein A purified samples were analyzed on liquid chromatography/mass spectrometry (LC/MS). The results indicate that the intended bsAb was present in each sample and both the 2×anti-CD47 and 2×anti-CLDN18.2 LC mismatch impurities were detected in these samples (FIGS. 3O-3Q) except that in EK(9), the 2×anti-CD47 LC mismatch was almost non-detectable (FIG. 3P).

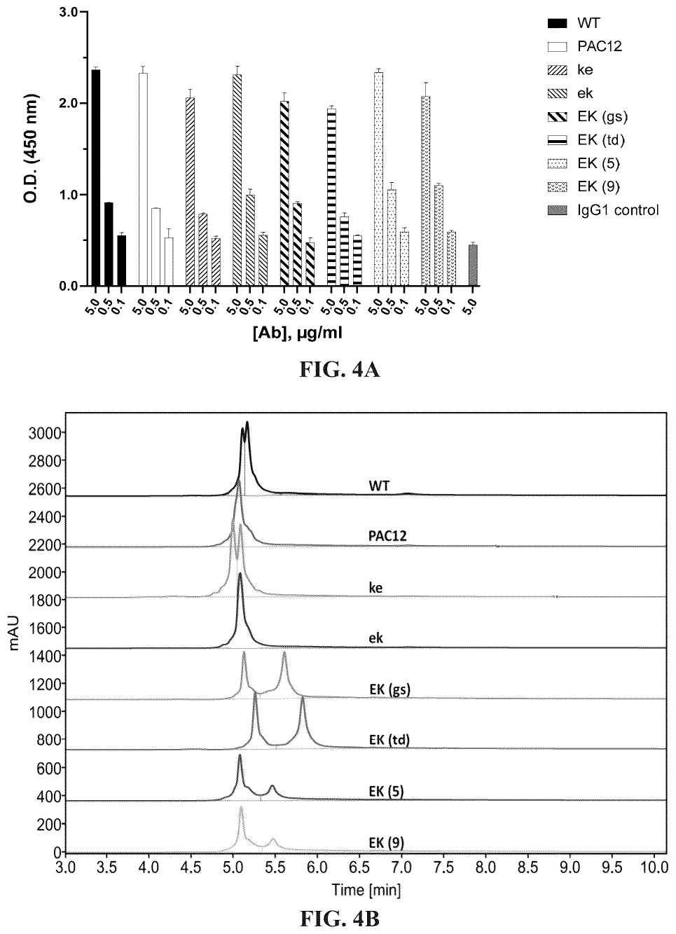

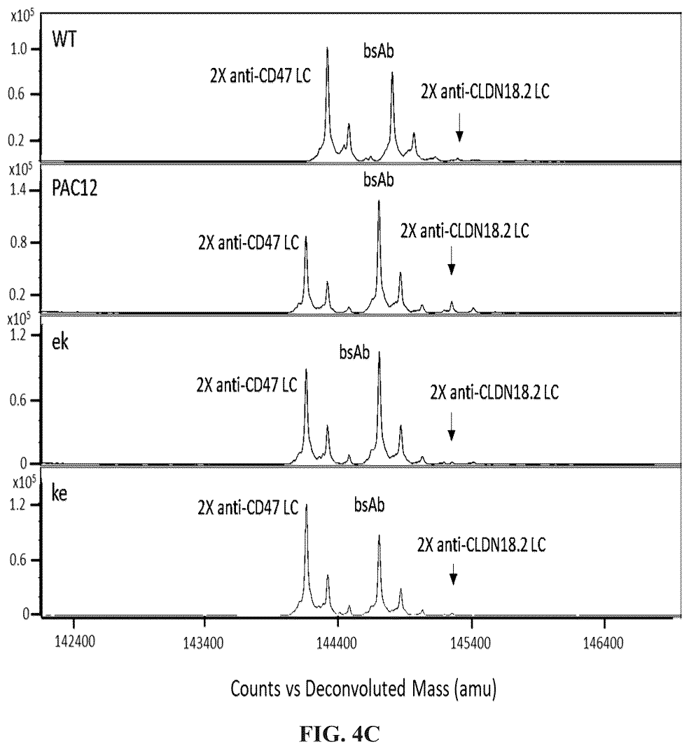

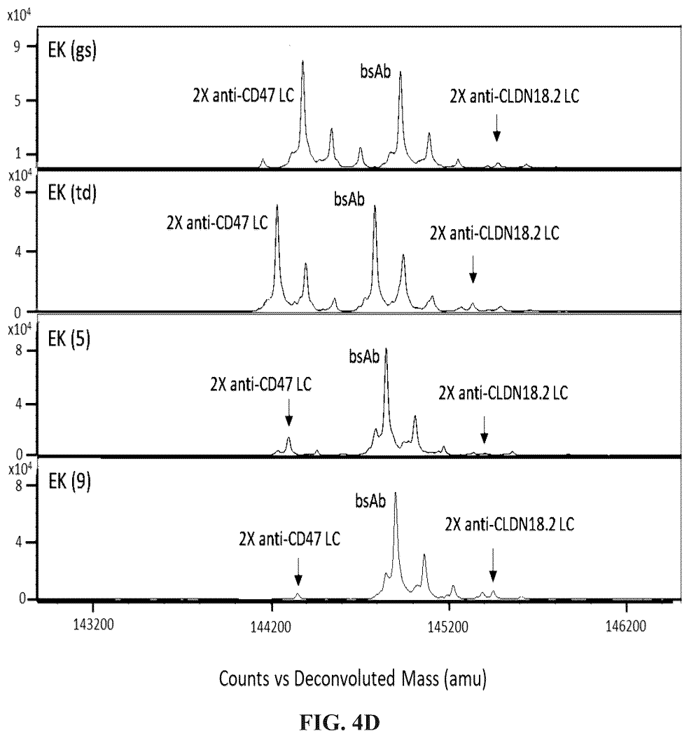

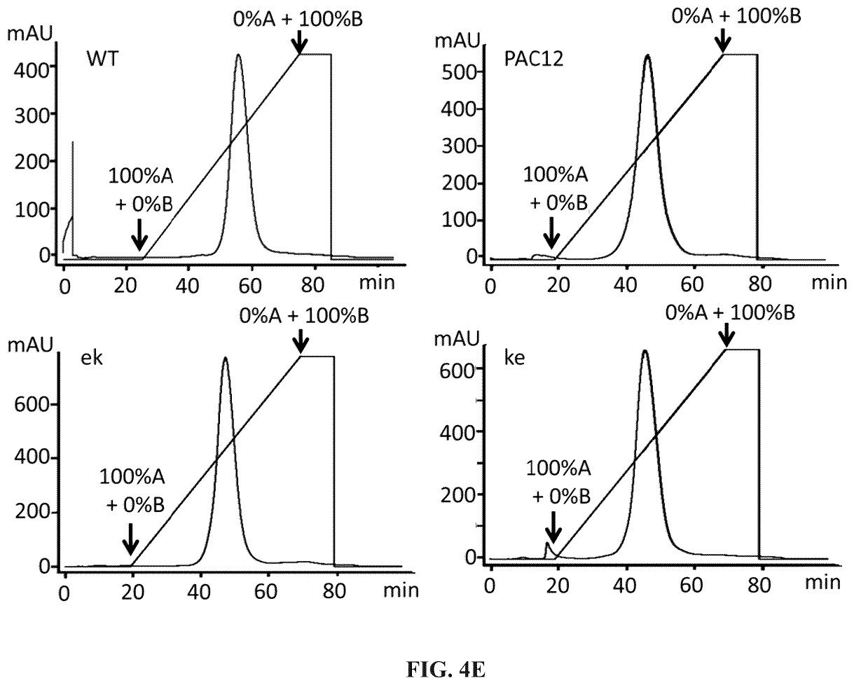

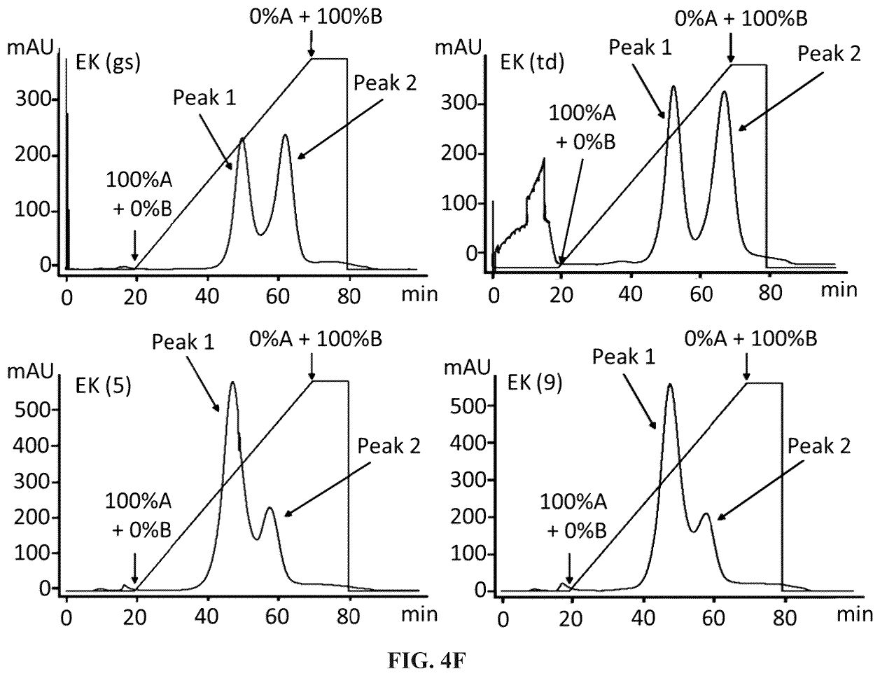

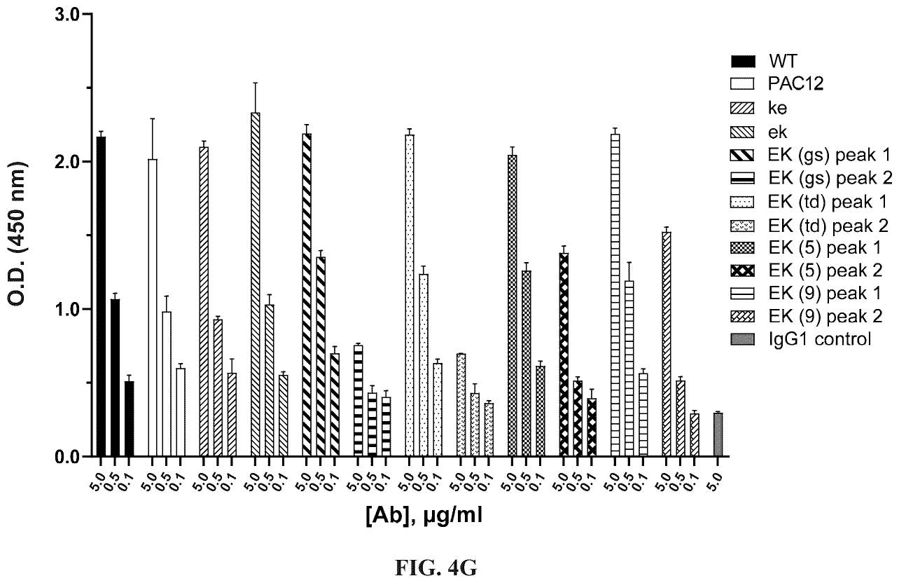

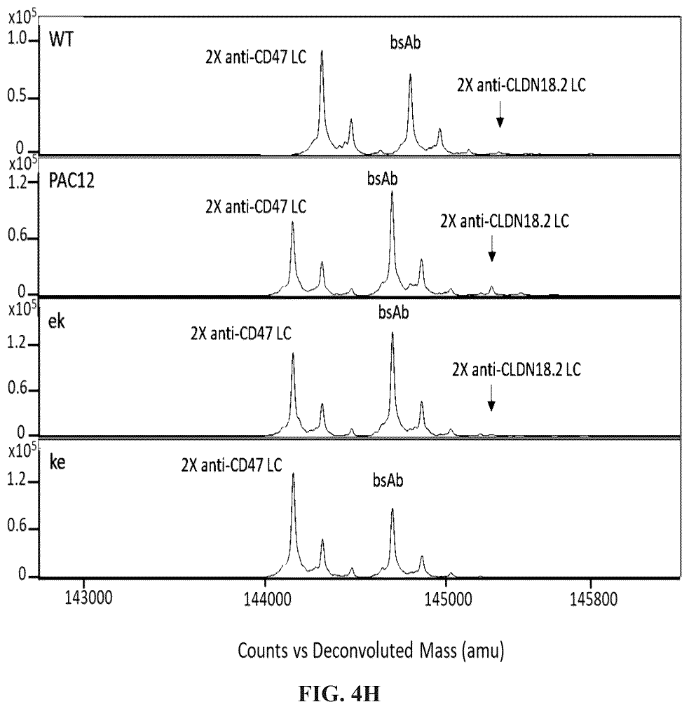

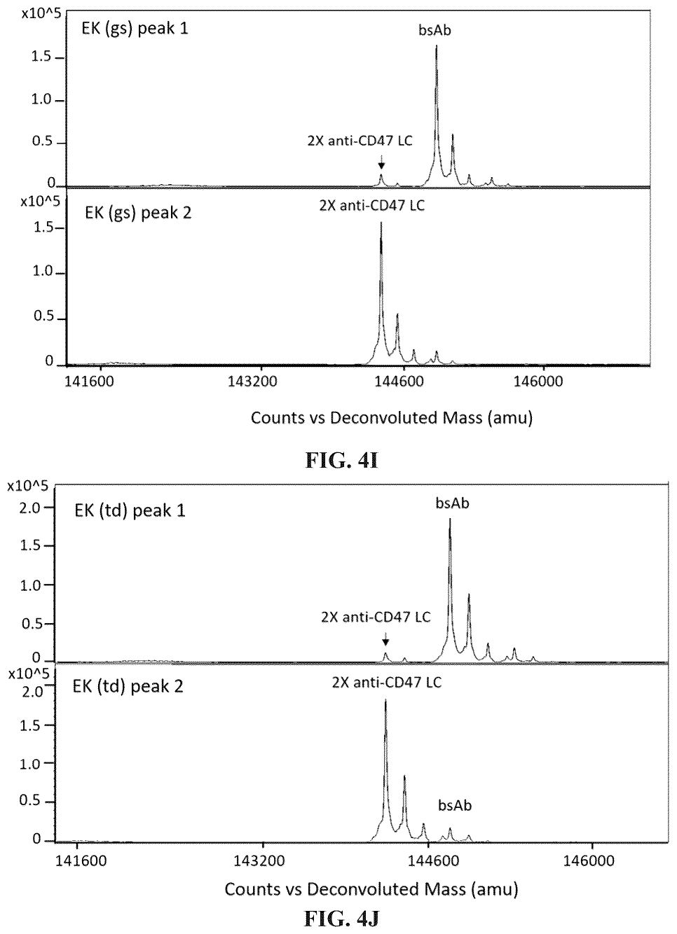

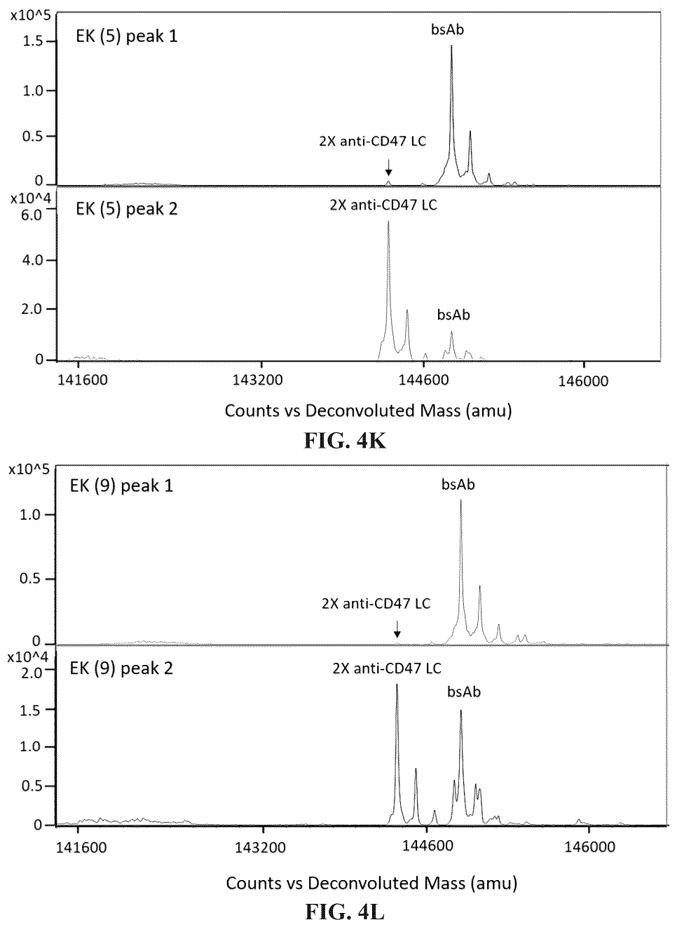

The anti-CD47/anti-CLDN18.2 bispecific antibodies with one pair of charge pair of the “EK” charge pairs in each of them (Table 3) were produced in ExpiCHO-S cells by transfecting the anti-CD47 LC DNA and anti-CLDN18.2 LC DNA at a ratio of 2:1 (anti-CD47 LC DNA : anti-CLDN18.2 LC DNA =2:1; biased DNA ratio). WT and PAC12 (Table 3) were used as controls. The Protein A purified samples from the transfections with the “EK” bispecific antibodies were analyzed in the bridging ELISA assay to confirm the formation of the intended bispecific antibodies (FIG. 4A). The Protein A purified samples were also analyzed on CEX-HPLC along with their major impurity standards using a linear salt gradient as described above. Consistent with the transfection at a biased DNA ratio for the two different LCs, relatively more 2×anti-CD47 LC mismatch impurity was produced (FIG. 4B). The existence of the major impurity species was analyzed using LC/MS (FIGS. 4C-4D). The Protein A purified samples were separated on CEX-FPLC using a linear pH gradient [buffer A: 25 mM sodium phosphate (pH 5.8) and 50 mM NaCl; buffer B: 25 mM sodium phosphate (pH 8.0) and 50 mM NaCl]. Unlike the WT, PAC12, ek or ke samples which appeared as a single peak (FIG. 4E), the “EK” samples had separation of the main peak (peak 1 where the bsAb is present) and one LC mismatch impurity species (FIG. 4F). The collected fractions from the peaks were analyzed using the same bridging ELISA assay described above (FIG. 4G). The results indicate that the bsAb is in peak 1 of each sample (FIG. 4G). Unlike the WT, PAC12, ek and ke samples in which there was no separation of the bsAb from the 2×anti-CD47 LC mismatch impurity after CEX-FPLC (FIG. 4H), the peak 1 in each of the CEX-FPLC purified “EK” samples had the intended bsAb as the major product (FIGS. 4I-4L). These data demonstrate that, by introducing the charge pairs to the “EK” bsAb designs, the main bsAb can be separated from the 2×anti-CD47 LC mismatch impurity on CEX-FPLC using a linear pH gradient, indicating that the charge pairs have superior properties in facilitating the purification of the bsAbs.

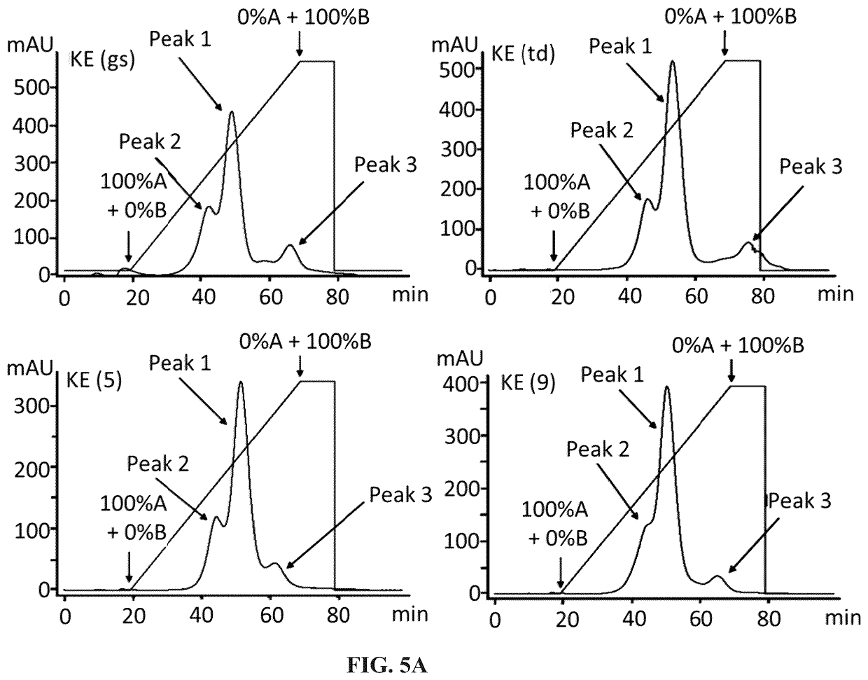

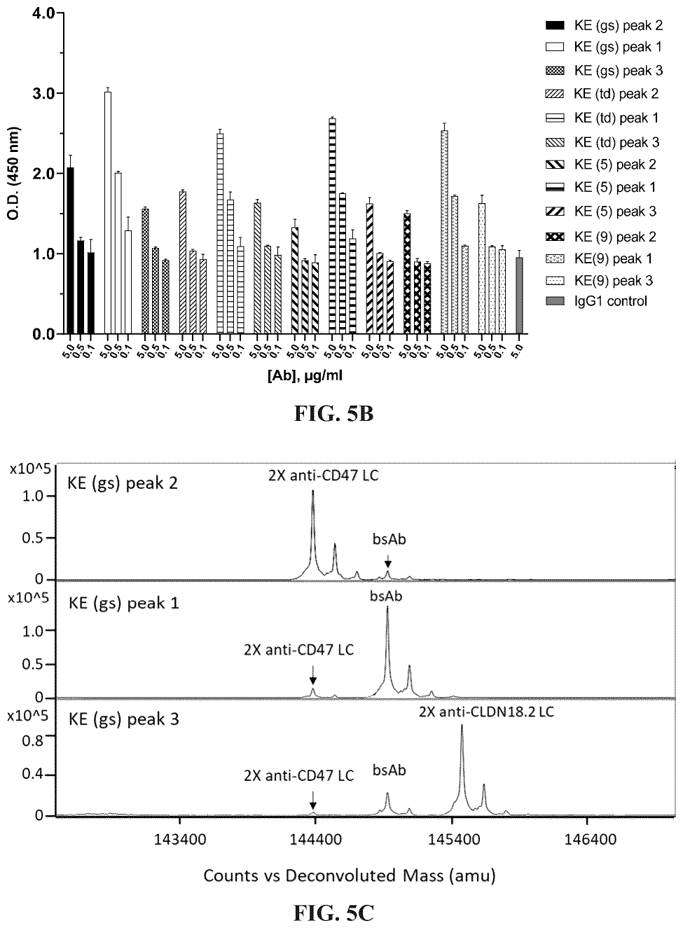

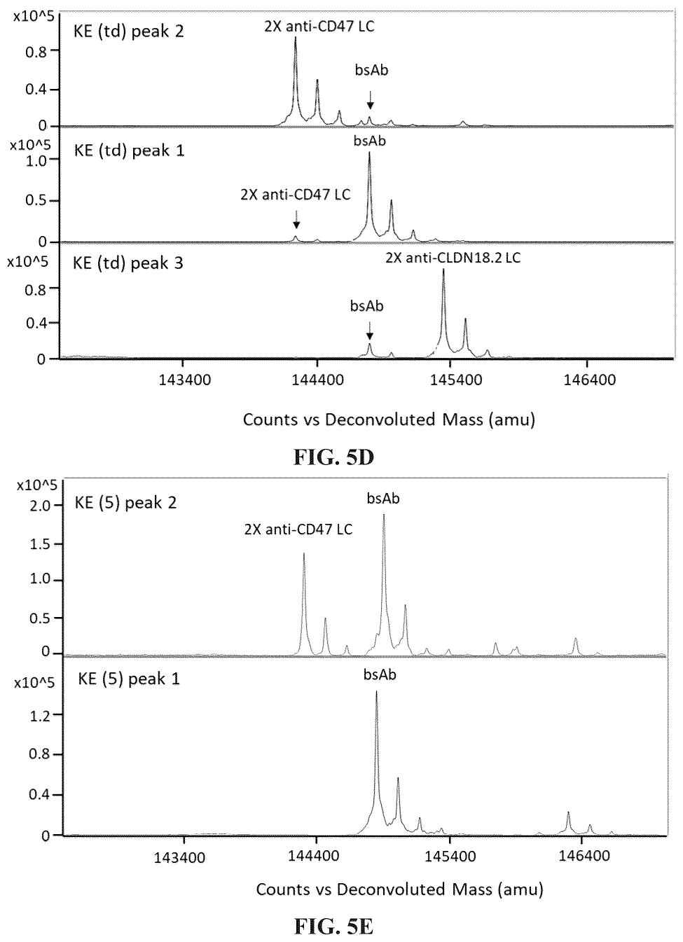

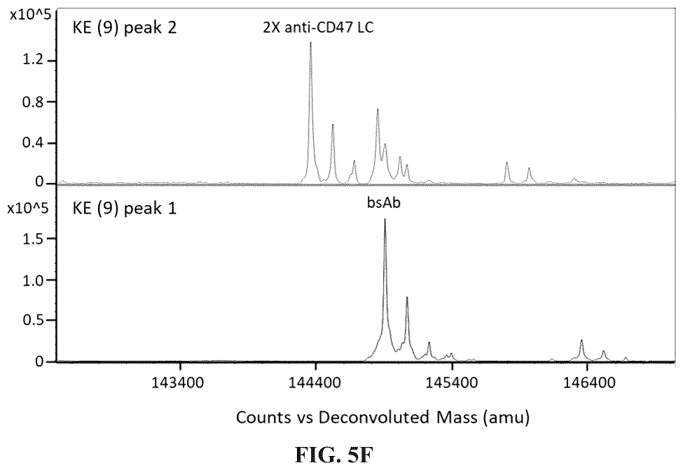

As described above, the Protein A purified bsAb samples with the “KE” charge pairs from a transfection using standard DNA ratio were analyzed using the bridging ELISA assay (FIG. 3B), CEX-HPLC (FIGS. 3K-3N), and LC/MS (FIG. 3Q). These samples were also separated on CEX-FPLC using a linear pH gradient [buffer A: 25 mM sodium phosphate (pH 5.8) and 50 mM NaCl; buffer B: 25 mM sodium phosphate (pH 8.0) and 50 mM NaCl]. Unlike the WT, PAC12, ek or ke samples which appeared as a single peak (FIG. 4E), the “KE” samples had separation of the main peak (peak 1 where the bsAb is present) and both LC mismatch impurity species (FIG. 5A). The collected fractions from the peaks were analyzed using the bridging ELISA assay (FIG. 5B) and LC/MS (FIGS. 5C-5F). The results indicate that the bsAb is in peak 1 of each sample (FIG. 5A). Unlike the WT, PAC12, ek and ke samples in which there was no separation of the bsAb from the 2×anti-CD47 LC mismatch impurity after CEX-FPLC (FIG. 4H), the peak 1 in each of the CEX-FPLC purified “KE” samples had the intended bsAb as the major product (FIGS. 5C-5F). These data demonstrate that, by introducing the charge pairs to the “KE” bsAb designs, the main bsAb can be separated from the 2×anti-CD47 LC and the 2×anti-CLDN18.2 LC mismatch impurities on CEX-FPLC using a linear pH gradient, indicating that the charge pairs have superior properties in facilitating the purification of the bsAbs.

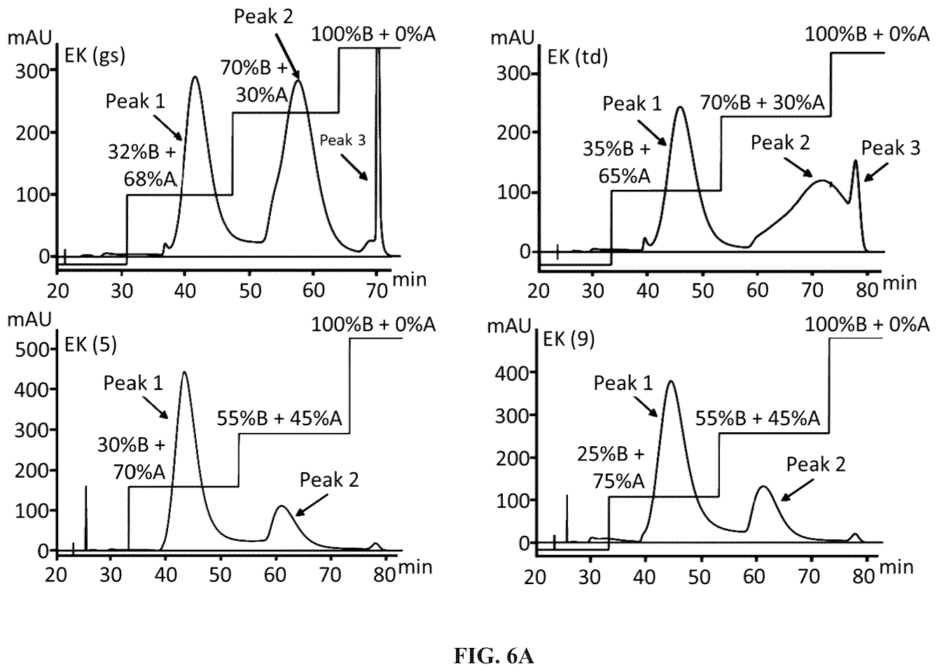

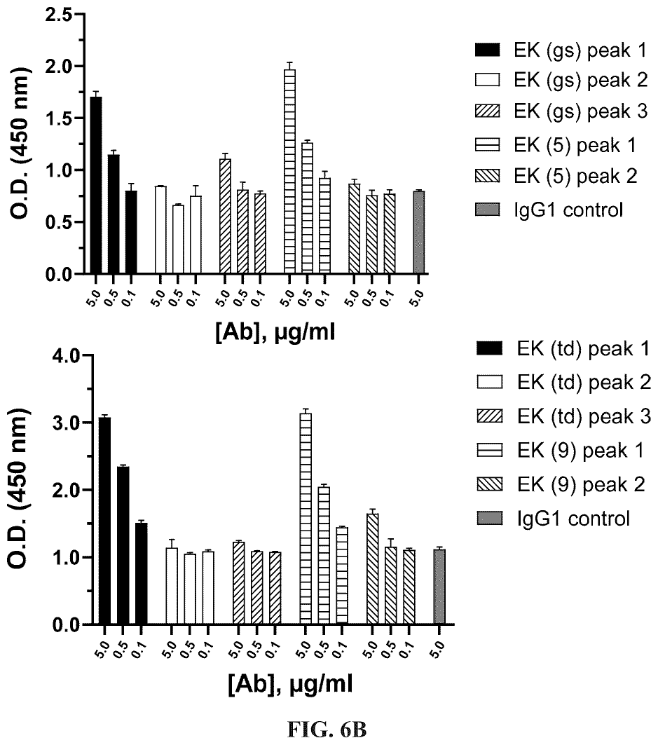

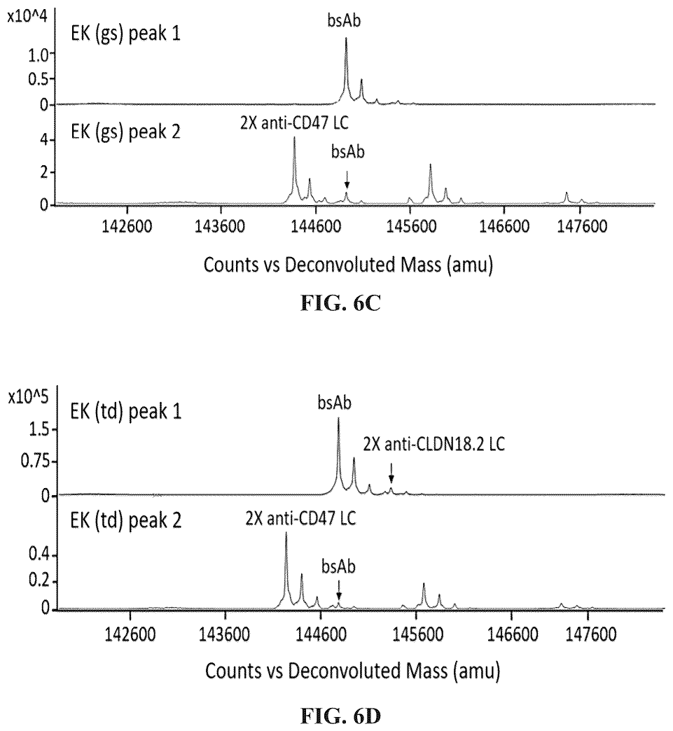

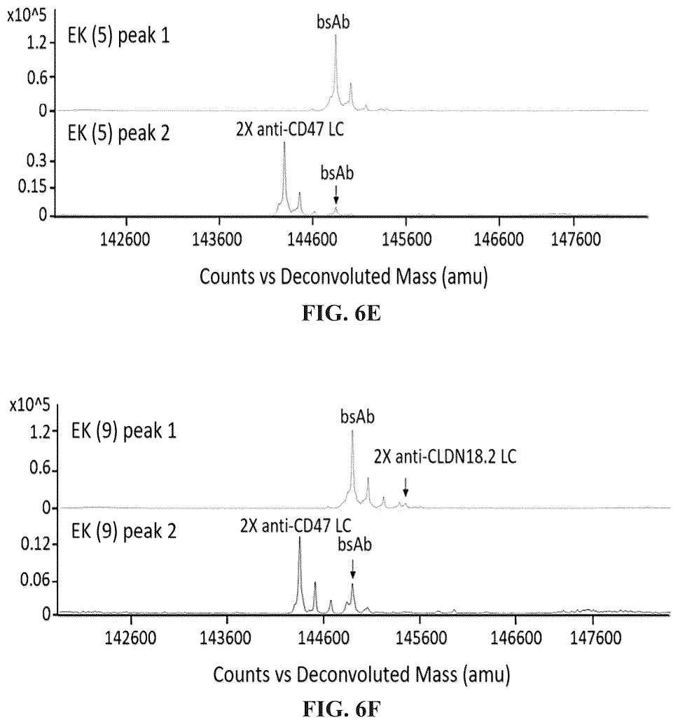

The Protein A purified “EK” bsAb samples from the transfection with biased DNA ratio were purified using stepwise elution methods [buffer A: 25 mM sodium phosphate (pH 5.8) and 80 mM NaCl; buffer B: 25 mM sodium phosphate (pH 8.0) and 40 mM NaCl]. The method was optimized for each bsAb sample. The “EK” bispecific antibodies were purified with the main bsAb peak (peak 1) separated from the impurity peak(s) (FIG. 6A). The presence of the intended bsAb in the main peak was confirmed using the bridging ELISA assay (FIG. 6B) and LC/MS analysis (FIGS. 6C-6F). These data demonstrate that the bispecific antibodies with charge pairs can be purified on CEX-FPLC using stepwise elution methods.

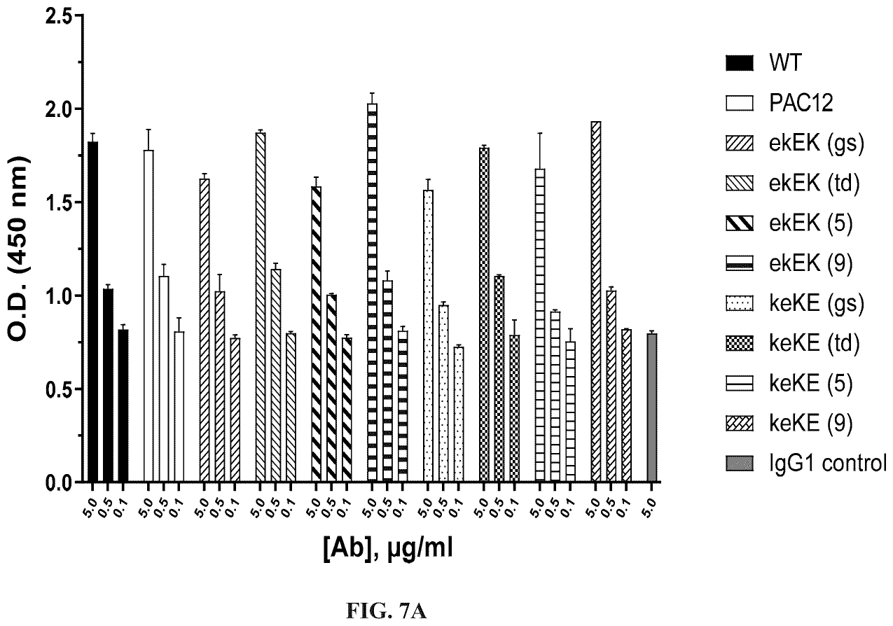

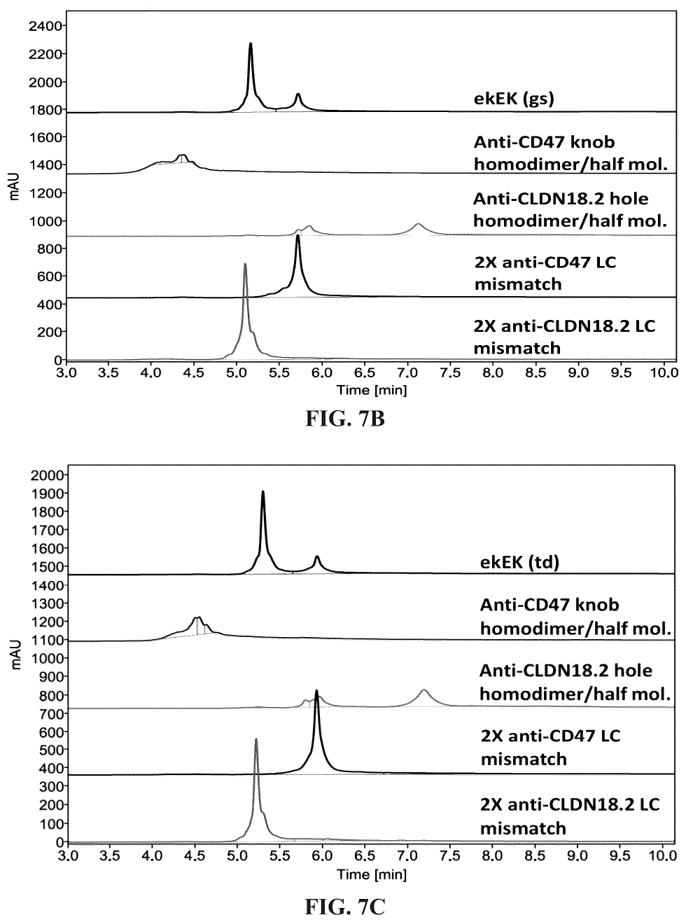

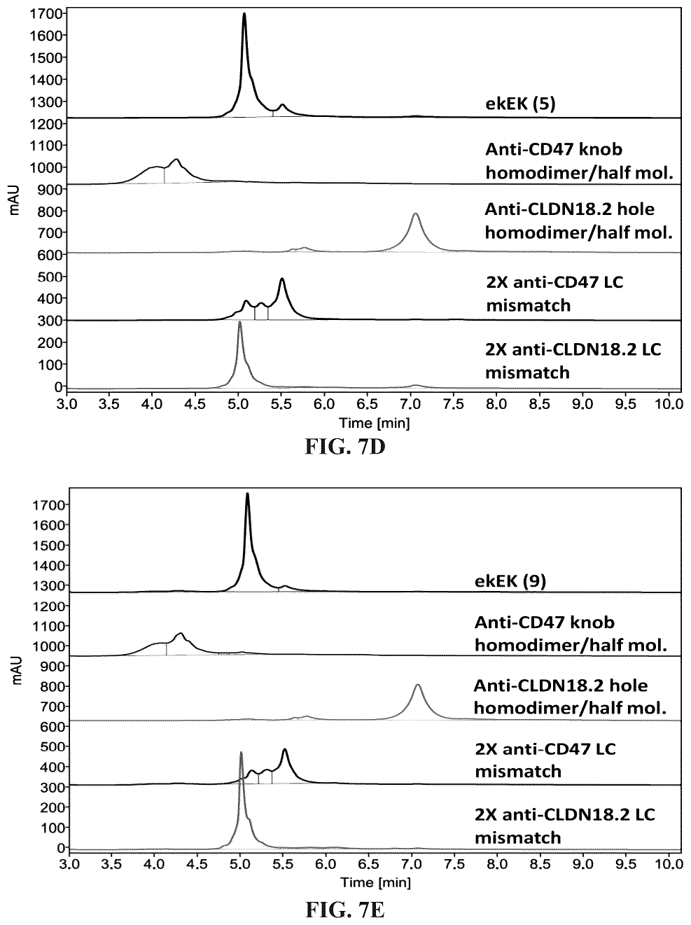

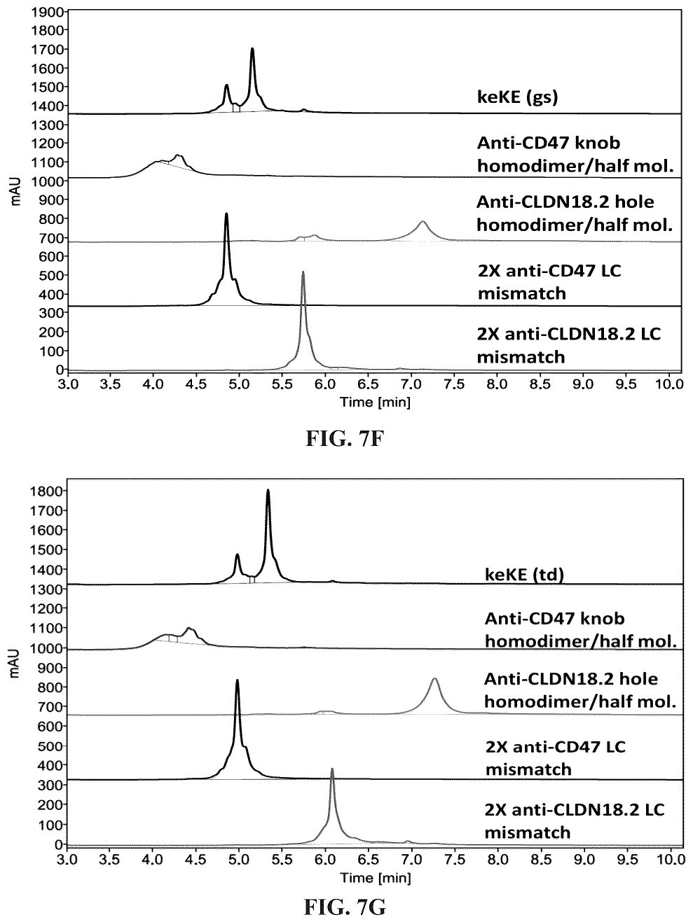

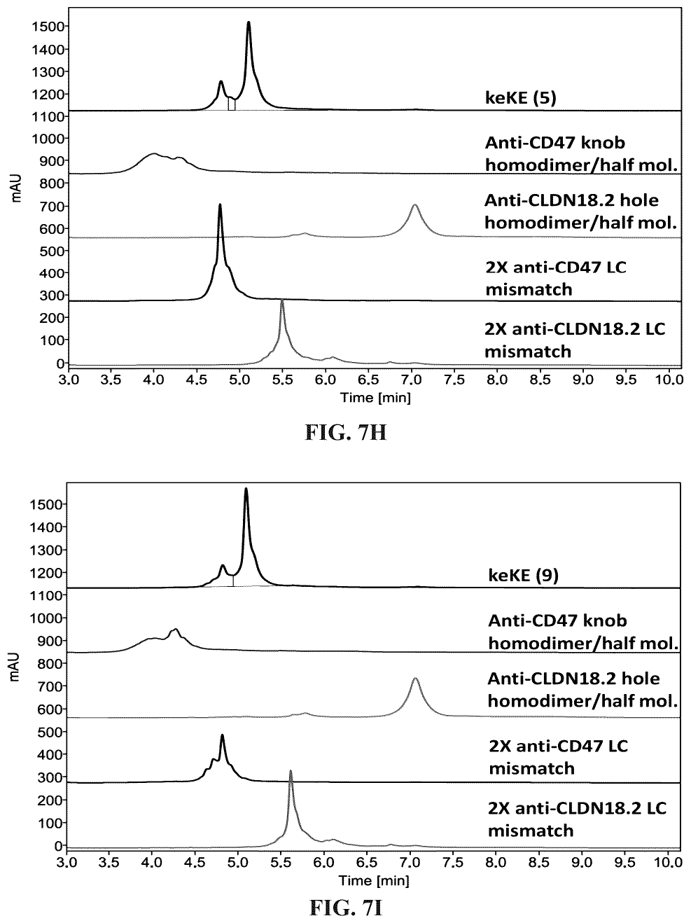

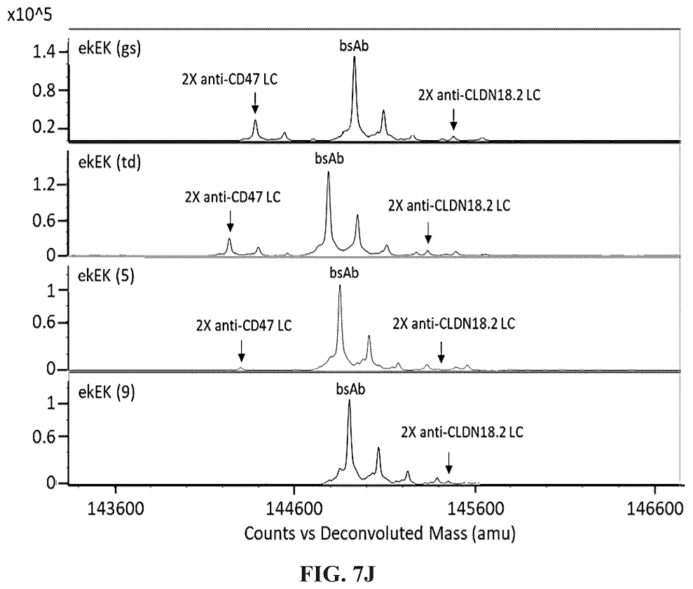

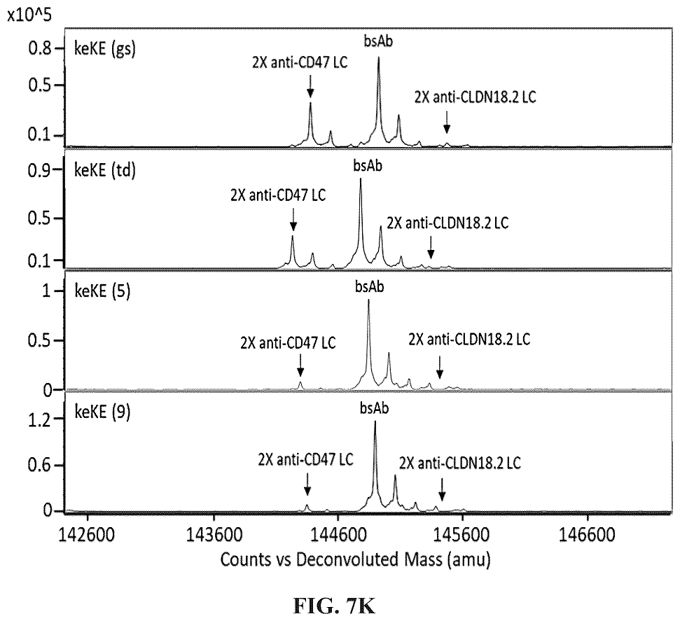

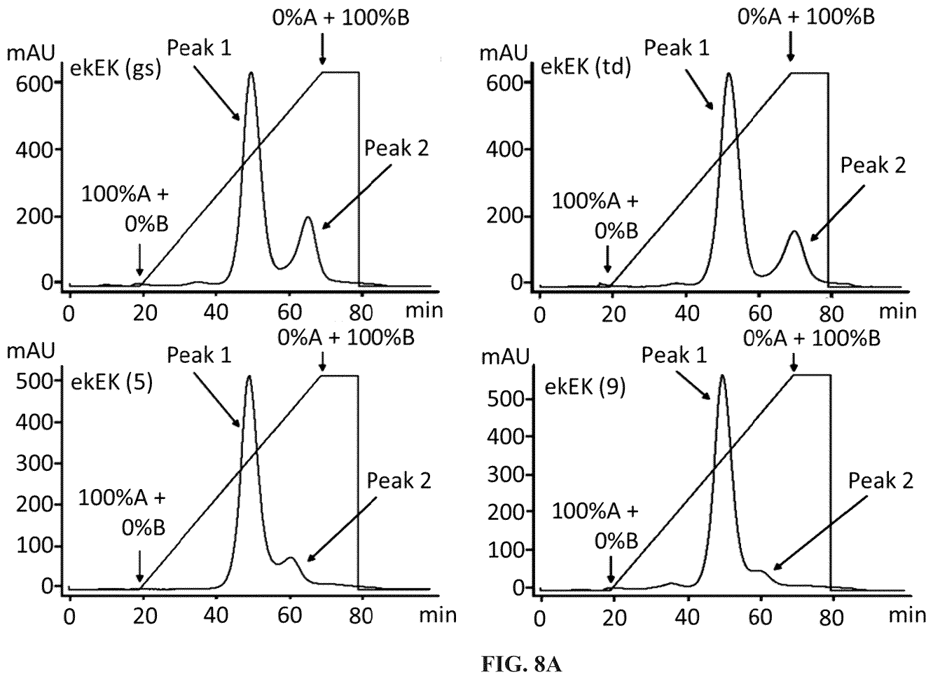

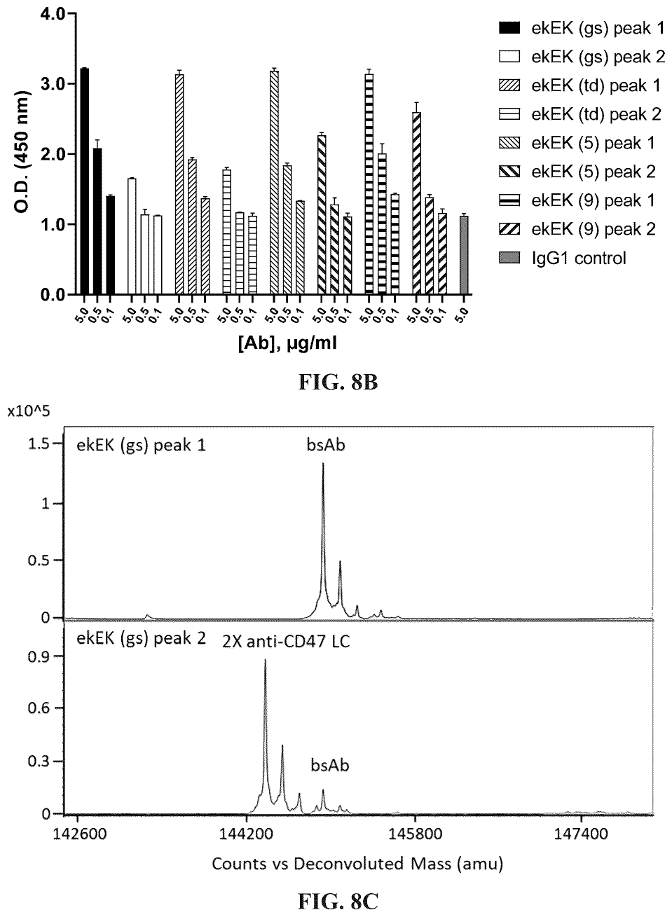

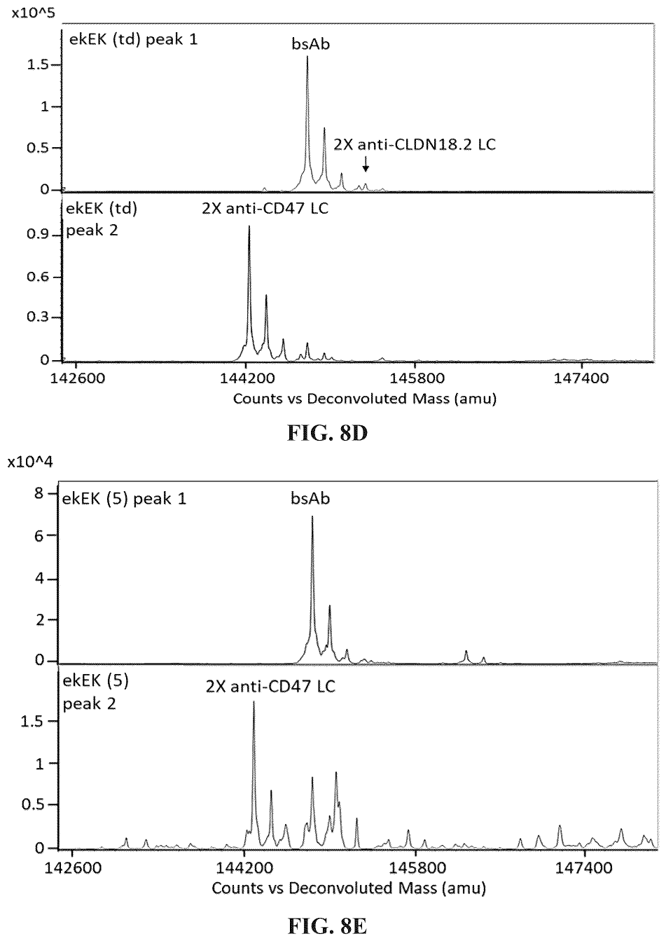

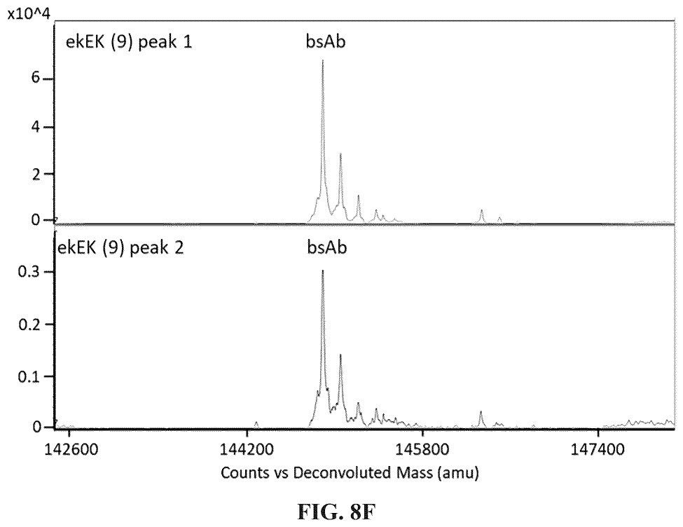

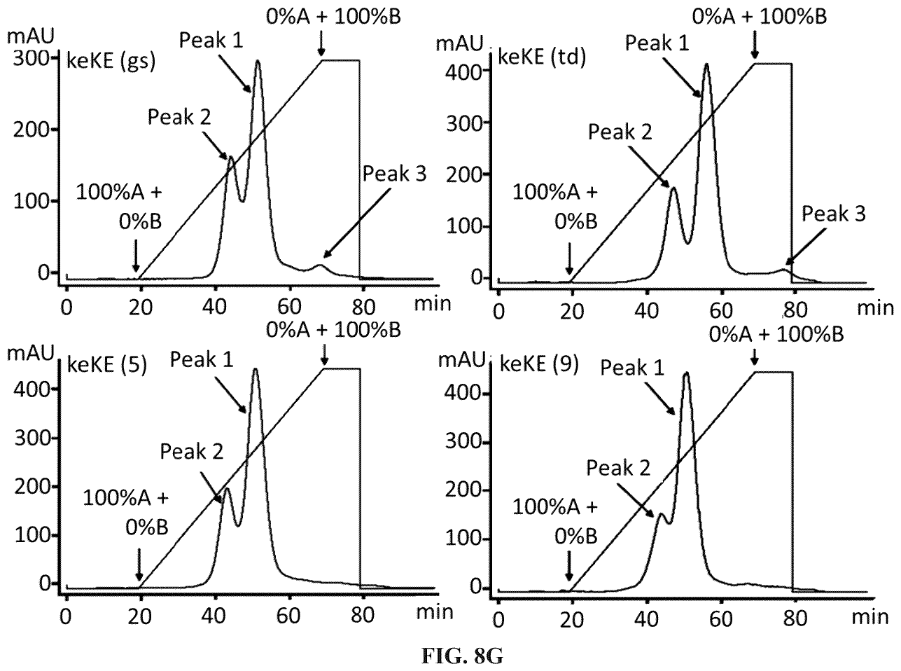

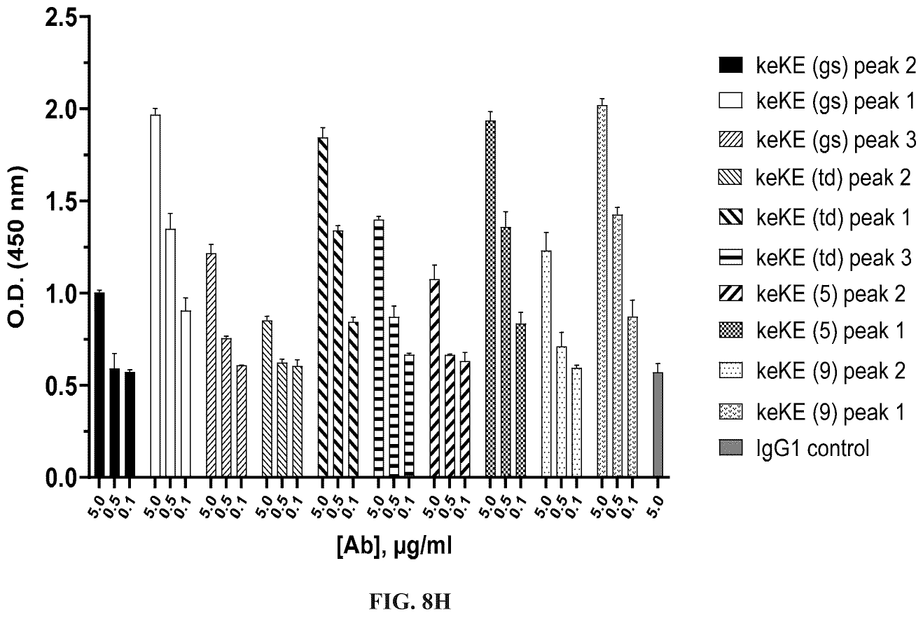

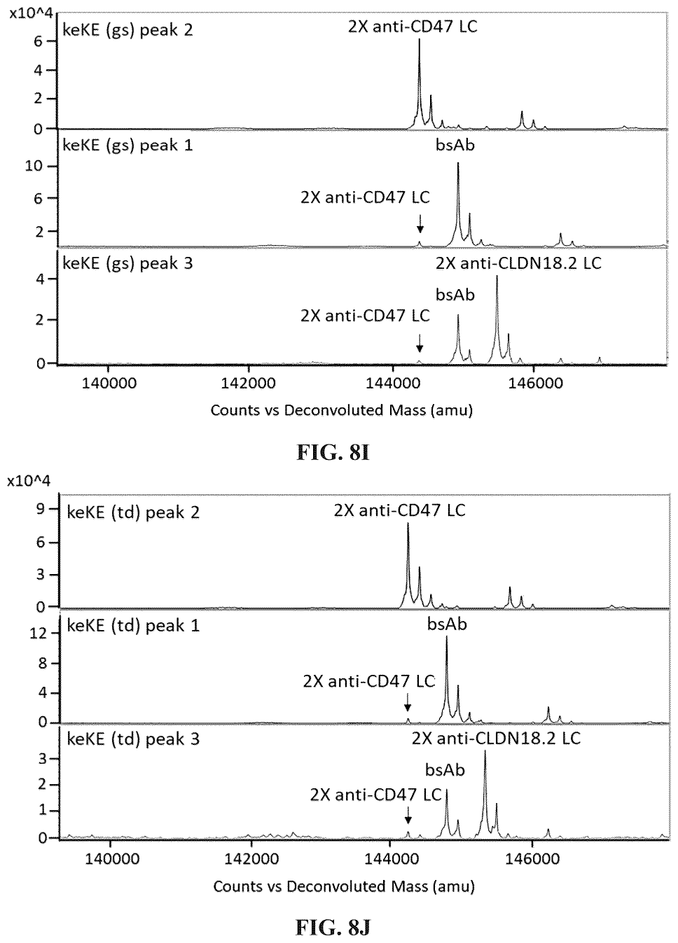

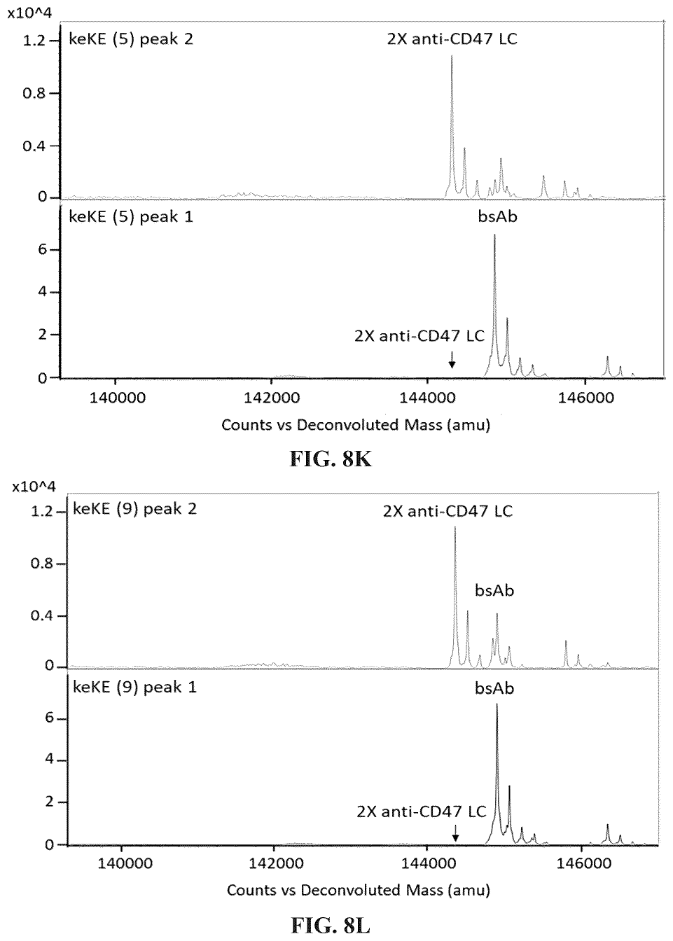

Bispecific antibody designs incorporating the “ekEK” or “keKE” charge pairs (Table 3) were produced in ExpiCHO-S cells transfected with both arm DNAs at the standard 1:1 DNA ratio. The Protein A purified samples from these designs were tested in the bridging ELISA assay to confirm bispecific activity (FIG. 7A). The same samples were also analyzed on CEX-HPLC using the linear salt gradient as described above along with their respective major impurity standards (FIGS. 7B-7I) and by LC/MS (FIGS. 7J-7K). The Protein A purified “ekEK” bsAb samples were separated on CEX-FPLC using the linear pH gradient [buffer A: 25 mM sodium phosphate (pH 5.8) and 50 mM NaCl; buffer B: 25 mM sodium phosphate (pH 8.0) and 50 mM NaCl] as described above. Compared with the WT, PAC12, ek and ke samples (FIG. 4E), the “ekEK” bsAb samples had better separation of the main peak (peak 1 where the bsAb is present) from one LC mismatch impurity species (FIG. 8A). The collected fractions from the peaks were analyzed to confirm the bsAb is in peak 1 using the same bridging ELISA assay (FIG. 8B) and LC/MS assay (FIGS. 8C-8F). Similarly, the “keKE” bsAb samples had better separation of the main peak (peak 1 where the bsAb is present) from one or two LC mismatch impurity species (FIG. 8G) when compared with the WT, PAC12, ek and ke samples (FIG. 4E). The collected fractions from the peaks were analyzed to confirm the bsAb is in peak 1 using the same bridging ELISA assay (FIG. 8H) and LC/MS assay (FIGS. 8I-8L). Bispecific antibodies “keKE (gs)” and “keKE (td)” were separated from both the 2×anti-CD47 LC and 2×CLDN18.2 LC mismatch species (FIGS. 8I-8J). Bispecific antibodies “keKE (5)” and “keKE (9)” were separated from the 2×anti-CD47 LC mismatch (FIG. 8K-8L); the 2×CLDN18.2 LC mismatch was not detected.

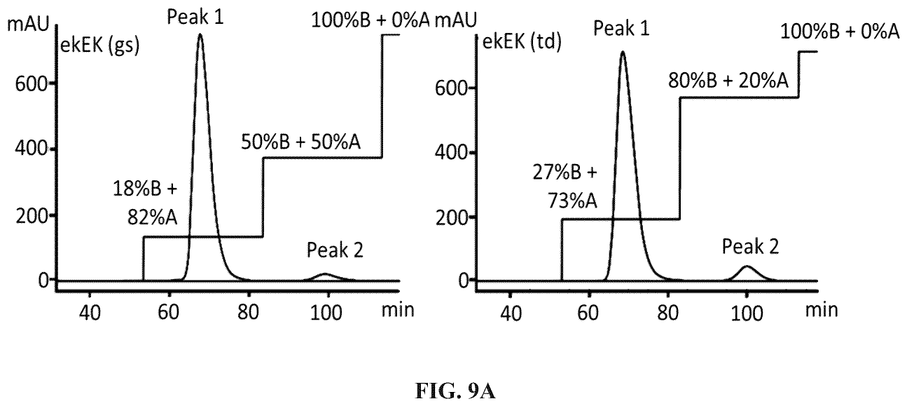

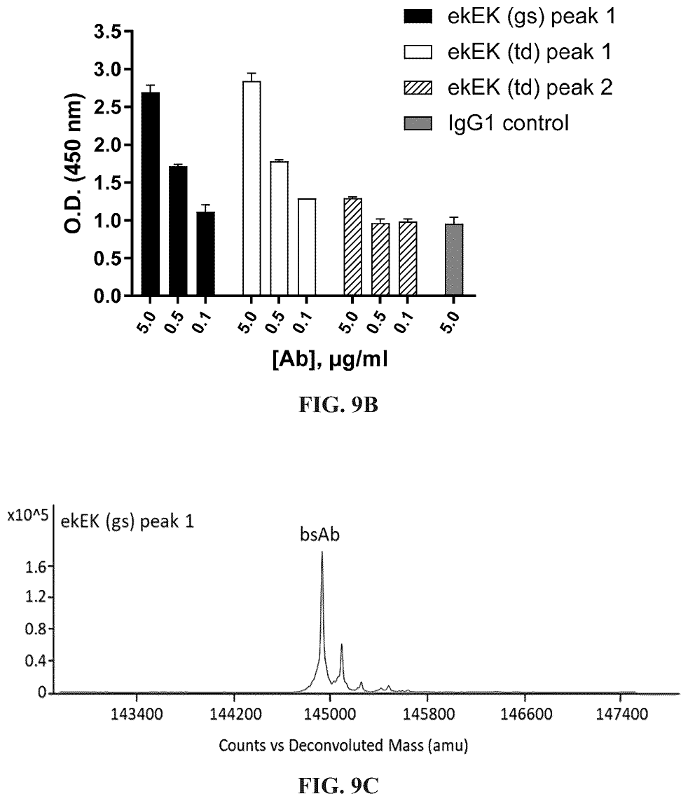

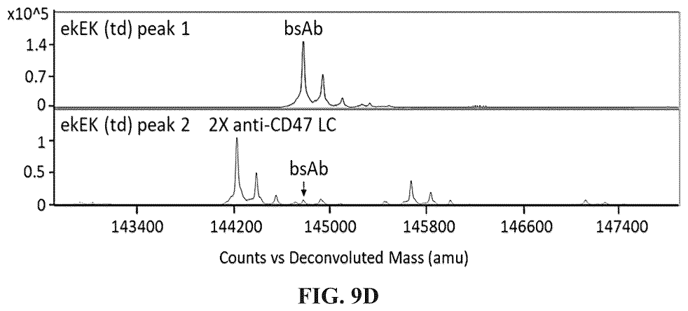

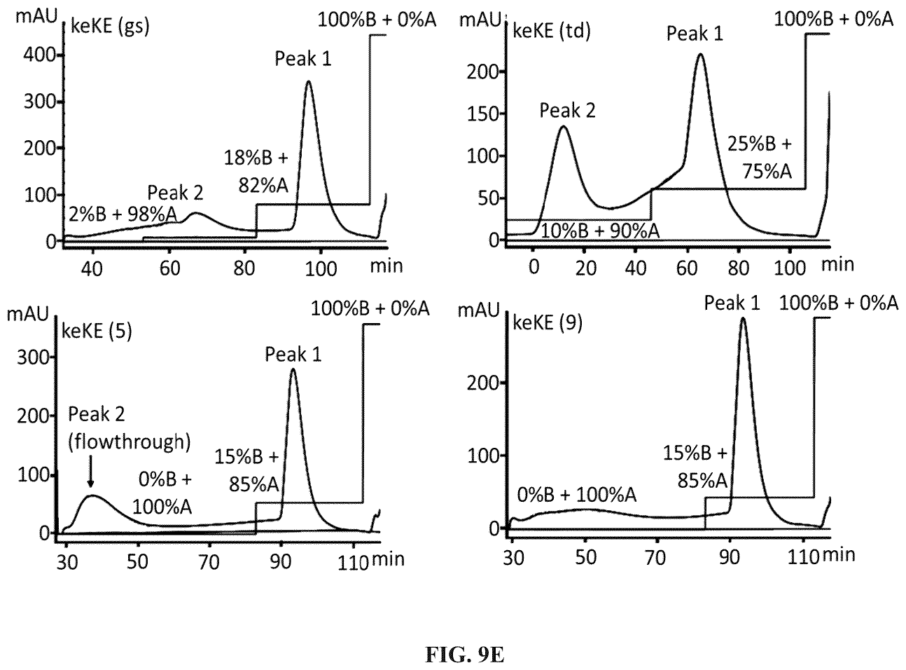

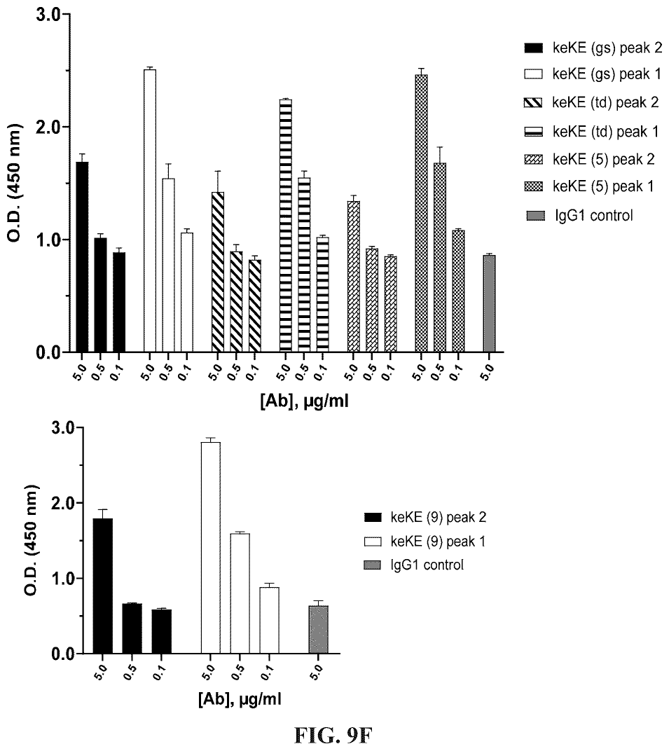

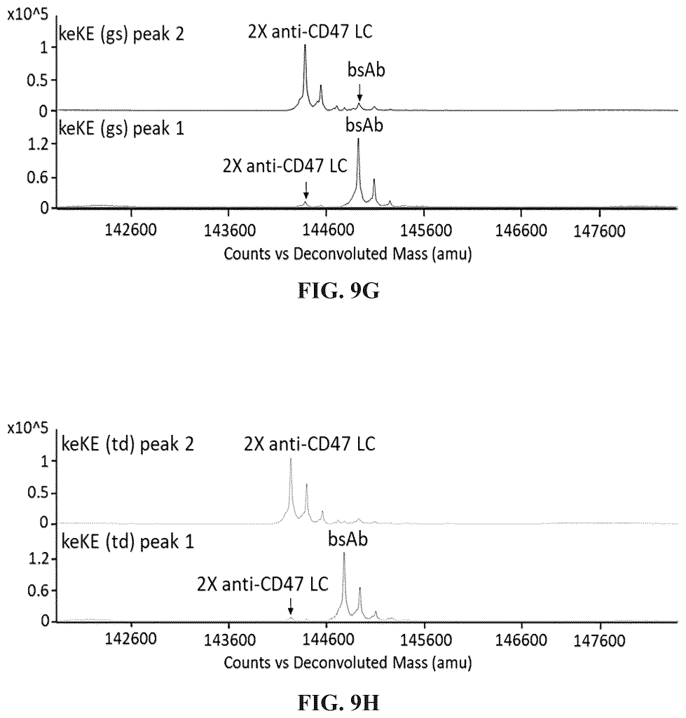

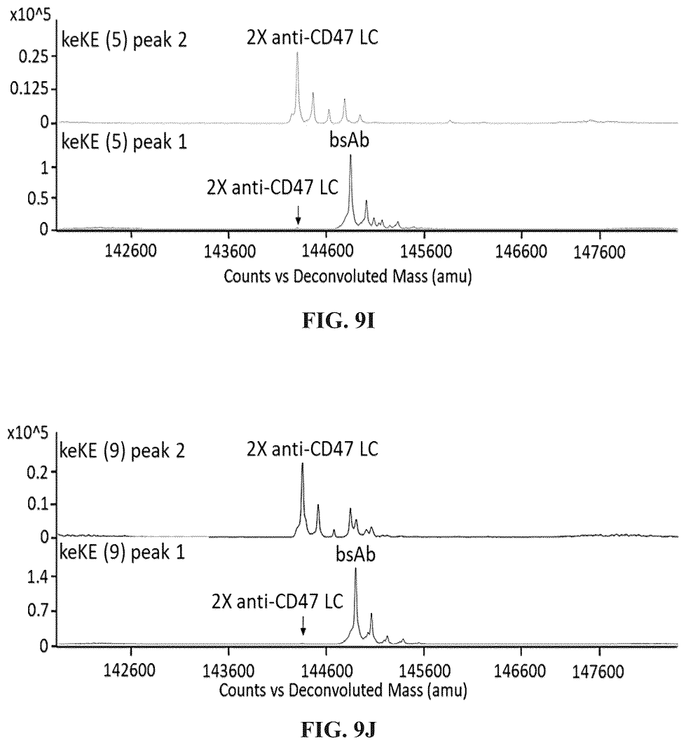

Two “ekEK” (FIG. 9A) and four “keKE” bispecific antibodies (FIG. 9E) were purified using customized stepwise elution methods [buffer A: 25 mM sodium phosphate (pH 5.8) and 80 mM NaCl; buffer B: 25 mM sodium phosphate (pH 8.0) and 40 mM NaCl] on CEX-FPLC and the activity of the purified bispecific antibodies were confirmed with the bridging ELISA assay (FIGS. 9B and 9F) and the LC/MS assay (FIGS. 9C-9D and 9G-9J). These results indicate that the charge pairs in Table 1 can be used by combining with other charge pairs (the ek or ke designs; Table 3) to facilitate the purification of bsAbs on CEX-FPLC. The sequences of various regions in the anti-CD47 antibody arm and the anti-CLDN18.2 antibody with different charge pair designs are listed in Table 6. These anti-CD47 antibody sequences can be used to construct bispecific antibodies with another antibody arm such as anti-CLDN18.2 to which the corresponding charge pairs can be introduced according to the designs in Table 3.

TABLE 6
Sequences of various regions of the anti-CD47 antibody arm and
the anti-CLDN18.2 arm with charge pairs

| | | SEQ |
|---|---|---|
| | | ID |
| Region name | Sequence | NO: |
| Anti-CD47 VH Q39E | QVQLVQSGAEVKKPGASVKVSCKASGYTFTSYWMHWVREAPGQGLE | 23 |
| | WIGNIDPSDSETHYAQKFQGRVTLTVDKSTSTVYMELSSLRSEDTA | |
| | VYYCAGTDLAYWGQGTLVTVSS | |
| Anti-CD47 VL Q38K | EIVLTQSPGTLSLSPGERATLSCHASQNINVWLSWYQKKPGQAPRL | 24 |
| | LIYKASNLHTGIPDRFSGSGSGTDFTLTISRLEPEDFAVYYCQQGQ | |
| | SYPFTFGQGTKVEIK | |
| Anti-CD47 VH Q39K | QVQLVQSGAEVKKPGASVKVSCKASGYTFTSYWMHWVRKAPGQGLE | 25 |
| | WIGNIDPSDSETHYAQKFQGRVTLTVDKSTSTVYMELSSLRSEDTA | |
| | VYYCAGTDLAYWGQGTLVTVSS | |
| Anti-CD47 VL Q38E | EIVLTQSPGTLSLSPGERATLSCHASQNINVWLSWYQEKPGQAPRL | 26 |
| | LIYKASNLHTGIPDRFSGSGSGTDFTLTISRLEPEDFAVYYCQQGQ | |
| | SYPFTFGQGTKVEIK | |
| Anti-CD47 CH1 | ASTKGPSVFPLAPSSCSTSGGTAALGCLVKDYFPEPVTVSWNSGAL | 27 |
| G166D | TSDVHTFPAVLQSSGLYSLSSVVTVPSSSLGTQTYICNVNHKPSNT | |
| | KVDKKVEPKSS | |
| Anti-CD47 CH1 | ASTKGPSVFPLAPSSCSTSGGTAALGCLVKDYFPEPVTVSWNSGAL | 28 |
| G166E | TSEVHTFPAVLQSSGLYSLSSVVTVPSSSLGTQTYICNVNHKPSNT | |
| | KVDKKVEPKSS | |
| Anti-CD47 CL S114K | RTVAAPKVFIFPPSDEQLKSGTASVVCLLNNFYPREAKVQWKVDNA | 29 |
| | LQSGNSQESVTEQDSKDSTYSLSSTLTLSKADYEKHKVYACEVTHQ | |
| | GLSSPVTKSCNRGES | |
| Anti-CD47 CL S114R | RTVAAPRVFIFPPSDEQLKSGTASVVCLLNNFYPREAKVQWKVDNA | 30 |
| | LQSGNSQESVTEQDSKDSTYSLSSTLTLSKADYEKHKVYACEVTHQ | |
| | GLSSPVTKSCNRGES | |
| Anti-CD47 CH1 | ASTKGPSVFPLAPSSCSTSGGTAALGCLVKDYFPEPVTVSWNSGAL | 31 |
| G166K | TSKVHTFPAVLQSSGLYSLSSVVTVPSSSLGTQTYICNVNHKPSNT | |
| | KVDKKVEPKSS | |
| Anti-CD47 CH1 | ASTKGPSVFPLAPSSCSTSGGTAALGCLVKDYFPEPVTVSWNSGAL | 32 |
| G166R | TSRVHTFPAVLQSSGLYSLSSVVTVPSSSLGTQTYICNVNHKPSNT | |
| | KVDKKVEPKSS | |
| Anti-CD47 CL S114D | RTVAAPDVFIFPPSDEQLKSGTASVVCLLNNFYPREAKVQWKVDNA | 33 |
| | LQSGNSQESVTEQDSKDSTYSLSSTLTLSKADYEKHKVYACEVTHQ | |
| | GLSSPVTKSCNRGES | |
| Anti-CD47 CL S114E | RTVAAPEVFIFPPSDEQLKSGTASVVCLLNNFYPREAKVQWKVDNA | 34 |
| | LQSGNSQESVTEQDSKDSTYSLSSTLTLSKADYEKHKVYACEVTHQ | |
| | GLSSPVTKSCNRGES | |
| Anti-CD47 CH1 | ASTKGPSVFPLAPSSCSTSGGTAALGCLVKDYFPEPVTVSWNSGAL | 35 |
| T187D | TSGVHTFPAVLQSSGLYSLSSVVDVPSSSLGTQTYICNVNHKPSNT | |
| | KVDKKVEPKSS | |
| Anti-CD47 CH1 | ASTKGPSVFPLAPSSCSTSGGTAALGCLVKDYFPEPVTVSWNSGAL | 36 |
| T187E | TSGVHTFPAVLQSSGLYSLSSVVEVPSSSLGTQTYICNVNHKPSNT | |
| | KVDKKVEPKSS | |
| Anti-CD47 CL D170K | RTVAAPSVFIFPPSDEQLKSGTASVVCLLNNFYPREAKVQWKVDNA | 37 |
| | LQSGNSQESVTEQDSKKSTYSLSSTLTLSKADYEKHKVYACEVTHQ | |
| | GLSSPVTKSCNRGES | |
| Anti-CD47 CL D170R | RTVAAPSVFIFPPSDEQLKSGTASVVCLLNNFYPREAKVQWKVDNA | 38 |
| | LQSGNSQESVTEQDSKRSTYSLSSTLTLSKADYEKHKVYACEVTHQ | |
| | GLSSPVTKSCNRGES | |
| Anti-CD47 CH1 | ASTKGPSVFPLAPSSCSTSGGTAALGCLVKDYFPEPVTVSWNSGAL | 39 |
| T187K | TSGVHTFPAVLQSSGLYSLSSVVKVPSSSLGTQTYICNVNHKPSNT | |
| | KVDKKVEPKSS | |
| Anti-CD47 CH1 | ASTKGPSVFPLAPSSCSTSGGTAALGCLVKDYFPEPVTVSWNSGAL | 40 |
| T187R | TSGVHTFPAVLQSSGLYSLSSVVRVPSSSLGTQTYICNVNHKPSNT | |
| | KVDKKVEPKSS | |
| Anti-CD47 CL D170D | RTVAAPSVFIFPPSDEQLKSGTASVVCLLNNFYPREAKVQWKVDNA | 41 |
| | LQSGNSQESVTEQDSKDSTYSLSSTLTLSKADYEKHKVYACEVTHQ | |
| | GLSSPVTKSCNRGES | |
| Anti-CD47 CL D170E | RTVAAPSVFIFPPSDEQLKSGTASVVCLLNNFYPREAKVQWKVDNA | 42 |
| | LQSGNSQESVTEQDSKESTYSLSSTLTLSKADYEKHKVYACEVTHQ | |
| | GLSSPVTKSCNRGES | |
| Anti-CD47 CH1 | ASTKGPSVFPLAPDSCSTSGGTAALGCLVKDYFPEPVTVSWNSGAL | 43 |
| S131D | TSGVHTFPAVLQSSGLYSLSSVVTVPSSSLGTQTYICNVNHKPSNT | |
| | KVDKKVEPKSS | |
| Anti-CD47 CH1 | ASTKGPSVFPLAPESCSTSGGTAALGCLVKDYFPEPVTVSWNSGAL | 44 |
| S131E | TSGVHTFPAVLQSSGLYSLSSVVTVPSSSLGTQTYICNVNHKPSNT | |
| | KVDKKVEPKSS | |
| Anti-CD47 CL P119K | RTVAAPSVFIFKPSDEQLKSGTASVVCLLNNFYPREAKVQWKVDNA | 45 |
| | LQSGNSQESVTEQDSKDSTYSLSSTLTLSKADYEKHKVYACEVTHQ | |
| | GLSSPVTKSCNRGES | |
| Anti-CD47 CL P119R | RTVAAPSVFIFRPSDEQLKSGTASVVCLLNNFYPREAKVQWKVDNA | 46 |
| | LQSGNSQESVTEQDSKDSTYSLSSTLTLSKADYEKHKVYACEVTHQ | |
| | GLSSPVTKSCNRGES | |
| Anti-CD47 CH1 | ASTKGPSVFPLAPKSCSTSGGTAALGCLVKDYFPEPVTVSWNSGAL | 47 |
| S131K | TSGVHTFPAVLQSSGLYSLSSVVTVPSSSLGTQTYICNVNHKPSNT | |
| | KVDKKVEPKSS | |
| Anti-CD47 CH1 | ASTKGPSVFPLAPRSCSTSGGTAALGCLVKDYFPEPVTVSWNSGAL | 48 |
| S131R | TSGVHTFPAVLQSSGLYSLSSVVTVPSSSLGTQTYICNVNHKPSNT | |
| | KVDKKVEPKSS | |
| Anti-CD47 CL P119D | RTVAAPSVFIFDPSDEQLKSGTASVVCLLNNFYPREAKVQWKVDNA | 49 |
| | LQSGNSQESVTEQDSKDSTYSLSSTLTLSKADYEKHKVYACEVTHQ | |
| | GLSSPVTKSCNRGES | |
| Anti-CD47 CL P119E | RTVAAPSVFIFEPSDEQLKSGTASVVCLLNNFYPREAKVQWKVDNA | 50 |
| | LQSGNSQESVTEQDSKDSTYSLSSTLTLSKADYEKHKVYACEVTHQ | |
| | GLSSPVTKSCNRGES | |
| Anti-CD47 CH1 | ASTKGPSVFPLDPSSCSTSGGTAALGCLVKDYFPEPVTVSWNSGAL | 51 |
| A129D | TSGVHTFPAVLQSSGLYSLSSVVTVPSSSLGTQTYICNVNHKPSNT | |
| | KVDKKVEPKSS | |
| Anti-CD47 CH1 | ASTKGPSVFPLEPSSCSTSGGTAALGCLVKDYFPEPVTVSWNSGAL | 52 |
| A129E | TSGVHTFPAVLQSSGLYSLSSVVTVPSSSLGTQTYICNVNHKPSNT | |
| | KVDKKVEPKSS | |
| Anti-CD47 CL S121K | RTVAAPSVFIFPPKDEQLKSGTASVVCLLNNFYPREAKVQWKVDNA | 53 |
| | LQSGNSQESVTEQDSKDSTYSLSSTLTLSKADYEKHKVYACEVTHQ | |
| | GLSSPVTKSCNRGES | |
| Anti-CD47 CL S121R | RTVAAPSVFIFPPRDEQLKSGTASVVCLLNNFYPREAKVQWKVDNA | 54 |
| | LQSGNSQESVTEQDSKDSTYSLSSTLTLSKADYEKHKVYACEVTHQ | |
| | GLSSPVTKSCNRGES | |
| Anti-CD47 CH1 | ASTKGPSVFPLKPSSCSTSGGTAALGCLVKDYFPEPVTVSWNSGAL | 55 |
| A129K | TSGVHTFPAVLQSSGLYSLSSVVTVPSSSLGTQTYICNVNHKPSNT | |
| | KVDKKVEPKSS | |
| Anti-CD47 CH1 | ASTKGPSVFPLRPSSCSTSGGTAALGCLVKDYFPEPVTVSWNSGAL | 56 |
| A129R | TSGVHTFPAVLQSSGLYSLSSVVTVPSSSLGTQTYICNVNHKPSNT | |
| | KVDKKVEPKSS | |
| Anti-CD47 CL S121D | RTVAAPSVFIFPPDDEQLKSGTASVVCLLNNFYPREAKVQWKVDNA | 57 |
| | LQSGNSQESVTEQDSKDSTYSLSSTLTLSKADYEKHKVYACEVTHQ | |
| | GLSSPVTKSCNRGES | |
| Anti-CD47 CL S121E | RTVAAPSVFIFPPEDEQLKSGTASVVCLLNNFYPREAKVQWKVDNA | 58 |
| | LQSGNSQESVTEQDSKDSTYSLSSTLTLSKADYEKHKVYACEVTHQ | |
| | GLSSPVTKSCNRGES | |
| Anti-CLDN18.2 VH | EVOLVESGGGLVQPGGSLRLSCAASGFIFSSFGMHWVRKAPGKGLE | 59 |
| Q39K | WVAYISSGRSTMYYADSVKGRFTISRDNSKNTLYLQMNSLTAEDTA | |
| | VYYCARGGFYGNSLDYWGQGTLVTVSS | |
| Anti- CLDN18.2 VL | DIQMTQSPSSLSASVGDRVTITCKSSLSLLNSGNQKNYLTWYQEKP | 60 |
| Q38E | GKAPKLLIYWASTRESGVPSRFSGSGSGTDFTLTISSLQPEDFATY | |
| | SCQNAYSYPLTFGQGTKVEIK | |
| Anti-CLDN18.2 VH | EVQLVESGGGLVQPGGSLRLSCAASGFIFSSFGMHWVREAPGKGLE | 61 |
| Q39E | WVAYISSGRSTMYYADSVKGRFTISRDNSKNTLYLQMNSLTAEDTA | |
| | VYYCARGGFYGNSLDYWGQGTLVTVSS | |
| Anti-CLDN18.2 VL | DIQMTQSPSSLSASVGDRVTITCKSSLSLLNSGNQKNYLTWYQKKP | 62 |
| Q38K | GKAPKLLIYWASTRESGVPSRFSGSGSGTDFTLTISSLQPEDFATY | |
| | SCQNAYSYPLTFGQGTKVEIK | |
| Anti-CLDN18.2 CH1 | ASTKGPSVFPLAPSSKSTSGGTAALGCLVKDYFPEPVTVSWNSGAL | 63 |
| G166K | TSKVHTFPAVLQSSGLYSLSSVVTVPSSSLGTQTYICNVNHKPSNT | |
| | KVDKKVEPKSC | |
| Anti-CLDN18.2 CL | RTVAAPEVFIFPPSDEQLKSGTASVVCLLNNFYPREAKVQWKVDNA | 64 |
| S114E | LQSGNSQESVTEQDSKDSTYSLSSTLTLSKADYEKHKVYACEVTHQ | |
| | GLSSPVTKSFNRGEC | |
| Anti-CLDN18.2 CH1 | ASTKGPSVFPLAPSSKSTSGGTAALGCLVKDYFPEPVTVSWNSGAL | 65 |
| G166E | TSEVHTFPAVLQSSGLYSLSSVVTVPSSSLGTQTYICNVNHKPSNT | |
| | KVDKKVEPKSC | |
| Anti-CLDN18.2 CL | RTVAAPKVFIFPPSDEQLKSGTASVVCLLNNFYPREAKVQWKVDNA | 66 |
| S114K | LQSGNSQESVTEQDSKDSTYSLSSTLTLSKADYEKHKVYACEVTHQ | |
| | GLSSPVTKSFNRGEC | |
| Anti-CLDN18.2 CH1 | ASTKGPSVFPLAPSSKSTSGGTAALGCLVKDYFPEPVTVSWNSGAL | 67 |
| T187K | TSGVHTFPAVLQSSGLYSLSSVVKVPSSSLGTQTYICNVNHKPSNT | |
| | KVDKKVEPKSC | |
| Anti-CLDN18.2 CL | RTVAAPSVFIFPPSDEQLKSGTASVVCLLNNFYPREAKVQWKVDNA | 68 |
| D170E | LQSGNSQESVTEQDSKESTYSLSSTLTLSKADYEKHKVYACEVTHQ | |
| | GLSSPVTKSFNRGEC | |
| Anti-CLDN18.2 CH1 | ASTKGPSVFPLAPSSKSTSGGTAALGCLVKDYFPEPVTVSWNSGAL | 69 |
| T187E | TSGVHTFPAVLQSSGLYSLSSVVEVPSSSLGTQTYICNVNHKPSNT | |
| | KVDKKVEPKSC | |
| Anti-CLDN18.2 CL | RTVAAPSVFIFPPSDEQLKSGTASVVCLLNNFYPREAKVQWKVDNA | 70 |
| D170K | LQSGNSQESVTEQDSKKSTYSLSSTLTLSKADYEKHKVYACEVTHQ | |
| | GLSSPVTKSFNRGEC | |
| Anti-CLDN18.2 CH1 | ASTKGPSVFPLAPKSKSTSGGTAALGCLVKDYFPEPVTVSWNSGAL | 71 |
| S131K | TSGVHTFPAVLQSSGLYSLSSVVTVPSSSLGTQTYICNVNHKPSNT | |
| | KVDKKVEPKSC | |
| Anti-CLDN18.2 CL | RTVAAPSVFIFEPSDEQLKSGTASVVCLLNNFYPREAKVQWKVDNA | 72 |
| P119E | LQSGNSQESVTEQDSKDSTYSLSSTLTLSKADYEKHKVYACEVTHQ | |
| | GLSSPVTKSFNRGEC | |
| Anti-CLDN18.2 CH1 | ASTKGPSVFPLKPSSKSTSGGTAALGCLVKDYFPEPVTVSWNSGAL | 73 |
| A129K | TSGVHTFPAVLQSSGLYSLSSVVTVPSSSLGTQTYICNVNHKPSNT | |
| | KVDKKVEPKSC | |
| Anti-CLDN18.2 CL | RTVAAPSVFIFPPEDEQLKSGTASVVCLLNNFYPREAKVQWKVDNA | 74 |
| S121E | LQSGNSQESVTEQDSKDSTYSLSSTLTLSKADYEKHKVYACEVTHQ | |
| | GLSSPVTKSFNRGEC | |
| Anti-CLDN18.2 CH1 | ASTKGPSVFPLAPESKSTSGGTAALGCLVKDYFPEPVTVSWNSGAL | 75 |
| S131E | TSGVHTFPAVLQSSGLYSLSSVVTVPSSSLGTQTYICNVNHKPSNT | |
| | KVDKKVEPKSC | |
| Anti-CLDN18.2 CL | RTVAAPSVFIFKPSDEQLKSGTASVVCLLNNFYPREAKVQWKVDNA | 76 |
| P119K | LQSGNSQESVTEQDSKDSTYSLSSTLTLSKADYEKHKVYACEVTHQ | |
| | GLSSPVTKSFNRGEC | |
| Anti-CLDN18.2 CH1 | ASTKGPSVFPLEPSSKSTSGGTAALGCLVKDYFPEPVTVSWNSGAL | 77 |
| A129E | TSGVHTFPAVLQSSGLYSLSSVVTVPSSSLGTQTYICNVNHKPSNT | |
| | KVDKKVEPKSC | |
| Anti-CLDN18.2 CL | RTVAAPSVFIFPPKDEQLKSGTASVVCLLNNFYPREAKVQWKVDNA | 78 |
| S121K | LQSGNSQESVTEQDSKDSTYSLSSTLTLSKADYEKHKVYACEVTHQ | |
| | GLSSPVTKSFNRGEC | |

Note:
The residues resulting from amino acid substitutions are in bold and underlined form.

### Example 3: Properties of the Anti-CD3/Anti-DLL3 Bispecific Antibodies With Charge Pairs

The Protein A purified samples of the anti-CD3/anti-DLL3 bsAb were pH adjusted to a final pH of 5.5 and loaded directly onto a poros XS (Thermo) CEX column pre-equilibrated with 25 mM sodium phosphate (pH 5.8)+210 mM NaCl. Samples were eluted with a linear gradient [Buffer A−25 mM sodium phosphate (pH 5.8)+210 mM NaCl; Buffer B−25 mM sodium phosphate (pH 8)+115 mM NaCl]. Eluted fractions were analyzed by strong cation exchange (SCX) HPLC, and fractions showing the complete elimination of 2×anti-DLL3 LC mismatch (the HC heterodimer with the anti-DLL3 LC matched on both arms) were pooled. (NH₄)₂SO₄ was added to pooled fractions to a final concentration of 700 mM, and the sample was loaded directly onto a Butyl Sepharose High Performance (Cytiva) HIC (hydrophobic interaction chromatography) column pre-equilibrated with 50 mM tris (pH 7.5)+700 mM (NH₄)₂SO₄+3% glycerol. Samples were eluted using a linear gradient [Buffer A−50 mM tris (pH 7.5) +700 mM (N 14)25O4 +3% glycerol; Buffer B−50 mM tris (pH 7.5)+10% glycerol]. Eluted fractions were analyzed by HIC HPLC, and fractions showing the complete elimination of 2×anti-CD3 LC mismatch (the HC heterodimer with the anti-DLL3 LC matched on both arms) were pooled as purified protein.

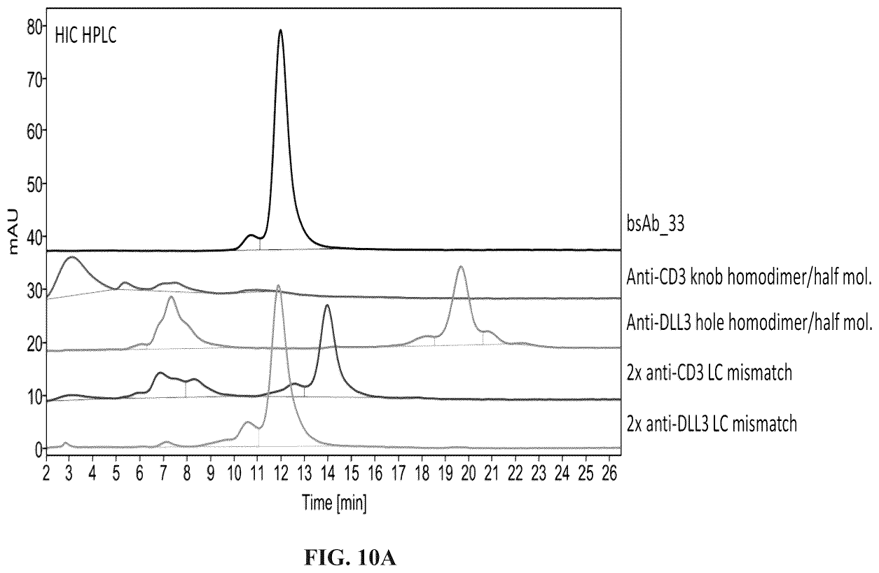

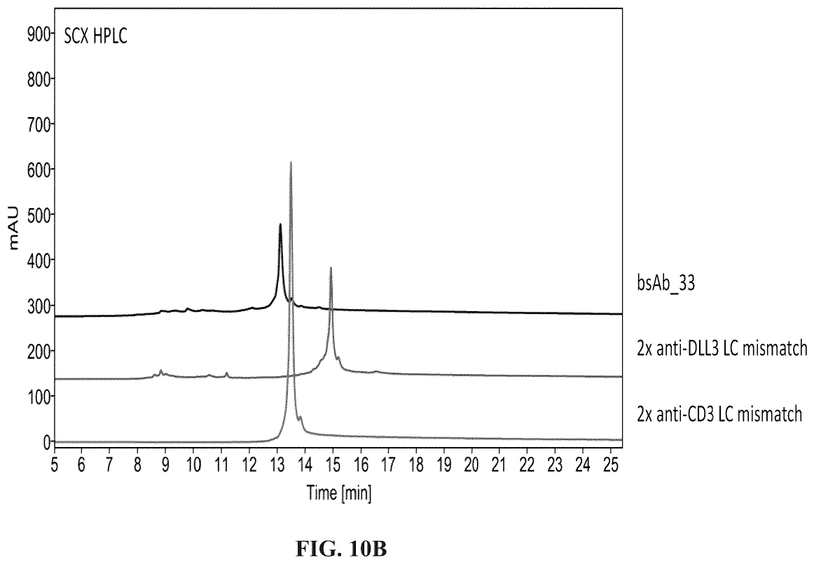

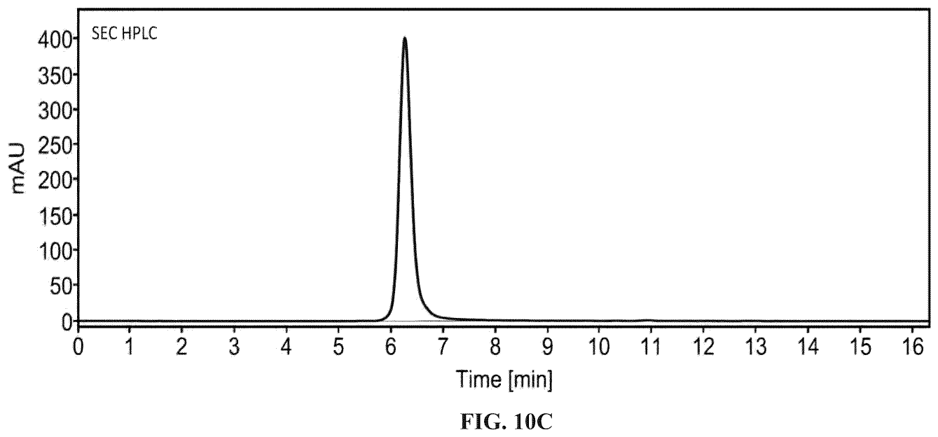

The purified bispecific antibodies were analyzed with HIC HPLC, SCX HPLC and SEC HPLC (FIGS. 10A-10C). The standards for the potential impurities were generated using transient transfection with only the components for the given impurity (i.e., to generate the 2×anti-CD3 LC mismatch, only the anti-DLL3 HC, anti-CD3 HC and anti-CD3 LC were used in the transfection to force the formation of the anti-CD3 LC mismatch standard; a homodimer/half molecule was generated by transfecting cells with the HC and LC of the given arm only), purified with Protein A and used as references in the analyses (FIGS. 10A-10B). The data indicate that the purified bsAb_33 has high purity.

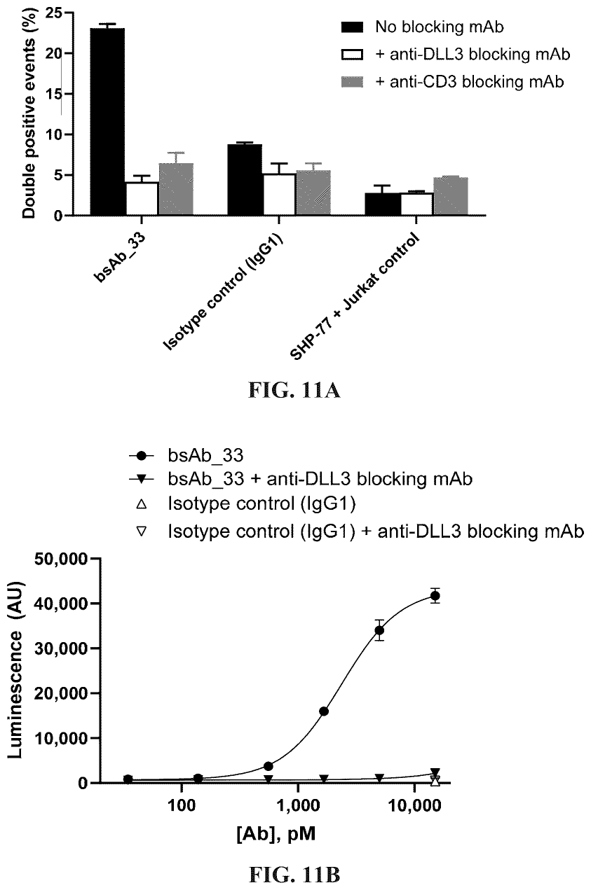

To assess the binding activity of bsAb_33 to both DLL3 and CD3 at the same time, the purified bsAb was incubated with SHP-77 cells and Jurkat cells which were labeled with different fluorescent markers. The double-stained event induced by bsAb_33 was detected and quantified by flow cytometry. Briefly, Jurkat cells were stained with Violet Proliferation Dye 450 (BD, Cat: 562158) and SHP-77 cells were stained by CFSE (ThermoFisher, Cat: 34554) according to the manufacturer's protocol. The labelled SHP-77 and Jurkat cells at 1:1 ratio were then incubated with 2 μg/ml bsAb_33. For mAb blocking, SHP-77 cells were treated with 4.5 μM anti-DLL3 blocking mAb for 10 minutes at room temperature before incubation with Jurkat cells at the final concentration of 1.5 μM anti-DLL3 blocking mAb, or Jurkat cells were treated with 4.5 μM anti-CD3 blocking mAb for 10 minutes at room temperature before incubation with SHP-77 cells at the final concentration of 1.5 μM anti-CD3 blocking mAb. Following incubation in CO₂ incubator at 37° C. for 1 hour, the cells were ixed with 2% formaldehyde, washed once with TBS, resuspended in FACS buffer (HBSS, 0.1% BSA, 0.05% sodium azide) and then analyzed by flow cytometry (Attune NxT). The cross-linking of the two cell types in the presence of bsAb_33 was detected on FACS and the signal was inhibited by the anti-DLL3 or the anti-CD3 blocking mAb (FIG. 11A). These data demonstrate that bsAb_33 functions as a bsAb.

The bsAb bsAb_33 was also used to activate T cells in a functional T cell activation assay. A Jurkat NFAT Luciferase reporter cell line (BPS Bioscience) which conditionally expresses firefly luciferase upon activation including CD3 mediated activation was used. The reporter cells were incubated with SHP-77 target cells in the presence of bsAb_33 and with or without the presence of anti-DLL3 blocking antibody for 22 hours in growth media at 37° C. in a CO₂ incubator. The cells were then assayed for activation by a luciferase detection reagent and luminometer. The bsAb bsAb_33 induced dose-dependent activation of the reporter cells when incubated with the target cell SHP-77, and the signal was inhibited by the anti-DLL3 blocking antibody (the mAb version of the anti-DLL3 arm) (FIG. 11B), demonstrating the T cell engager function of bsAb_33.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. An isolated bispecific antibody or antigen-binding fragment thereof comprising:
a. a first heavy chain, H1;
b. a second heavy chain, H2;
c. a first light chain, L1; and
d. a second light chain, L2;
wherein H1 and L1 form a first arm comprising a first antigen-binding domain that specifically binds a first antigen, and
wherein H2 and L2 form a second arm comprising a second antigen-binding domain that specifically binds a second antigen, wherein
(a) H1 and H2 each comprises a CH1 region of human IgG1, IgG2, IgG3, or IgG4; and
(b) L1 and L2 each comprises a CL region of a human kappa light chain or a human lambda light chain;
wherein H1L1 and H2L2 each comprise a charge pair selected from the group consisting of the following amino acid substitutions:
(1) G166D/E in CH1 of H1 and S114K/R in CL of L1, respectively, and G166K/R in CH1 of H2 and S114D/E in CL of L2, respectively;
(2) T187D/E in CH1 of H1 and D/N170K/R in CL of L1, respectively, and T187K/R in CH1 of H2 and D/N170D/E in CL of L2, respectively;
(3) S131D/E in CH1 of H1 and P119K/R in CL of L1, respectively, and S131K/R in CH1 of H2 and P119D/E in CL of L2, respectively;
(4) A129D/E in CH1 of H1 and S121K/R in CL of L1, respectively, and A129K/R in CH1 of H2 and S121D/E in CL of L2, respectively;
(5) G166D/E in CH1 of H2 and S114K/R in CL of L2, respectively, and G166K/R in CH1 of H1 and S114D/E in CL of L1, respectively;
(6) T187D/E in CH1 of H2 and D/N170K/R in CL of L2, respectively, and T187K/R in CH1 of H1 and D/N170D/E in CL of L1, respectively;
(7) S131D/E in CH1 of H2 and P119K/R in CL of L2, respectively, and S131K/R in CH1 of H1 and P119D/E in CL of L1, respectively; or (8) A129D/E in CH1 of H2 and S121K/R in CL of L2, respectively, and A129K/R in CH1 of H1 and S121D/E in CL of L1, respectively.

2. The isolated bispecific antibody or antigen-binding fragment thereof of claim 1, wherein
(a) the two heavy chains H1 and H2 each comprise a VH region, a CH1 region, and a Fc region (containing CH2 and CH3 regions), wherein the VH regions have different amino acid sequences;
(b) the two heavy chains H1 and H2 each comprise a VH region, a CH1 region, and a Fc region (containing CH2 and CH3 regions), wherein the CH1 regions have different amino acid sequences;
(c) the two heavy chains H1 and H2 each comprise a VH region, a CH1 region, and a Fc region (containing CH2 and CH3 regions), wherein the Fc regions have different amino acid sequences;
(d) the two light chains L1 and L2 each comprise a VL region and a CL region, wherein the VL regions have different amino acid sequences; and/or
(e) the two light chains L1 and L2 each comprise a VL region and a CL region, wherein the CL regions have different amino acid sequences.

3. The isolated bispecific antibody or antigen-binding fragment thereof of claim 2, wherein H1 and H2 form a heterodimer.

4. The isolated bispecific antibody or antigen-binding fragment thereof of claim 1, wherein the isolated bispecific antibody comprises:
(a) a negatively charged amino acid (D or E) at G166, T187, S131, or A129 in CH1 of H1, the VH region of H1 and the VL region of L1 have a Q39E and a Q38K substitution mutation, respectively, and the VH region of H2 and the VL region of L2 have a Q39K and a Q38E substitution mutation, respectively; or
(b) a positively charged amino acid (K or R) at G166, T187, S131, or A129 in CH1 of H1, the VH region of H1 and the VL region of L1 have a Q39K and a Q38E substitution mutation, respectively, and the VH region of H2 and the VL region of L2 have a Q39E and a Q38K substitution mutation, respectively.

5. The isolated bispecific antibody or antigen-binding fragment thereof of claim 1,
wherein the CH1 and CL regions of one of the two arms comprise amino acid substitutions at an amino acid residue corresponding to the amino acid position of SEQ ID NO:17, 18, 19, or 20 for CH1 and SEQ ID NO:21 or 22 for CL;
wherein the amino acid substitutions in the CH1 and CL regions are selected from:
(1) K133C and C220X in CH1, and F209C and C214X in CL;
(2) R133C and C131X in CH1, and F209C and C214X in CL;
(3) R133C and C131X in CH1, and V209C and C214X in CL; or
(4) K133C and C220X in CH1, and V209C and C214X in CL;
wherein X is selected from S, A or G

6. The isolated bispecific antibody or antigen-binding fragment thereof of claim 1, wherein the first antigen-binding domain specifically binds CD47, and the second antigen-binding domain specifically binds a TAA expressed on the same cell as CD47.

7. The isolated bispecific antibody or antigen-binding fragment thereof of claim 6, wherein the second antigen-binding domain specifically binds CLDN18.2 expressed on the same cell as CD47.

8. The isolated bispecific antibody or antigen-binding fragment thereof of claim 6, wherein the anti-CD47 antigen-binding domain comprises the VH, CH1, VL, and CL comprising the amino acid sequences of:
(1) SEQ ID NOs: 1, 27, 2 and 29, respectively;
(2) SEQ ID NOs: 1, 28, 2 and 29, respectively;
(3) SEQ ID NOs: 1, 27, 2 and 30, respectively;
(4) SEQ ID NOs: 1, 28, 2 and 30, respectively;
(5) SEQ ID NOs: 1, 31, 2 and 33, respectively;
(6) SEQ ID NOs: 1, 32, 2 and 33, respectively;
(7) SEQ ID NOs: 1, 31, 2 and 34, respectively;
(8) SEQ ID NOs: 1, 32, 2 and 34, respectively;
(9) SEQ ID NOs: 1, 35, 2 and 37, respectively;
(10) SEQ ID NOs: 1, 36, 2 and 37, respectively;
(11) SEQ ID NOs: 1, 35, 2 and 38, respectively;
(12) SEQ ID NOs: 1, 36, 2 and 38, respectively;
(13) SEQ ID NOs: 1, 39, 2 and 41, respectively;
(14) SEQ ID NOs: 1, 40, 2 and 41, respectively;
(15) SEQ ID NOs: 1, 39, 2 and 42, respectively;
(16) SEQ ID NOs: 1, 40, 2 and 42, respectively;
(17) SEQ ID NOs: 1, 43, 2 and 45, respectively;
(18) SEQ ID NOs: 1, 44, 2 and 45, respectively;
(19) SEQ ID NOs: 1, 43, 2 and 46 respectively;
(20) SEQ ID NOs: 1, 44, 2 and 46, respectively;
(21) SEQ ID NOs: 1, 47, 2 and 49, respectively;
(22) SEQ ID NOs: 1, 48, 2 and 49, respectively;
(23) SEQ ID NOs: 1, 47, 2 and 50, respectively;
(24) SEQ ID NOs: 1, 48, 2 and 50, respectively;
(25) SEQ ID NOs: 1, 51, 2 and 53, respectively;
(26) SEQ ID NOs: 1, 52, 2 and 53, respectively;
(27) SEQ ID NOs: 1, 51, 2 and 54, respectively;
(28) SEQ ID NOs: 1, 52, 2 and 54, respectively;
(29) SEQ ID NOs: 1, 55, 2 and 57, respectively;
(30) SEQ ID NOs: 1, 56, 2 and 57, respectively;
(31) SEQ ID NOs: 1, 55, 2 and 58, respectively;
(32) SEQ ID NOs: 1, 56, 2 and 58, respectively;
(33) SEQ ID NOs: 23, 27, 24 and 29, respectively;
(34) SEQ ID NOs: 23, 28, 24 and 29, respectively;
(35) SEQ ID NOs: 23, 27, 24 and 30, respectively;
(36) SEQ ID NOs: 23, 28, 24 and 30, respectively;
(37) SEQ ID NOs: 25, 31, 26 and 33, respectively;
(38) SEQ ID NOs: 25, 32, 26 and 33, respectively;
(39) SEQ ID NOs: 25, 31, 26 and 34, respectively;
(40) SEQ ID NOs: 25, 32, 26 and 34, respectively;
(41) SEQ ID NOs: 23, 35, 24 and 37, respectively;
(42) SEQ ID NOs: 23, 36, 24 and 37, respectively;
(43) SEQ ID NOs: 23, 35, 24 and 38, respectively;
(44) SEQ ID NOs: 23, 36, 24 and 38, respectively;
(45) SEQ ID NOs: 25, 39, 26 and 41, respectively;
(46) SEQ ID NOs: 25, 40, 26 and 41, respectively;
(47) SEQ ID NOs: 25, 39, 26 and 42, respectively;
(48) SEQ ID NOs: 25, 40, 26 and 42, respectively;
(49) SEQ ID NOs: 23, 43, 24 and 45, respectively;
(50) SEQ ID NOs: 23, 44, 24 and 45, respectively;
(51) SEQ ID NOs: 23, 43, 24 and 46 respectively;
(52) SEQ ID NOs: 23, 44, 24 and 46, respectively;
(53) SEQ ID NOs: 25, 47, 26 and 49, respectively;
(54) SEQ ID NOs: 25, 48, 26 and 49, respectively;
(55) SEQ ID NOs: 25, 47, 26 and 50, respectively;
(56) SEQ ID NOs: 25, 48, 26 and 50, respectively;
(57) SEQ ID NOs: 23, 51, 24 and 53, respectively;
(58) SEQ ID NOs: 23, 52, 24 and 53, respectively;
(59) SEQ ID NOs: 23, 51, 24 and 54, respectively;
(60) SEQ ID NOs: 23, 52, 24 and 54, respectively;
(61) SEQ ID NOs: 25, 55, 26 and 57, respectively;
(62) SEQ ID NOs: 25, 56, 26 and 57, respectively;
(63) SEQ ID NOs: 25, 55, 26 and 58, respectively; or (64) SEQ ID NOs: 25, 56, 26 and 58, respectively.

9. The isolated bispecific antibody or antigen-binding fragment thereof of claim 8, wherein the first antigen-binding domain comprises the VH, CH1, VL, and CL, the second antigen-binding domain comprises the VH, CH1, VL, and CL, comprising the amino acid sequences of:
(1) SEQ ID: 1, 28, 2, 29, 3, 63, 4 and 64, respectively;
(2) SEQ ID: 1, 36, 2, 37, 3, 67, 4 and 68, respectively;
(3) SEQ ID: 1, 44, 2, 45, 3, 71, 4 and 72, respectively;
(4) SEQ ID: 1, 52, 2, 53, 3, 73, 4 and 74, respectively;
(5) SEQ ID: 1, 31, 2, 34, 3, 65, 4 and 66, respectively;
(6) SEQ ID: 1, 39, 2, 42, 3, 69, 4 and 70, respectively;
(7) SEQ ID: 1, 47, 2, 50, 3, 75, 4 and 76, respectively;
(8) SEQ ID: 1, 55, 2, 58, 3, 77, 4 and 78, respectively;
(9) SEQ ID: 23, 28, 24, 29, 59, 63, 60 and 64, respectively;
(10) SEQ ID: 23, 36, 24, 37, 59, 67, 60 and 68, respectively;
(11) SEQ ID: 23, 44, 24, 45, 59, 71, 60 and 72, respectively;
(12) SEQ ID: 23, 52, 24, 53, 59, 73, 60 and 74, respectively;
(13) SEQ ID: 25, 31, 26, 34, 61, 65, 62 and 66, respectively;
(14) SEQ ID: 25, 39, 26, 42, 61, 69, 62 and 70, respectively;
(15) SEQ ID: 25, 47, 26, 50, 61, 75, 62 and 76, respectively; or (16) SEQ ID: 25, 55, 26, 58, 61, 77, 62 and 78, respectively.

10. The isolated bispecific antibody or antigen-binding fragment thereof of claim 1, wherein the isolated bispecific antibody or antigen-binding fragment thereof comprises an anti-immune cell modulator (ICM) antibody or antigen-binding fragment arm thereof and is capable of specific binding to the ICM.

11. The isolated bispecific antibody or antigen-binding fragment thereof of claim 10, wherein the ICM is selected from the group consisting of CD3, CD16, CD27, CD28, CD40, CD122, NKp46, OX40, 4-1BB, GITR, ICOS, CTLA-4, PD-1, LAG-3, TIM-3, VISTA, SIGLEC7, SIGLEC9, KIR, BTLA, B7-H3, and other cell surface immune regulatory molecules.

12. The isolated bispecific antibody or antigen-binding fragment thereof of claim 1, wherein the first antigen-binding domain specifically binds CD3, and comprises the VH, CH1, VL, and CL comprising the amino acid sequences of SEQ ID: 9, 10, 11 and 12, respectively.

13. The isolated bispecific antibody or antigen-binding fragment thereof of claim 1, wherein the first antigen-binding domain specifically binds CD3, and the second antigen-binding domain specifically binds DLL3.

14. The isolated bispecific antibody or antigen-binding fragment thereof of claim 13, wherein the first antigen-binding domain comprises the VH, CH1, VL, and CL comprising the amino acid sequences of SEQ ID:9, 10, 11 and 12, respectively, and the second antigen-binding domain comprises the VH, CH1, VL, and CL comprising the amino acid sequences of SEQ ID:13, 14, 15 and 16, respectively.

15. An isolated nucleic acid encoding the isolated bispecific antibodies or antigen-binding fragments thereof of claim 1.

16. A vector comprising the isolated nucleic acid thereof of claim 15.

17. A host cell comprising the vector thereof of claim 16.

18. A pharmaceutical composition, comprising the isolated bispecific antibody or antigen-binding fragment thereof of claim 1 and a pharmaceutically acceptable carrier.

19. A method of targeting DLL3 that is expressed on a cancer cell surface in a subject in need thereof by engaging T cells, comprising administering to the subject a pharmaceutical composition thereof of claim 18.

20. A method of targeting one or two antigens expressed on a cancer cell surface in a subject in need thereof including using CD47 blockade induced activation of macrophage-mediated phagocytosis or CD3-mediated T cell activation, and/or treating cancer in a subject in need thereof, comprising administering to the subject a pharmaceutical composition comprising the isolated bispecific antibody or antigen-binding fragment thereof of claim 1 and a pharmaceutically acceptable carrier, optionally the cancer is selected from the group consisting of a lung cancer, a gastric cancer, an esophageal cancer, a bile duct cancer, a cholangiocarcinoma, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CIVIL), a multiple myeloma (MM), an acute myeloid leukemia (AML), and other liquid tumors.

21. A method of producing the bispecific antibody or antigen-binding fragment thereof of claim 1, comprising culturing a cell comprising a nucleic acid encoding the bispecific antibody or antigen-binding fragment thereof under conditions to produce the bispecific antibody or antigen-binding fragment thereof, and recovering the bispecific antibody or antigen-binding fragment thereof from the cell or culture.

22. A method of producing a pharmaceutical composition comprising the bispecific antibody or antigen-binding fragment thereof of claim 1, comprising combining the bispecific antibody or antigen-binding fragment thereof with a pharmaceutically acceptable carrier to obtain the pharmaceutical composition.
